(19) United States
(12) Patent Application Publication
(10) Pub. No.: US 2026/0069538 A1
(43) Pub. Date: Mar. 12, 2026

(54) CXCR4 ANTAGONIST LOADED LIPOSOMES AND SILICASOMES

(71) Applicant: The Regents of the University of California, Oakland, CA (US)

(72) Inventors: Andre E. NEL, Sherman Oaks, CA (US); Kuo-Ching MEI, Los Angeles, CA (US); Xiangsheng LIU, Los Angeles, CA (US); Yu-Pei LIAO, Culver City, CA (US)

(73) Assignee: The Regents of the University of California, Oakland, CA (US)

(21) Appl. No.: 18/707,333

(22) PCT Filed: Nov. 8, 2022
(86) PCT No.: PCT/US2022/049281

§ 371 Date: May 3, 2024

Related U.S. Application Data
(60) Provisional application No. 63/277,112, filed on Nov. 8, 2021.

(51) Int. Cl. A61K 9/127; A61K 31/122; A61K 31/136; A61K 31/155; A61K 31/282; A61K 31/337; A61K 31/352; A61K 31/395; A61K 31/4045; A61K 31/405; A61K 31/41; A61K 31/4178; A61K 31/4188; A61K 31/4245; A61K 31/437; A61K 31/4427; A61K 31/444; A61K 31/4709; A61K 31/675; A61K 31/69; A61K 31/704; A61K 33/243; A61K 38/14; A61K 47/54; A61K 47/69; A61P 35/00

(52) U.S. Cl. CPC A61K 9/127; A61K 31/122; A61K 31/136; A61K 31/155; A61K 31/282; A61K 31/337; A61K 31/352; A61K 31/395; A61K 31/4045; A61K 31/405; A61K 31/41; A61K 31/4178; A61K 31/4188; A61K 31/4245; A61K 31/437; A61K 31/4427; A61K 31/444; A61K 31/4709; A61K 31/675; A61K 31/69; A61K 31/704; A61K 33/243; A61K 38/14; A61K 47/545; A61K 47/6911; A61P 35/00

(57) ABSTRACT

In various embodiments drug delivery vehicles and uses thereof are provided. In certain embodiments the drug delivery vehicles comprise: 1) a silicasome comprising a mesoporous silica nanoparticle coated with a lipid bilayer and further comprising a CXCR4 antagonist; or 2) a liposome comprising a lipid bilayer comprising where said liposome further comprises a CXCR4 antagonist. In certain embodiments the CXCR4 antagonists are selected from the group consisting of AMD3100, AMD3465, and AMD070.

### CROSS-REFERENCE TO RELATED APPLICATIONS

This application claims priority to and benefit of U.S. Ser. No. 63/277,112, filed on Nov. 8, 2021, which is incorporated herein by reference in its entirety for all purposes.

### STATEMENT OF GOVERNMENTAL SUPPORT

[Not Applicable]

### BACKGROUND

A great deal of effort is currently directed to characterizing the tumor microenvironment (TME) of solid cancers, including identification of heterogeneous immune landscapes to initiate custom-designed immunotherapy (FIG. 1) (see, e.g., Allott et al. (2021) Cancers (Basel) 13(22): 5752; Picard et al. (2020) Front. Immunol. 11: 369). While the introduction of immune checkpoint inhibitors has advanced immunotherapy as one of the cornerstones of cancer treatment, we have come to understand that its success depends on the immunogenic nature of the tumor as well as the makeup of the tumor immune microenvironment (TIME) (see, e.g., Picard et al. (2020) Front. Immunol. 11: 369; Yang & Zhang (2020) Wiley Interdiscip. Rev. Nanomed. Nanobiotechnol. 12: e1612; Rodallec et al. (2020) Front. Immunol. 11: 784). Generally speaking, immune inflamed or “hot” tumors are associated with better responses to immune checkpoint inhibitors (ICIs) in cancers such as melanoma, non-small cell lung cancer, head and neck cancer, kidney, liver, and bladder cancer (FIG. 1) (see, e.g., Rodallec et al. (2020) Front. Immunol. 11: 784; Sevenich et al. (2019) Front. Oncol. 9: 163; Michel et al. (2020) Target Oncol. 15: 415-428). In contrast, immunological “cold” tumors exhibit a paucity of T-cell infiltrates (also referred to as “immune desert” landscapes) or present a phenotype where T-cells may be present but excluded from the tumor core, a.k.a. “immune excluded” landscapes (FIG. 1) (see, e.g., Gruosso et al. (2019) J. Clin. Invest. 129: 1785-1800; Hegde et al. (2016) Clin. Cancer Res. 22: 1865-1874). In addition to spatial exclusion, there are a number of additional reasons why the function of tumor-infiltrating T lymphocytes (TIL) at the tumor site may be constrained from cytotoxic killing, e.g., as a result of the expression of immune checkpoint receptors or immune metabolic interference by the indoleamine-pyrrole 2,3-dioxygenase or IDO-1) pathway. It is also important to consider the role of the dysplastic tumor stroma in exerting immune suppressive effects in the TME through the participation of cancer-associated fibroblasts (CAFs), myeloid derived suppressor cells (MDSC), FoxP3 regulatory T-cells and M2 tumor-associated macrophages (TAM).

Against this background, we asked whether tumor heterogeneity can be therapeutically targeted or exploited to improve combination therapy in the era of immune checkpoint blockers? Not only does this require knowledge of the makeup of heterogeneous tumor landscapes, but also provides a rational approach for combining active pharmaceutical ingredients (API) to reprogram “cold” immune landscapes, overcome T-cell exclusion, overcome T-cell exhaustion by checkpoint receptors, circumvent IDO-1 suppression, and address the immune suppressive properties of the tumor stroma. While currently these challenges are being addressed by theory, immune phenotyping and modeling, it is a challenge to replicate the complexity of the TIME outside the human body, leading to the conceptualization of therapeutic combinations beyond the number of study subjects that are available to conduct these studies.

### SUMMARY

Various embodiments provided herein may include, but need not be limited to, one or more of the following:

Embodiment 1: A drug delivery vehicle, said drug delivery vehicle comprising:
a silicasome comprising a mesoporous silica nanoparticle coated with a lipid bilayer and further comprising a CXCR4 antagonist; or
a liposome comprising a lipid bilayer comprising where said liposome further comprises a CXCR4 antagonist.

Embodiment 2: The drug delivery vehicle of embodiment 1, wherein said drug delivery vehicle comprises a silicasome comprising a mesoporous silica nanoparticle coated with a lipid bilayer and further comprising a CXCR4 antagonist.

Embodiment 3: The drug delivery vehicle of embodiment 1, wherein said drug delivery vehicle comprises a liposome comprising a lipid bilayer comprising where said liposome further comprises a CXCR4 antagonist.

Embodiment 4: The drug delivery vehicle according to any one of embodiments 1-3, wherein said CXCR4 antagonist comprises one or more CXCR4 antagonists selected from the group consisting of AMD3100, AMD3465, and AMD070.

Embodiment 5: The drug delivery vehicle of embodiment 4, wherein said CXCR4 antagonist comprises AMD3100.

Embodiment 6: The drug delivery vehicle of embodiment 4, wherein said CXCR4 antagonist comprises AMD3465.

Embodiment 7: The drug delivery vehicle of embodiment 4, wherein said CXCR4 antagonist comprises AMD070.

Embodiment 8: The drug delivery vehicle according to any one of embodiments 1-7, wherein said CXCR4 antagonist is disposed within said silicasome or said liposome.

Embodiment 9: The drug delivery vehicle of embodiment 8, wherein the CXCR4 antagonist is remote loaded into said silicasome or said liposome using a protonating agent.

Embodiment 10: The drug delivery vehicle of embodiment 9, wherein said protonating agent before reaction with the CXCR4 antagonist is selected from the group consisting of triethylammonium sucrose octasulfate (TEA₈SOS), (NH₄)₂SO₄, an ammonium salt, a trimethylammonium salt, and a triethylammonium salt.

Embodiment 11: The drug delivery vehicle of embodiment 10, wherein said protonating agent is ammonium sulfate.

Embodiment 12: The drug delivery vehicle of embodiment 10, wherein said protonating agent is triethylammonium sucrose octasulfate (TEA₈SOS).

Embodiment 13: The drug delivery vehicle according to any one of embodiments 1-12, wherein said CXCR4 antagonist is conjugated to a component of the lipid bilayer comprising said silicasome or said liposome.

Embodiment 14: The drug delivery vehicle of embodiment 13, wherein said CXCR4 antagonist is conjugated to a component of the lipid bilayer selected from the group consisting of a phospholipid, cholesterol, a cholesterol derivative, and a pegylated lipid.

Embodiment 15: The drug delivery vehicle according to any one of embodiments 1-14, wherein said lipid bilayer comprises a phospholipid and/or a phospholipid prodrug.

Embodiment 16: The drug delivery vehicle of embodiment 15, wherein said lipid bilayer comprises a phospholipid, and cholesterol (CHOL) and/or a cholesterol derivative.

Embodiment 17: The drug delivery vehicle according to any one of embodiments 15-16, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

Embodiment 18: The drug delivery vehicle of embodiment 17, wherein said phospholipid comprises a saturated fatty acid selected from the group consisting of phosphatidylcholine (DPPC), dimyristoylphosphatidylcholine (DMPC), distearoylphosphatidylcholine (DSPC), diactylphosphatidylcholine (DAPC), and 1,2-distearoyl-sn-glycero-3-phosphoethanolamine (DSPE).

Embodiment 19: The drug delivery vehicle of embodiment 17, wherein said phospholipid comprises a natural lipid selected from the group consisting of egg phosphatidylcholine (egg PC), and soy phosphatidylcholine (soy PC).

Embodiment 20: The drug delivery vehicle of embodiment 17, wherein said phospholipid comprises an unsaturated fatty acid selected from the group consisting of 1,2-dimyristoleoyl-sn-glycero-3-phosphocholine, 1,2-dipalmitoleoyl-sn-glycero-3-phosphocholine, 1,2-dioleoyl-sn-glycero-3-phosphocholine (DOPC), and 1,2-dieicosenoyl-sn-glycero-3-phosphocholine.

Embodiment 21: The drug delivery vehicle according to any one of embodiments 15-20, wherein said lipid bilayer comprises an mPEG phospholipid with a phospholipid C14-C18 carbon chain, and a PEG molecular weight ranging from about 350 Da to 5000 Da.

Embodiment 22: The drug delivery vehicle of embodiment 21, wherein said lipid bilayer comprises 1,2-distearoyl-sn-glycero-3-phosphoethanolamine-PEG (DSPE-PEG).

Embodiment 23: The drug delivery vehicle of embodiment 22, wherein said lipid bilayer comprises DPSE-PEG_{2K}.

Embodiment 24: The drug delivery vehicle according to any one of embodiments 15-23, wherein said lipid bilayer comprises a cholesterol derivative.

Embodiment 25: The drug delivery vehicle of embodiment 24, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

Embodiment 26: The drug delivery vehicle of embodiment 25, wherein said lipid bilayer comprises cholesterol hemisuccinate (CHEMS).

Embodiment 27: The drug delivery vehicle of embodiment 18, wherein said lipid bilayer comprises DSPC and cholesterol (Chol).

Embodiment 28: The drug delivery vehicle of embodiment 27, wherein said lipid bilayer comprises DSPC, cholesterol (Chol), and a pegylated lipid.

Embodiment 29: The drug delivery vehicle of embodiment 28, wherein the molar ratio of DSPC:Chol:Pegylated lipid is 3:2:0.15.

Embodiment 30: The drug delivery vehicle according to any one of embodiments 28-29, wherein said lipid bilayer comprises DSPC, Chol, and DSPE-PEG.

Embodiment 31: The drug delivery vehicle of embodiment 30, wherein said lipid bilayer comprises DSPC, Chol, and DSPE-PEG2000.

Embodiment 32: The drug delivery vehicle according to any one of embodiments 1-31, wherein said drug delivery vehicle is conjugated to a moiety selected from the group consisting of a targeting moiety, a fusogenic peptide, and a transport peptide.

Embodiment 33: The drug delivery vehicle of embodiment 32, wherein said drug delivery vehicle is conjugated to a peptide that binds a receptor on a cancer cell or tumor blood vessel.

Embodiment 34: The drug delivery vehicle of embodiment 33, wherein said drug delivery vehicle is conjugated to an iRGD peptide.

Embodiment 35: The drug delivery vehicle of embodiment 33, wherein said drug delivery vehicle is conjugated to a targeting peptide shown in Table 4.

Embodiment 36: The drug delivery vehicle according to any one of embodiments 32-35, wherein said drug carrier is conjugated to transferrin, and/or ApoE, and/or folate.

Embodiment 37: The drug delivery vehicle according to any one of embodiments 32-36, wherein said drug carrier is conjugated to a targeting moiety that comprises an antibody that binds to a cancer marker.

Embodiment 38: The drug delivery vehicle of embodiment 37, wherein said drug carrier is conjugated to a targeting moiety that comprises an antibody that binds a cancer marker shown in Table 3.

Embodiment 39: The drug delivery vehicle according to any one of embodiments 37-38, wherein said antibody is selected from the group consisting of an intact immunoglobulin, an F(ab)′₂, a Fab, a single chain antibody, a diabody, an affibody, a unibody, and a nanobody.

Embodiment 40: The drug delivery vehicle according to any one of embodiments 1-39, wherein said drug carriers in suspension are stable for at least 1 month, or at least 2 months, or at least 3 months, or at least 4 months, or at least 5 months, or at least 6 months when stored at 4° C.

Embodiment 41: The drug delivery vehicle according to any one of embodiments 1-40, wherein said nanoparticle drug carrier forms a stable suspension on rehydration after lyophilization.

Embodiment 42: The drug delivery vehicle according to any one of embodiments 1-41, wherein said nanoparticle drug carriers, show reduced drug toxicity as compared to free drug.

Embodiment 43: The drug delivery vehicle according to any one of embodiments 1-42, wherein said nanoparticle drug carrier has colloidal stability in physiological fluids with pH 7.4 and remains monodisperse to allow systemic biodistribution and is capable of entering a disease site by vascular leakage (EPR effect) or transcytosis.

Embodiment 44: The drug delivery vehicle according to any one of embodiments 1-43, wherein said drug delivery vehicle contains a second drug or said second drug is conjugated to a component of the lipid bilayer comprising said silicasome or said liposome.

Embodiment 45: The drug delivery vehicle of embodiment 44, wherein said drug delivery vehicle contains said second drug.

Embodiment 46: The drug delivery vehicle of embodiment 44, wherein said second drug is conjugated to a component of the lipid bilayer comprising said silicasome or said liposome.

Embodiment 47: The drug delivery vehicle of embodiment 46, wherein said second drug is conjugated to a component of the lipid bilayer selected from the group consisting of a phospholipid, cholesterol, a cholesterol derivative, and a pegylated lipid.

Embodiment 48: The drug delivery vehicle of embodiment 47, wherein second drug is conjugated directly to said component of the lipid bilayer.

Embodiment 49: The drug delivery vehicle of embodiment 47, wherein second drug is conjugated to said component of the lipid bilayer via a linker.

Embodiment 50: The drug delivery vehicle according to any one of embodiments 47-49, wherein said second drug is conjugated to a phospholipid.

Embodiment 51: The drug delivery vehicle according to any one of embodiments 47-49, wherein said second drug is conjugated to cholesterol.

Embodiment 52: The drug delivery vehicle according to any one of embodiments 47-49, wherein said second drug is conjugated to a cholesterol derivative.

Embodiment 53: The drug delivery vehicle according to any one of embodiments 47-49, wherein said second drug is conjugated to a pegylated lipid.

Embodiment 54: The drug delivery vehicle of embodiment 53, wherein said second drug is conjugated to DSPE-PEG.

Embodiment 55: The drug delivery vehicle according to any one of embodiments 44-54, wherein said second drug comprises one or more drugs selected from the group consisting of an IDO inhibitor, an immunogenic cell death (ICD)-inducing drug.

Embodiment 56: The drug delivery vehicle of embodiment 55, wherein said second drug comprises one or more IDO inhibitor(s).

Embodiment 57: The drug delivery vehicle of embodiment 56, wherein said IDO-1 inhibitor comprises an agent selected from the group consisting of D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-β-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide, N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, and 4-phenylimidazole.

Embodiment 58: The drug delivery vehicle of embodiment 57, wherein said IDO inhibitor comprises 1-methyl-tryptophan.

Embodiment 59: The drug delivery vehicle of embodiment 58, wherein said IDO inhibitor comprises a “D” enantiomer of 1-methyl-tryptophan (indoximod, 1-MT).

Embodiment 60: The drug delivery vehicle of embodiment 58, wherein said IDO inhibitor comprises an “L” enantiomer of 1-methyl-tryptophan (L-MT).

Embodiment 61: The drug delivery vehicle according to any one of embodiments 55-60, wherein said second drug comprises one or more ICD inducer(s).

Embodiment 62: The drug delivery vehicle of embodiment 61, wherein said ICD inducer comprises a chemotherapeutic agent selected from the group consisting of doxorubicin, oxaliplatin, anthracenedione, bleomycin, bortezomib, cisplatin, daunorubicin, docetaxel, epirubicin, idarubicin, mitoxanthrone, paclitaxel, R2016, and cyclophosphamide.

Embodiment 63: The drug delivery vehicle of embodiment 62, wherein said ICD inducer comprises doxorubicin.

Embodiment 64: The drug delivery vehicle of embodiment 62, wherein said ICD inducer comprises oxaliplatin.

Embodiment 65: The drug delivery vehicle of embodiment 62, wherein said ICD inducer comprises mitoxanthrone.

Embodiment 66: The drug delivery vehicle according to any one of embodiments 55-65, wherein said second drug comprises an immune checkpoint inhibitor (ICI).

Embodiment 67: The drug delivery vehicle of embodiment 66, wherein said checkpoint inhibitor comprises one or more checkpoint inhibitors selected from the group consisting of a PD-L1 inhibitor, a PD-1 inhibitor, a CTLA-4 inhibitor, a PD-L2inhibitor, a PD-L3inhibitor, a PD-L4inhibitor, a LAG3inhibitor, a B7-H3inhibitor, a B7-H4inhibitor, a KIR, and a TIM3 inhibitor.

Embodiment 68: The drug delivery vehicle of embodiment 67, wherein said checkpoint inhibitor comprises one or more PD-L1 inhibitors.

Embodiment 69: The drug delivery vehicle of embodiment 68, wherein said checkpoint inhibitor comprises an anti-PD-L1 antibody.

Embodiment 70: The drug delivery vehicle of embodiment 69, wherein said checkpoint inhibitor comprises an anti-PD-L1 antibody selected from the group consisting of Atezolizumab, Avelumab, Durvalumab, BMS-936559, RG-7446. MPDL3280A, MEDI-4736, and MSB0010718C.

Embodiment 71: The drug delivery vehicle of embodiment 68, wherein said checkpoint inhibitor comprises a peptidic PD-L1 inhibitor.

Embodiment 72: The drug delivery vehicle of embodiment 71, wherein said PD-L1 inhibitor comprise a moiety selected from the group consisting of AUNP12, CA-170, and BMS-986189.

Embodiment 73: The drug delivery vehicle according to any one of embodiments 67-72, wherein said checkpoint inhibitor comprises a PD1 inhibitor.

Embodiment 74: The drug delivery vehicle of embodiment 73, wherein said checkpoint inhibitor comprises an anti-PD1 antibody.

Embodiment 75: The drug delivery vehicle of embodiment 74, wherein said checkpoint inhibitor comprises an anti-PD1 antibody selected from the group consisting of Nivolumab, Pembrolizumab, Cemiplimab, avelumab, durvalumab, and atezolizumab.

Embodiment 76: The drug delivery vehicle of embodiment 73, wherein said checkpoint inhibitor comprises an Fc fusion with PD-L2.

Embodiment 77: The drug delivery vehicle of embodiment 76, wherein said checkpoint inhibitor comprises AMP224.

Embodiment 78: The drug delivery vehicle according to any one of embodiments 67-77, wherein said checkpoint inhibitor comprises CTLA-4 inhibitor.

Embodiment 79: The drug delivery vehicle of embodiment 78, wherein said CTLA-4 inhibitor comprises Ipilimumab.

Embodiment 80: The drug delivery vehicle according to any one of embodiments 55-79, wherein said second drug comprises a GSK3 inhibitor.

Embodiment 81: The drug delivery vehicle of embodiment 80, wherein said GSK3 inhibitor comprises a weak basic GS3K inhibitor.

Embodiment 82: The drug delivery vehicle of embodiment 81, wherein said GS3K inhibitor is selected from the group consisting of AZD2858, AZD1080, LY2090314, and 1-Azakenpaullone.

Embodiment 83: The drug delivery vehicle of embodiment 82, wherein said GS3K inhibitor comprises AZD1080.

Embodiment 84: A pharmaceutical formulation, said formulation comprising:
a drug delivery vehicle according to any one of embodiments 1-83; and
a pharmaceutically acceptable carrier.

Embodiment 85: The pharmaceutical formulation of embodiment 84, wherein said formulation is an emulsion, dispersion, or suspension.

Embodiment 86: The pharmaceutical formulation of embodiment 85, wherein said suspension, emulsion, or dispersion is stable for at least 1 month, or at least 2 months, or at least 3 months, or at least 4 months, or at least 5 months, or at least 6 months when stored at 4° C.

Embodiment 87: The pharmaceutical formulation according to any one of embodiments 84-86, wherein the drug delivery vehicle in said formulation shows a substantially unimodal size distribution; and/or show a PDI less than about 0.2, or less than about 0.1.

Embodiment 88: The pharmaceutical formulation according to any one of embodiments 84-87, wherein said formulation is formulated for administration via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

Embodiment 89: The pharmaceutical formulation according to any one of embodiments 84-87, wherein said formulation is a sterile injectable.

Embodiment 90: The pharmaceutical formulation according to any one of embodiments 84-89, wherein said formulation is a unit dosage formulation.

Embodiment 91: A method of treating a cancer, said method comprising:
administering to a subject in need thereof an effective amount of a drug delivery vehicle according to any one of embodiments 1-83 or a pharmaceutical formulation according to any one of embodiments 84-90.

Embodiment 92: The method of embodiment 91, wherein said administering to a subject in need thereof an effective amount of a drug delivery vehicle comprises a primary therapy in a chemotherapeutic regimen.

Embodiment 93: The method of embodiment 91, wherein said administering to a subject in need thereof an effective amount of a drug delivery vehicle comprises an adjunct therapy in a treatment regime that additionally comprises chemotherapy using another chemotherapeutic agent, and/or surgical resection of a tumor mass, and/or radiotherapy.

Embodiment 94: The method of embodiment 91, wherein said drug delivery vehicle and/or said pharmaceutical formulation is a component in a multi-drug chemotherapeutic regimen.

Embodiment 95: The method according to any one of embodiments 91-94, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

Embodiment 96: The method of embodiment 95, wherein said cancer is breast cancer.

Embodiment 97: The method of embodiment 95, wherein said cancer is triple negative breast cancer.

Embodiment 98: The method of embodiment 95, wherein said cancer is pancreatic ductal adenocarcinoma (PDAC).

Embodiment 99: The method according to any one of embodiments 91-98, wherein said administration is via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition.

Embodiment 100: The method according to any one of embodiments 91-98, wherein said administration comprises systemic administration via injection or cannula.

Embodiment 101: The method according to any one of embodiments 91-98, wherein said administration is administration to an intra-tumoral or peri-tumoral site.

Embodiment 102: The method according to any one of embodiments 91-101, wherein said mammal is a human.

Embodiment 103: The method according to any one of embodiments 91-101, wherein said mammal is a non-human mammal.

Embodiment 104: The method according to any one of embodiments 91-103, wherein said drug delivery vehicle is coadminstered with a second drug.

Embodiment 105: The method of embodiment 104, wherein said coadministration is simultaneous coadminstration.

Embodiment 106: The method of embodiment 104, wherein said drug delivery vehicle and said second drug are administered at different times.

Embodiment 107: The method according to any one of embodiments 104-106, wherein said second drug comprises one or more drugs selected from the group consisting of an IDO-1 inhibitor, an immunogenic cell death (ICD)-inducing drug.

Embodiment 108: The method of embodiment 107, wherein said second drug comprises one or more IDO inhibitor(s).

Embodiment 109: The method of embodiment 108, wherein said IDO-1 inhibitor comprises an agent selected from the group consisting of D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-β-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide, N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, and 4-phenylimidazole.

Embodiment 110: The method of embodiment 109, wherein said IDO inhibitor comprises 1-methyl-tryptophan.

Embodiment 111: The method of embodiment 110, wherein said IDO inhibitor comprises a “D” enantiomer of 1-methyl-tryptophan (indoximod, 1-MT).

Embodiment 112: The method of embodiment 110, wherein said IDO inhibitor comprises an “L” enantiomer of 1-methyl-tryptophan (L-MT).

Embodiment 113: The method according to any one of embodiments 107-112, wherein said second drug comprises one or more ICD inducer(s).

Embodiment 114: The method of embodiment 113, wherein said ICD inducer comprises a chemotherapeutic agent selected from the group consisting of doxorubicin, oxaliplatin, anthracenedione, bleomycin, bortezomib, cisplatin, daunorubicin, docetaxel, epirubicin, idarubicin, mitoxanthrone, paclitaxel, R2016, and cyclophosphamide.

Embodiment 115: The method of embodiment 114, wherein said ICD inducer comprises doxorubicin.

Embodiment 116: The method of embodiment 114, wherein said ICD inducer comprises oxaliplatin.

Embodiment 117: The method of embodiment 114, wherein said ICD inducer comprises mitoxanthone.

Embodiment 118: The method according to any one of embodiments 107-117, wherein said second drug comprises an immune checkpoint inhibitor (ICI).

Embodiment 119: The method of embodiment 118, wherein said checkpoint inhibitor comprises one or more checkpoint inhibitors selected from the group consisting of a PD-L1 inhibitor, a PD-1 inhibitor, a CTLA-4 inhibitor, a PD-L2inhibitor, a PD-L3inhibitor, a PD-L4inhibitor, a LAG3inhibitor, a B7-H3inhibitor, a B7-H4inhibitor, a KIR, and a TIM3 inhibitor.

Embodiment 120: The method of embodiment 119, wherein said checkpoint inhibitor comprises one or more PD-L1 inhibitors.

Embodiment 121: The method of embodiment 120, wherein said checkpoint inhibitor comprises an anti-PD-L1 antibody.

Embodiment 122: The method of embodiment 121, wherein said checkpoint inhibitor comprises an anti-PD-L1 antibody selected from the group consisting of Atezolizumab, Avelumab, Durvalumab, BMS-936559, RG-7446. MPDL3280A, MEDI-4736, and MSB0010718C.

Embodiment 123: The method of embodiment 120, wherein said checkpoint inhibitor comprises a peptidic PD-L1 inhibitor.

Embodiment 124: The method of embodiment 123, wherein said PD-L1 inhibitor comprise a moiety selected from the group consisting of AUNP12, CA-170, and BMS-986189.

Embodiment 125: The method according to any one of embodiments 119-124, wherein said checkpoint inhibitor comprises a PD1 inhibitor.

Embodiment 126: The method of embodiment 125, wherein said checkpoint inhibitor comprises an anti-PD1 antibody.

Embodiment 127: The method of embodiment 126, wherein said checkpoint inhibitor comprises an anti-PD1 antibody selected from the group consisting of Nivolumab, Pembrolizumab, Cemiplimab, avelumab, durvalumab, and atezolizumab.

Embodiment 128: The method of embodiment 125, wherein said checkpoint inhibitor comprises an fc fusion with PD-L2.

Embodiment 129: The method of embodiment 128, wherein said checkpoint inhibitor comprises AMP224.

Embodiment 130: The method according to any one of embodiments 118-129, wherein said checkpoint inhibitor comprises CTLA-4 inhibitor.

Embodiment 131: The method of embodiment 130, wherein said CTLA-4 inhibitor comprises Ipilimumab.

Embodiment 132: The method according to any one of embodiments 107-131, wherein said second drug comprises a GSK3 inhibitor.

Embodiment 133: The method of embodiment 132, wherein said GSK3 inhibitor comprises a weak basic GS3K inhibitor.

Embodiment 134: The method of embodiment 133, wherein said GS3K inhibitor is selected from the group consisting of AZD2858, AZD1080, LY2090314, and 1-Azakenpaullone.

Embodiment 135: The method of embodiment 134, wherein said GS3K inhibitor comprises AZD1080.

### Definitions

The term “CXCR4 antagonist” refers to a substance that prevents activation of the CXCR4 receptor. In certain embodiments the CXCR4 antagonist blocks the CXCR4 receptor. Antagonizing (e.g., blocking) the receptor stops the receptor's ligand, CXCL12, from binding which prevents receptor signaling and downstream effects.

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that suffers from, or is at risk for a cancer as described herein. Thus, for example, in certain embodiments the subject is a subject with a cancer (e.g., pancreatic ductal adenocarcinoma (PDAC), breast cancer (e.g., drug-resistant breast cancer), colon cancer, brain cancer, and the like). In certain embodiments the methods described herein are prophylactic and the subject is one in whom a cancer is to be inhibited or prevented. In certain embodiments the subject for prophylaxis is one with a family history of cancer and/or a risk factor for a cancer (e.g., a genetic risk factor, an environmental exposure, and the like).

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term treat can refer to prophylactic treatment which includes a delay in the onset or the prevention of the onset of a pathology or disease.

The terms “coadministration” or “administration in conjunction with” or “cotreatment” when used in reference to the coadministration of a first compound (or component) (e.g., an immunogenic cell death (ICD) inducer) and a second compound (or component) (e.g., an indoleamine 2, 3 dioxygenase (IDO) inhibitor) indicates that the first compound (or component) and the second compound (or component) are administered so that there is at least some chronological overlap in the biological activity of first compound and the second compound in the organism to which they are administered. Coadministration can include simultaneous administration or sequential administration. In sequential administration there may even be some substantial delay (e.g., minutes or even hours) between administration of the first compound and the second compound as long as their biological activities overlap. In certain embodiments, the coadminstration is over a time frame that permits the first compound and second compound to produce an enhanced therapeutic or prophylactic effect on the organism. In certain embodiments the enhanced effect is a synergistic effect.

The term “immunogenic cell death” or “ICD” refers to a unique form of cell death caused by some cytostatic agents such as anthracyclines (Obeid et al. (2007) Nature Med., 13(1): 54-61), anthracenedione (mitoxantrone, aka MTX), oxaliplatin, irinotecan, and bortezomib, or radiotherapy and/or photodynamic therapy (PDT). Unlike regular apoptosis, which is mostly non-immunogenic or even tolerogenic, immunogenic apoptosis of cancer cells can induce an effective antitumor immune response through activation of dendritic cells (DCs) and consequent activation of specific T cell response (Spisek and Dhodapkar (2007) Cell Cycle, 6(16): 1962-1965). Endoplasmic reticulum (ER) stress, reactive oxygen species (ROS) production and induction of autophagy are key intracellular response pathways that govern ICD (Krysko et al. (2012) Nat. Rev. Canc. 12(12): 860-875). In addition to facilitating tumor cell death that facilitates antigen presentation by dendritic cells, ICD is characterized by secretion or release of damage-associated molecular patterns (DAMPs), which exert additional immune adjuvant effects. Calreticulin (CRT), one of the DAMP molecules, which is normally in the lumen of the ER, is translocated to the surface of dying cell where it functions as an “eat me” signal for phagocytes. Other important surface exposed DAMPs are heat-shock proteins (HSPs), namely HSP70 and HSP90, which under stress condition are also translocated to the plasma membrane. On the cell surface they have an immunostimulatory effect, based on their interaction with number of antigen-presenting cell (APC) surface receptors like CD91 and CD40 and also facilitate cross-presentation of antigens derived from tumor cells on MHC class I molecule, which then triggers CD8+ T cell activation and expansion. Other important DAMPs, characteristic for ICD are secreted amphoterin (HMGB1) and ATP (see, e.g., Apetoh et al. (2007) Nature Med. 13(9): 1050-1059; Ghiringhelli et al. (2009) Nature Med. 15(10): 1170-1178). HMGB1 is considered to be a late apoptotic marker and its release to the extracellular space appears to be required for the optimal release and presentation of tumor antigens to dendritic cells. It binds to several pattern recognition receptors (PRRs) such as Toll-like receptor (TLR) 2 and 4, which are expressed on APCs. The most recently found DAMP released during immunogenic cell death is ATP, which functions as a “find-me” signal for monocytes when secreted and induces their attraction to the site of apoptosis (see, e.g., Garg et al. (2012) EMBO J. 31(5): 1062-1079). ATP binds to purinergic receptors on APCs.

The terms “IDO inhibitor”, “IDO pathway inhibitor”, and “inhibitor of the IDO pathway) are used interchangeably and refer to an agent (a molecule or a composition) that either partially or fully blocks the activity of indoleamine-2,3-dioxygenase (IDO) and/or partially or fully suppresses the post-enzymatic signaling cascade(s) in the IDO pathway. IDO is an intracellular heme-containing enzyme that initiates the first and rate-limiting step of tryptophan degradation along the kynurenine pathway. The indoleamine 2,3-dioxygenase (IDO) pathway regulates immune response by suppressing cytotoxic T cell function, enhancing regulatory T cell activity (Tregs) and enabling tumor immune escape, either at the tumor or regional lymph node sites. An IDO pathway inhibitor can inhibit the IDO enzyme directly or by interfering or perturbing IDO effector pathway components. Such components include, but are not limited to: IDO2, tryptophan 2,3-dioxygenase (TDO), the mammalian target of rapamycin (mTOR) pathway, arylhydrocarbon receptor (AhR) pathway, the general control nonderepressible 2 (GCN2) pathway, and the AhR/IL-6 autocrine loop.

The terms “drug delivery vehicle”, “nanocarrier” and “nanoparticle drug carrier” are used interchangeably and refer nanostructures capable of delivering one or more drugs. Illustrative drug delivery vehicles include liposomes and silicasomes.

A “silicasome” refers to a particle having a porous interior core (e.g., a “porous nanoparticle”) that is encapsulated in a lipid bilayer. In certain embodiments the nanoparticle is a porous silica nanoparticle (e.g., mesoporous silica nanoparticle or “MSNP”). It will be recognized that in certain embodiments, the porous nanoparticle core can be fabricated from porous materials other than mesoporous silica.

As used herein, the term “lipid” refers to conventional lipids, phospholipids, cholesterol, chemically functionalized lipids for attachment of PEG and ligands, etc.

As used herein, the terms “lipid bilayer” or “LB” refers to any double layer of oriented amphipathic lipid molecules in which the hydrocarbon tails face inward to form a continuous non-polar phase.

As used herein, the terms “liposome” or “lipid vesicle” or “vesicle” are used interchangeably to refer to an aqueous compartment enclosed by a lipid bilayer, as being conventionally defined (see, e.g., Stryer (1981) Biochemistry, 2d Edition, W. H. Freeman & Co., p. 213).

Compared with the lipid bilayer coated on mesoporous silica nanoparticles (e.g., silicasomes), the lipid bilayer comprising a liposome can be referred to as an “unsupported lipid bilayer” and the lipid vesicle itself (when unloaded) can be referred to as an “empty vesicle”. The lipid bilayer coated on nanoparticles can be referred to as a “supported lipid bilayer” because the lipid bilayer is located on the surface and supported by a porous particle core. In certain embodiments, the lipid bilayer can have a thickness ranging from about 6 nm to about 7 nm which includes a 3-4 nm thickness of the hydrophobic core, plus the hydrated hydrophilic head group layers (each about 0.9 nm) plus two partially hydrated regions of about 0.3 nm each. In various embodiments, the lipid bilayer surrounding the silica nanoparticle comprises a continuous bilayer or substantially continuous bilayer that effectively encapsulates and seals the nanoparticle.

As used herein, the term “selective targeting” or “specific binding” refers to use of targeting ligands on the surface of a drug delivery nanocarrier (e.g., a LB-coated nanoparticle). In certain embodiments the targeting ligand(s) are on the surface of a lipid bilayer or LB-coated nanoparticle. Typically, the ligands interact specifically/selectively with receptors or other biomolecular components expressed on the target, e.g., a cell surface of interest. The targeting ligands can include such molecules and/or materials as peptides, antibodies, aptamers, targeting peptides, polysaccharides, and the like.

A coated mesoporous silica nanoparticle, having targeting ligands can be referred to as a “targeted nanoparticle or a targeted drug delivery nanocarrier (e.g., LB-coated nanoparticle).

The term “about” or “approximately” as used herein refers to being within an acceptable error range for the particular value as determined by one of ordinary skill in the art, which will depend in part on how the value is measured or determined, i.e. the limitations of the measurement system, i.e. the degree of precision required for a particular purpose, such as a pharmaceutical formulation. For example, “about” can mean within 1 or more than 1 standard deviation, per the practice in the art. Alternatively, “about” can mean a range of up to 20%, preferably up to 10%, more preferably up to 5% and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The term “drug” as used herein refers to a chemical entity of varying molecular size, small and large, naturally occurring or synthetic, that exhibits a therapeutic effect in animals and humans. A drug may include, but is not limited to, an organic molecule (e.g., a small organic molecule), a therapeutic protein, peptide, antigen, or other biomolecule, an oligonucleotide, an siRNA, a construct encoding CRISPR cas9 components and, optionally one or more guide RNAs, and the like.

A “pharmaceutically acceptable carrier” as used herein is defined as any of the standard pharmaceutically acceptable carriers. The pharmaceutical compositions of the subject invention can be formulated according to known methods for preparing pharmaceutically useful compositions. The pharmaceutically acceptable carrier can include diluents, adjuvants, and vehicles, as well as carriers, and inert, non-toxic solid or liquid fillers, diluents, or encapsulating material that does not react with the active ingredients of the invention. Examples include, but are not limited to: phosphate buffered saline, physiological saline, water, and emulsions, such as oil/water emulsions. The carrier can be a solvent or dispersing medium containing, for example, ethanol, polyol (for example, glycerol, propylene glycol, liquid polyethylene glycol, and the like), suitable mixtures thereof, and vegetable oils. Formulations are described in a number of sources that are well known and readily available to those skilled in the art. For example, Remington's Pharmaceutical Sciences (Martin E W [1995] Easton Pa., Mack Publishing Company, 19th ed.) describes formulations which can be used in connection with the drug delivery nanocarrier(s) (e.g., LB-coated nanoparticle(s)) described herein.

As used herein, an “antibody” refers to a protein consisting of one or more polypeptides substantially encoded by immunoglobulin genes or fragments of immunoglobulin genes or derived therefrom that is capable of binding (e.g., specifically binding) to a target (e.g., to a target polypeptide). The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Certain preferred antibodies include single chain antibodies (antibodies that exist as a single polypeptide chain), more preferably single chain Fv antibodies (sFv or scFv) in which a variable heavy and a variable light chain are joined together (directly or through a peptide linker) to form a continuous polypeptide. The single chain Fv antibody is a covalently linked V_{H}-V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. While the V_{H} and V_{L} are connected to each as a single polypeptide chain, the V_{H} and V_{L} domains associate non-covalently. The first functional antibody molecules to be expressed on the surface of filamentous phage were single-chain Fv's (scFv), however, alternative expression strategies have also been successful. For example Fab molecules can be displayed on a phage if one of the chains (heavy or light) is fused to g3 capsid protein and the complementary chain exported to the periplasm as a soluble molecule. The two chains can be encoded on the same or on different replicons; the important point is that the two antibody chains in each Fab molecule assemble post-translationally and the dimer is incorporated into the phage particle via linkage of one of the chains to, e.g., g3p (see, e.g., U.S. Pat. No. 5,733,743). The scFv antibodies and a number of other structures converting the naturally aggregated, but chemically separated light and heavy polypeptide chains from an antibody V region into a molecule that folds into a three-dimensional structure substantially similar to the structure of an antigen-binding site are known to those of skill in the art (see e.g., U.S. Pat. Nos. 5,091,513, 5,132,405, and 4,956,778). In certain embodiments antibodies should include all that have been displayed on phage (e.g., scFv, Fv, Fab and disulfide linked Fv (see, e.g, Reiter et al. (1995) Protein Eng. 8: 1323-1331) as well as affibodies, unibodies, and the like.

The term “specifically binds”, as used herein, when referring to a biomolecule (e.g., protein, nucleic acid, antibody, etc.), refers to a binding reaction that is determinative of the presence of a biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

### BRIEF DESCRIPTION OF THE DRAWINGS

FIG. 1 illustrates heterogeneous tumor immune microenvironments (TIMES). In in spite of the advances by immune checkpoint blocking antibodies for cancer immunotherapy, only 20-30% of patients with responsive cancers mount a robust antitumor immune response, provided that they exhibit an inflamed tumor microenvironment with CTL infiltration. To improve the response rate for these cancers and add to increase the overall number of additional cancers that can be successfully treated with checkpoint blocking antibodies, a number of approaches exist to convert “cold” tumors “hot”, including endogenous and exogenous vaccination approaches. Even when successful at improving CTL recruitment, these attempts may not be enough to achieve cytotoxic killing because of: (i) the immune suppressive effects of the tumor stroma; (ii) recruitment of CD8+ T-cells that are especially excluded from contacting PDAC or TNBC cancer cells; (iii) recruitment of CD8+ T-cells, which are put under constraint by ligation of checkpoint receptors on the immune metabolic effect of the IDO-1 pathway. Thus, in addition to inflamed (“hot”) and immune-depleted (“cold”, “immune desert” or “ignored”) TIMES at the far ends of the spectrum, intermediary categories such as “immune excluded”, “immune suppressed” and “immune escape” landscapes need to be considered for TNBC and PDAC immunotherapy. This requires customized design of treatment combinations to address the challenges in each landscape. Abbreviations: Treg=FoxP3+ regulatory T-cells; MDSC=myeloid derived suppressor cells; TAM=tumor-associated macrophages; IDO-1=Indoleamine-pyrrole 2,3-dioxygenase.

FIG. 2 shows DOX-NP effects on tumor growth in different TNBC mouse orthotopic models. BC cells were implanted into mammary pad of female Balb/c (4T1 and EMT6) and C57BL/6 (Py8119) mice (n=6). On day 8, 11 and 14, animals were IV injected with 5 mg/kg DOX-NP 3 times, commencing when the tumor size reached 100-150 mm³. Tumor growth was monitored by digital caliper.

FIG. 3 depicts DOX-NP effects on perforin in different TNBC mouse orthotopic models. BC cells were implanted into mammary pad of female Balb/c (4T1 and EMT6) and C57BL/6 (Py8119) mice (n=6). On day 8, 11 and 14, animals were IV injected with 5 mg/kg DOX-NP 3 times, commencing when the tumor size reached 100-150 mm³. Following animal sacrifice on day 16, tumors were harvested, and formalin-fixed for immunohistochemistry studies for perforin expression. Quantitative analysis of perforin positive stained cells was performed by Aperio ImageScope software.

FIG. 4 depicts DOX-NP effects on CRT in different TNBC mouse orthotopic models. BC cells were implanted into mammary pad of female Balb/c (4T1 and EMT6) and C57BL/6 (Py8119) mice (n=6). On day 8, 11 and 14, animals were IV injected with 5 mg/kg DOX-NP 3 times, commencing when the tumor size reached 100-150 mm³. Following animal sacrifice on day 16, tumors were harvested, and formalin-fixed for immunohistochemistry studies for CRT expression. Quantitative analysis of CRT positive stained cells was performed by Aperio ImageScope software.

FIG. 5, panels A-B, show CD8+ T-cell spatial distribution landscapes in 4T1, EMT6 and Py8119 TNBC animal models (Nel et al. (2022), ACS Nano. 16: 5184-5232). 4T1 (Balb/c), EMT6 (Balb/c) and Py8119 (C57BL/6) EC cells were orthotopically implanted in mouse mammary pads on day 0. When the tumors reached 100-150 mm3, animals were IV injected on days 8, 11 and 14 with DOX-NP (5 mg/kg; Avanti Polar Lipids) or left untreated (UT). Panel A: Tumors were collected on day 21 and analyzed by conventional IHC staining. Panel B) Quantitative analysis of CD8+ cells in tumor cores and margins during conventional IHC was performed, using Aperio ImageScope software. For mIHC analysis, tumor sections were stained with primary antibodies: CD8, α-SMA and Ki-67. Quantitative analysis of CD8+ numbers in cores and margins was performed using Akoya InForm Image Analysis software. Doxorubicin treatment induced increased CD8+ T-cell recruitment in all tumor types with both staining methods. Importantly, newly recruited CD8 T-cells tended to be margin- or stroma-restricted in EMT6 and Py8119 tumors, while CTL distribution in 4T1 was across the entire landscape in most tumors with stromal restriction in 30%. We also performed spatial distribution studies by using mIHC analysis. The same T-cell distribution was seen with multiplex immunohistochemistry (mIHC), where α-SMA staining intensity in the stromal cores, followed the order EMT6>Py8119>EMT6. Data are expressed as mean±SD, n=6.

FIG. 6, panels A-B, show CD8+ T-cell spatial distribution landscapes in 4T1, EMT6 and Py8119 TNBC animal models (Nel et al. (2022), ACS Nano. 16: 5184-5232). 4T1 (Balb/c), EMT6 (Balb/c) and Py8119 (C57BL/6) EC cells were orthotopically implanted in mouse mammary pads on day 0. When the tumors reached 100-150 mm3, animals were IV injected on days 8, 11 and 14 with DOX-NP (5 mg/kg; Avanti Polar Lipids) or left untreated (UT). Panel A) Tumors were collected on day 21 and analyzed by multiplex IHC (mIHC) staining. Panel B) For mIHC analysis, tumor sections were stained with primary antibodies: CD8, α-SMA and Ki-67. Quantitative analysis of CD8+ numbers in cores and margins was performed using Akoya InForm Image Analysis software. Doxorubicin treatment induced increased CD8+ T-cell recruitment in all tumor types with both staining methods. Importantly, newly recruited CD8 T-cells tended to be margin- or stroma-restricted in EMT6 and Py8119 tumors, while CTL distribution in 4T1 was across the entire landscape in most tumors with stromal restriction in 30%. We also performed spatial distribution studies by using mIHC analysis. The same T-cell distribution was seen with mIHC, where α-SMA staining intensity in the stromal cores, followed the order EMT6>Py8119>EMT6. Data are expressed as mean±SD, n=6.

FIG. 7 shows the impact a CXCR4 inhibitor in breast cancer. A number of preclinical studies support the use of CXCR4 antagonists for sensitization to chemotherapy and immune checkpoint inhibitors (ICIs) in solid cancers. This includes the work of Chen et al. (2019) Proc. Natl. Acad. Sci. USA, 116(10): 4558-4566) who used Plexifor (AMD-3100) with success in metastatic BC models (including 4T1) to demonstrate that targeting CXCR4/CXCL12 signaling leads to a decrease in desmoplasia, interference in metastatic spread, imp rove blood perfusion, increased CTL recruitment, and decreased immunosuppression in both primary and metastatic breast cancer lesions. As shown in this figure utilizing another CXCR4 inhibitor (AMD11070), we also showed interference in T cell exclusion in the margin of 4T1 breast cancer tumors during performance of immunohistochemistry.

FIG. 8 illustrates the selection of CXCR4 antagonists that can be used for remote loading of silicasomes and liposomes. These antagonists were chosen based on potency, size, solubility and pKa values, predicting the possibility of remote loading by protonating agents. Plerixafor (AMD 3100) is an immunostimulant used for mobilizing hematopoietic stem cells from the bone marrow into the bloodstream in cancer patients. The stem cells are then extracted from the blood and transplanted back to the patient. The drug was developed by AnorMED, which was subsequently bought by Genzyme. Mavorixafor (AMD-11070) is a small molecule drug candidate that belongs to a new investigational class of anti-HIV drugs known as entry (fusion) inhibitors. AMD-11070 has been studied in Phase I/II clinical trials for the treatment of Renal cell carcinoma and Phase I clinical trials for the treatment of malignant melanoma and solid tumors. AMD3465 is a potent antagonist of CXCR4, and potently inhibits the replication of X4 HIV strains (IC50: 1-10 nM). All three drugs show a solubility of >2.5 mg/mL at pH 7.

FIG. 9 illustrates a protocol for liposome construction for remote loading of CXCR4 antagonists.

FIG. 10 shows the size, polydispersity index (PDI), zeta potential, and loading capacity for 3 CXCR4 antagonist liposomes.

FIG. 11 shows the shows the physicochemical characterization of a silicasome in which sucrose sulfate was used, to accomplish the loading capacity for AMD11070 of 20%. the particle size was 132.7±1.0 nm, PDI 0.074±0.028′ and Zeta potential −8.97 mV.

FIG. 12, panels A-C, show that nano-formulated CXCR4 inhibitors suppress tumor metastases overcome exclusion of CD8+ cytotoxic T cells in the orthotopic 4T1 TNBC tumor model (see, e.g., Nel et al. (2022) ACS Nano. 165184-5232). Orthotopic 4T1 tumors were established as described in FIGS. 2-4. These animals develop a high rate of lung metastasis. Panel A) Liposomal Doxorubicin (DOX-NP) induces significant 4T1 shrinkage (bottom left panel), with evidence of an immunogenic response as shown in above, in FIGS. 2-4. Panel B) Free AMD11070 alone also leads to tumor shrinkage, which was significantly enhanced when combined with DOX-NP. Panel C) IVIS imaging of explanted animal lungs also demonstrates significant metastasis reduction under all conditions where AMD11070 was used.

FIG. 13, panels A-B, shows spaghetti plot growth curves for the tumors depicted in FIG. 12 (panel A) and CD8 spatial distribution (panel B). In addition, combination therapy with DOX-NP plus liposomal AMD11070 provided additional tumor size reduction, in addition to accomplishing the highest CTL recruitment to the tumor core for CTLs excluded in the margin. The IVIS imaging (panel B) of explanted animal lungs in FIG. 12 also demonstrated significant metastasis reduction under all conditions where AMD11070 was used.

FIG. 14 illustrates tumor size in various treatment regimens in an orthotopic EMT6 breast cancer model.

FIG. 15 shows spaghetti plots of tumor size for the various treatments shown in FIG. 14.

FIG. 16, panels A-B, shows tumor size (panel A) and tumor spatial distribution (panel B) of CD8+ cells in various treatment regimes in an orthotopic EMT6 breast cancer model.

FIG. 17, panels A-B, illustrate pharmacokinetics and tumor growth using a silicasome carrier for AMD11070 in an orthotopic KPC model. Panel A) AMD11070-silicasomes were used to perform a PK study in 10-12-week-old female B6/129SF1/J mice bearing KPC tumors. The animals received IV injection of free AMD11070 or AMD11070-silicasome at a drug dose of 5 mg/kg, followed by collection of blood samples at 5 min, 1, 4, 24, and 48 hrs. After separation of the plasma fraction, the drug was extracted in an acidic methanol solution (0.1 mol/L phosphoric acid/methanol, 1:4 v/v). Drug content in the tumor tissue was obtained from KPC tumor bearing animals 24 hr after drug administration. The PK data were analyzed by PKSolver software, using a one-compartment model. Panel B) We also investigated treatment impact on tumor weight and the CD8/Treg ratio in orthotopic KPC tumor bearing mice, as shown in the bottom right panel. Animals received 3 IV administrations on days 8, 11 and 14, using the formulations shown in the legend, or were left untreated. Animals were sacrificed on day 17. Orthotopic tumors were collected, weighed, and prepared for sectioning to perform mIHC analysis, as described in FIG. 5. While tumor growth inhibition by IRIN was not increased by co-delivery of the AMD11070-silicasome, this treatment resulted in a significant increase in the CD8/FoxP3 ratio in combination treatment with a Irinotecan silicasome, as demonstrated in the IHC analysis in FIG. 18. * p<0.05 compared to saline.

FIG. 18. Immunohistochemistry (IHC) analysis of the animals described in FIG. 17.

FIG. 19 shows the results of an immunohistochemistry analysis to assess CXCR4 expression in the KPC model. Immunohistochemistry staining was performed using a fluorescent labeled antibody to CXCR4. CXCR4 is expressed on the cell surface of most leukocytes, including B cells, and monocytes and most T lymphocyte subsets, but just weakly on NK cells. It is also expressed on nonhematopoietic cells such as endothelial cells and epithelial cells. The results demonstrate that encapsulated but not free AMD11070 delivery could significantly increase CXCR4 staining intensity in the orthotopic tumors. While the exact cell types showing increased staining was not identified, the data is in keeping with increased AMD11070 delivery to the tumor site when incorporated in the silica silicasome.

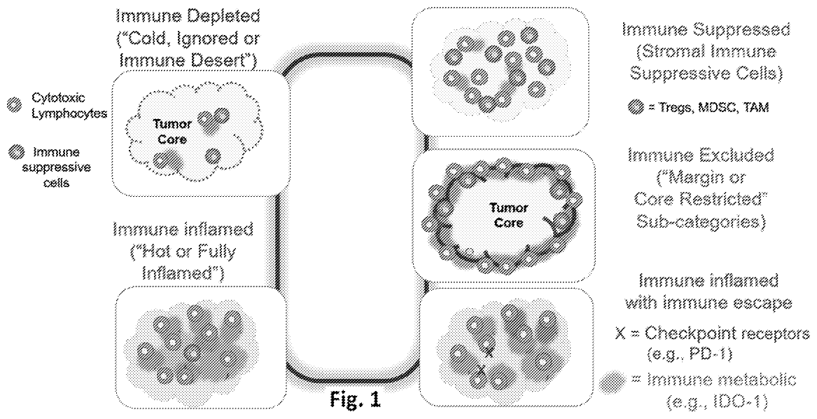

### DETAILED DESCRIPTION

This disclosure pertains to an investigation as to whether intratumoral heterogeneity can be therapeutically targeted or exploited to improve combination therapy, including the use of CXCR4 antagonists, in the era of immune checkpoint blockers. Not only does this require knowledge about the makeup of heterogeneous tumor landscapes, but can also provide a rational approach for combining active pharmaceutical ingredients (API) to reprogram “cold” immune landscapes, overcome T-cell exclusion, overcome T-cell exhaustion by checkpoint receptors, circumvent IDO-1 suppression, and address the immune suppressive properties of the tumor stroma. While currently these challenges are being addressed by theory, immune phenotypin, and modeling, it is a challenge to replicate the complexity of the TIME outside the human body, leading to the conceptualization of therapeutic combinations beyond the number of study subjects that are available to conduct these studies. This prompted us to consider whether preclinical animal models can be used to therapeutically address the heterogeneous TME of pancreatic ductal adenocarcinoma (PDAC) and triple negative breast cancer (TNBC), including intervention with custom-designed nanocarriers or drug delivery systems (DDS).

We have developed nanoprobes to develop combination therapy with lipid bilayer (LB)-coated nanocarriers, that can provide combination therapies. Here, we demonstrate the use of the lipid bilayer for remote drug loading of drug agents that interfere in T-cell exclusion into liposomes and silicasomes.

The complexity of the tumor microenvironment (TME) resulting from the collective contribution of tumor cells, infiltrating immune cells, fibroblasts, tumor vasculature and extracellular matrix determines the success of solid tumor immunotherapy, including pancreatic, breast, colorectal, lung, melanoma, and head and neck cancers (see, e.g., Oliver et al. (2018) Front. Immunol. doi.org/10.3389/fimmu.2018.00070; Vitale et al. (2021) Nat. Med. 27(2): 212-224; Ho et al. (2020) Nat. Rev. Clin. Oncol. 17(9): 527-540). This includes the impact on immunotherapy, where a major objective is to improve the response in cancers such as pancreatic ductal adenocarcinoma (PDAC) and triple-negative breast cancer (TNBC). Not only are PDAC and TNBC two of the most formidable cancers that we encounter in oncology, but also present poorly immunogenic landscapes that resist immunotherapy. PDAC is the fourth leading cause of cancer deaths in the United States, with median survival less than 6 months or a 5-year survival rate in the single digit range (Korc (2007) Am. J. Surg. 194: S84-S86; Stark et al. (2016) Surgery, 159: 1520-1527). Moreover, this cancer is typically diagnosed at an advanced stage, which precludes surgery and is poorly responsive to chemotherapy or immunotherapy, including treatment with checkpoint blocking antibodies. Similarly, TNBC is considered one of the highest-risk breast cancer subtypes, with a local recurrence rate >70% within 5 years or a 5-year survival rate of 12% for metastatic disease (Berger et al. (2021) Pharmaceuticals, 14: 763).

The variable composition of the TME, comprised of tumor cells, vasculature, extracellular matrix, fibroblasts and infiltrating immune cells contributing to the establishment of heterogeneous immune landscapes, is of major interest for PDAC, TNBC, melanoma, and non-small cell lung cancer (see, e.g., Nel et al. (2022) ACS Nano. 16: 5184-5232). This has given rise to the development of integrated approaches for immune and molecular-directed therapies, making use of new clinicopathologic, genomic/transcriptomic, immunophenotypic and spatial distribution technologies for disease classification and stratification. This discovery is now being used for the development of customized therapy for receptive subgroups to improve immune checkpoint response rates as well as prevent unnecessary treatment in patients not likely to respond. Our focus is on addressing heterogeneous immune landscapes in preclinical PDAC and TNBC animal models, making creative use of LB-coated nanocarriers for the delivery of immunogenic stimuli that can be propagated by co-delivered immune modulatory agents (Id.).

TNBC represents a PC subgroup characterized by a lack of estrogen receptors, progesterone receptors and human epidermal growth factor receptor 2 (HER2). These tumors, representative of 15-20% of BCs, are more prevalent in younger African and Hispanic women and have high rates of distant recurrence, with reduced overall survival. Conventional immune phenotyping of epithelial and stromal compartments has been used to identify TNBC subtypes with gene-based meta-signatures (Gruosso et al. (2019) J. Clin. Invest. 129: 1785-1800). These TIMES were characterized as “Immune desert”, “Fully inflamed”, “Margin-restricted” or “Stroma-restricted” phenotypes. Each subtype represents a significant fraction of human TNBC cases, has prognostic significance and provides therapeutic guidelines. Using CD8+ T-cell density in the tumor cores, these landscapes were initially identified as corCD8-HIGH and corCD8-LOW categories, which were further sub-classified for CD8+ accumulation in the tumor margins (marCD8hi). Most tumors in the corCD8-LOW category showed some CD8+ T-cell accumulation in the tumor margins (marCD8hi) and were designated as a “margin-restricted” (MR) landscape. In contrast, a smaller number of tumor landscapes showed low-density T-cell infiltration everywhere and these were designated as an “immune desert” (ID) phenotype. Similarly, the corCD8-HIGH category was subdivided into “fully inflamed” (FI), with high density CD8+ infiltration in the epithelial and stromal compartments, or “stromal-restricted” (SR) if the CD8+ T-cell accumulation occurred in the stroma but not the epithelial compartment.

In order to perform preclinical studies on heterogeneous immune landscapes that mimic the human findings, we performed orthotopic implants of 4T1, EMT6 and Py8119 cell lines in the mammary pads of syngeneic mice. The characteristics of these TNBC mimicking tumors are described in Table 1.

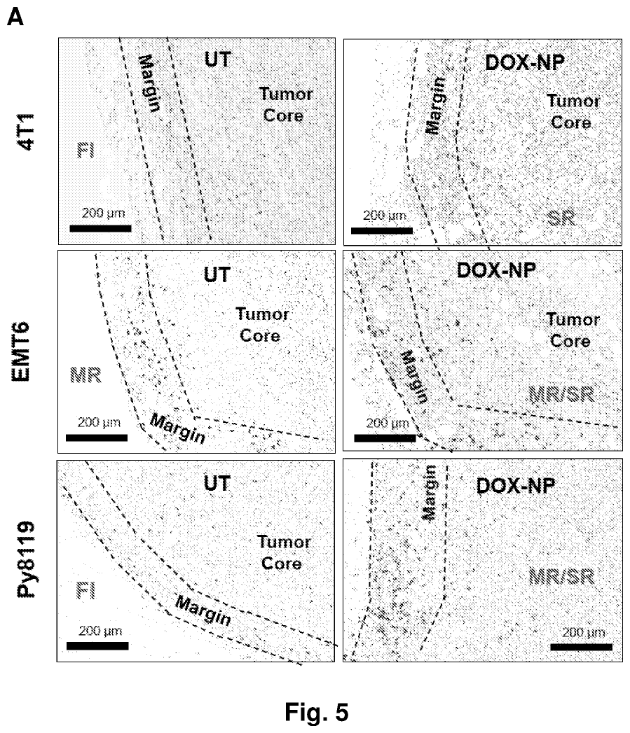

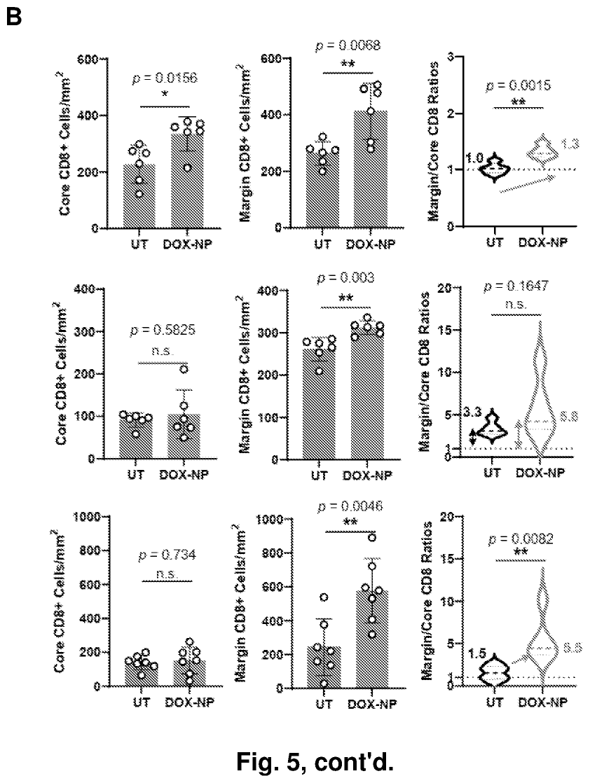

TABLE 1
Murine TNBC models (see, e.g., Nel et al. (2022) ACS Nano. 16: 5184-5232).

| Model | 4T1 | EMT6 | Py8119 |
|---|---|---|---|
| Mouse Strain | Balb/c | Balb/c | C57BL/6 |
| HR | ER/PR/HER2- | ER/PR/HER2- | ER/PR/HER2- |
| Expression | negative | negative | negative |
| Origin | Spontaneous | Derived from a trans- | Cell clone from |
| | mammary intraductal | planted hyperplastic | transgenic tumor, |
| | tumor from Balb/c | alveolar nodule in | where a mouse |
| | | Balb/c | mammary tumor virus |
| | | | promoter drives |
| | | | polyoma middle T |
| | | | antigen (MMTV- |
| | | | PyMY) |
| Tumor | Aggressive orthotopic | Less invasive | Orthotopic Py8119 |
| Growth, | model that exhibits | orthotopic model, with | tumors do not |
| Metastasis | characteristics of | no or limited | metastasize (different |
| and | human basal-like | metastatic potential, | from the transgenic |
| Immunity | TNBC, with high | generally regarded as | model) but can |
| | metastatic potential, | moderately | establish lung |
| | including to lymph | immunogenic with | metastasis after IV |
| | nodes, blood, liver, | partial responsiveness | injection. Generally |
| | lung, brain, and bone. | to anti-PD1 and anti- | regarded as a poorly |
| | Relatively high level | CTLA4 but could | immunogenic model |
| | of tumor inflammation | response to both. The | and refractory to anti- |
| | with abundant | EMT6 cells express | PD1, anti-CTLA4 or |
| | cytokine and | high levels of | combination therapy. |
| | chemokine production, | chemokines and | Successful study of |
| | as well as recruitment | cytokines, MHC-I, and | radiation-induced |
| | of activated CTLs, B- | antigen presentation | immune responses. |
| | cells and PD-1 | potential, compared to | Oxidized cell lysates |
| | expression. | all other orthotopic | delivered in a |
| | Granulocytic-MDSC | models. Tumors show | liposomal spherical |
| | facilitates metastatic | less recruitment of | nucleic acid |
| | spread at pre- | granulocytic-MDSC, | (adjuvant), highly |
| | metastatic niches. | adoptive transfer of | immunogenic. |
| | Generally regarded as | the cells from 4T1 | |
| | poorly immunogenic, | mice increases | |
| | with resistance to anti- | metastatic potential. | |
| | PD1, anti-CTLA4 as | No metastasis after | |
| | well as combination | tumor resection, good | |
| | therapy. Metastasis | immune memory | |
| | occur after primary | prevents secondary | |
| | treatment resection, | challenge. | |
| | with poor immune | | |
| | memory. | | |
| TME and | Fibrotic stroma in | CD3⁺ cells | Py8119 tumors |
| Spatial | which CXCR4 plays a | predominantly | express a fibrotic |
| Distribution | role in T-cell | confined to the | stroma, with high |
| | exclusion, Treg | invasive margin in | levels of mesenchymal |
| | recruitment, immune | untreated tumors. T- | markers and a |
| | suppression, and | cell exclusion | prominent TGF-β |
| | metastatic potential. | successfully blocked | signature. |
| | CXCR4 inhibitors | by anti-TGFβ antibody | |
| | synergize with ICI's in | plus anti-PD-L1 | |
| | limiting desmoplasia | | |
| | and metastases. | | |
| Spatial | Fully inflamed | Margin-localized CTL | The untreated tumor is |
| Distribution | phenotype, with | distribution in | fully inflamed (with |
| (FIG. 5) | increased CTL | untreated and | less dense CTL |
| | recruitment during | Doxorubicin treated | infiltration than 4T1), |
| | Doxorubicin | tumors; tumors exhibit | but assume a margin |
| | treatment, where cells | denser stroma than | and stromal restricted |
| | remain widely | 4T1. | distribution with |
| | distributed or may | | Doxorubicin |
| | become stromal | | treatment. |
| | restricted | | |

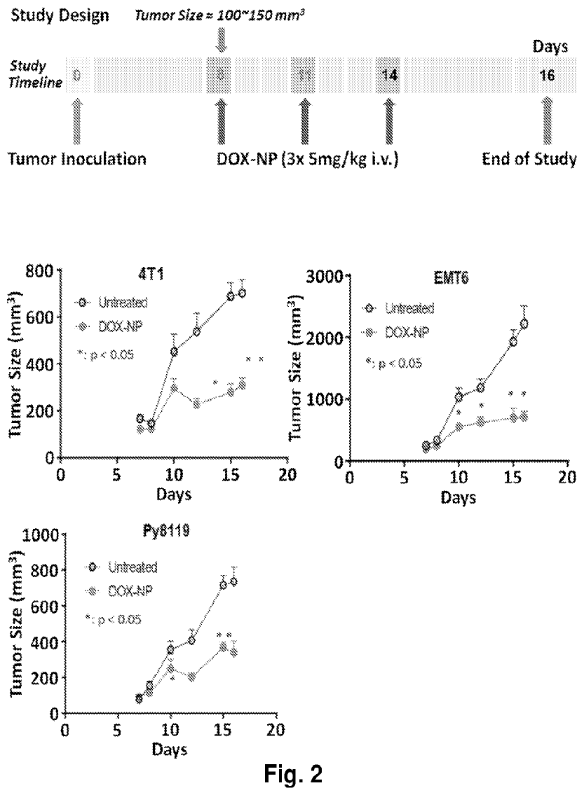

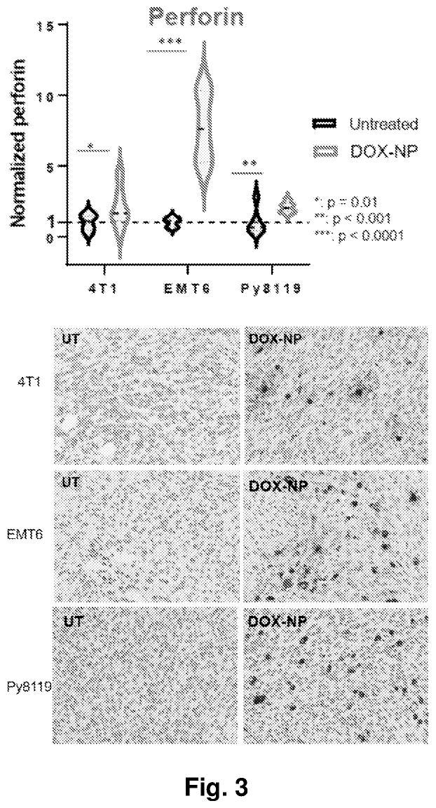

These preclinical models were used to assess the spatial distribution of CD8+ CTLs, using conventional and mIHC analysis under basal growth conditions as well as after the IV injection of a commercial pegylated liposomal Doxorubicin, as outlined in FIGS. 2-4. Doxorubicin is frequently used as neoadjuvant therapy in human TNBC and is a robust inducer of immunogenic cell death (ICD) (see, e.g., Fucikova et al. (2020) Cell Death Dis. 11: 1013; Janicka et al. (2017) Expert Opin. Drug Deliv. 14: 1059-1075). FIG. 5 depicts the spatial distribution of CD8+ T-cells in 4T1, EMT6 and PY8199 tumor landscapes, using conventional IHC staining. The data demonstrate increased CD8+ density with Doxorubicin treatment in all the landscapes, but with differences in the spatial distribution of newly recruited T-cells. Thus, while in untreated animals, 4T1 tumors tend to be fully inflamed, the recruitment of additional CD8+ T-cells during Doxorubicin treatment remained widely distributed, except for stromal-restricted profiles in 30% of cases. The visual impression was confirmed by quantification of CD8+ cell numbers (cells/mm2) in the margins and cores (upper panel). In contrast, the margin-restricted basal landscape in EMT6 tumors maintained the same distribution under basal and treated conditions, except for the Doxorubicin-induced increase in CTL density (middle panel). Also, the fully inflamed phenotype of PY8199 tumors, reverted to a margin- or stromal-restricted distribution pattern upon treatment with Doxorubicin, which increased T-cell density (FIG. 5, lower panel).

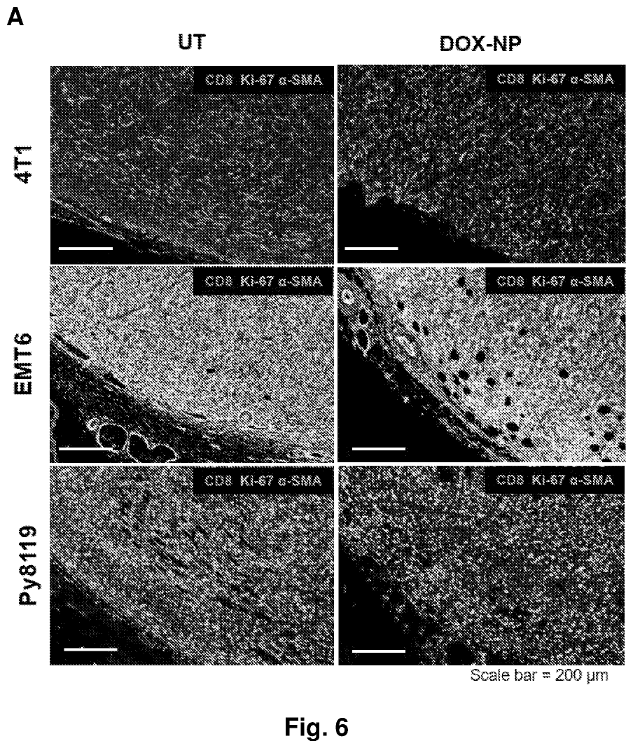

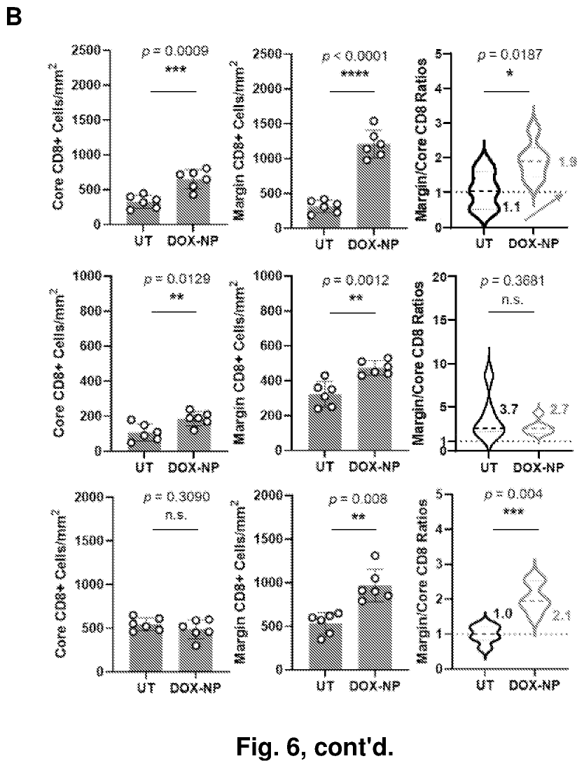

Tumor slices from the same animals were used for multiplex immunohistochemistry (mIHC) analysis, using OPAL reagents (Akoya Biosciences) to obtain spectrally mixed images for CD8, Ki-67, αSMA, Foxp3, CD68/CD163 and DAPI, as described in FIG. 6. Spectral unmixing to focus on CD8, Ki-67 and αSMA confirmed that liposomal Doxorubicin increased the density of CD8+ cells (red fluorescence), which were spatially distributed in similar manner as conventional IHC for each tumor type (FIG. 5). Moreover, α-SMA staining indicated inter-tumor stromal differences, such that the fluorescence staining intensity for EMT6 tumors was >PY8119>4T1. All considered, the data in FIG. 5 allowed the grouping of the murine TNBC landscapes into categories that partially overlap the human landscapes (Table 1). Table 1 summarizes the spatial distribution features together with the distinguishing immunological features of these tumors.

Attempts to study heterogeneous human PDAC immune landscapes in preclinical animal studies are limited. The most frequently studied genetically engineered mouse model is LSL-KrasG12D/+; LSL-Trp53R172H/+; Pdx1-Cre (KPC), which uses Cre-Lox to express activated Kras and a mutant p53 version under the control of a pancreatic epithelial cell-specific Pdx1 or Ptf1a promoter (Muzumdar et al. (2016) Nat. Commun. 7: 12685). These animals develop tumors that mimic several human PDAC characteristics, including a limited number of driver mutations (KRAS, TP53, CDKN2A and SMAD4), also reflected in the human mutational signaling clusters. (Overall, there is a low mutational burden and display of neoepitopes in both human and murine PDAC, both of which show the loss of p53 function during stage-wise progression from pancreatic intraepithelial neoplasia (PanIN) to the stage of full-blown desmoplasia (see, e.g., Muzumdar et al. (2016) Nat. Commun. 7: 12685; Hingorani et al. (2005) Cancer Cell, 7: 469-483; Li et al. (2013) Chin. J. Cancer Res. 25: 715-721). The TME displays considerable heterogeneity as a result of the dependence on diverse EMT cancer pathways. An example of mIHC multispectral imaging of stromal features in the KPC model is shown in Carstens e (2017) Cancer. Nat. Commun. 8: 15095. Additional overlapping histopathological features with human PDAC include poor vascularity and high metastatic burden (see, e.g., Hingorani et al. (2005) Cancer Cell, 7: 469-483; Olive et al. (2006) Cancer Res. 12: 5277-5287). While both cancer types show dense infiltration of immunosuppressive TAMs and myeloid cells, the lesser expression of CTL in murine KPC does not mirror the occasional occurrence of an immune-rich subtype in human PDAC. However, similar to human PDAC, KPC tumor-bearing mice are mostly refractory to ICIs, including administration of anti-PD-L1/PD-1 and anti-CTLA-4 antibodies.

In spite of the differences in CD8+ T-cell and TAMs densities in human and murine PDAC, the spontaneous KPC animal orthotopic model has been useful to develop new PDAC therapies that could be translated in the clinic (see, e.g., Li et al. (2018) Immunity, 49: 178-193.e7; Shibuya et al. (2014) PLOS ONE, 9: e96565). This includes studies looking at the role of CD40, which is broadly expressed in immune cells and capable of mediating tumor regression by macrophages, independent of T cells (see, e.g., Elgueta et al. (2009) Immunological Rev. 229: 152-172; Beatty et al. (2011) Science, 331: 1612-1616). These studies provided the impetus for clinical trials using CD40 agonists in advanced stage PDAC, including accomplishment of significant tumor shrinkage in a phase 1b clinical trial. Immune profiling of these patients demonstrated rapid DC activation as well as reprogramming of the myeloid compartment. Another example of KPC-inspired immunotherapy was the elucidation of the role of the CXCL12/CXCR4 axis in directed migration of immune cells under the instruction of several chemokine receptors (CXCR1, CXCR3, CXCR5, CXCR6, CCR2) (see, e.g., Seo et al. (2019) Clin. Cancer Res. 25: 3934-3945; Feig et al. (2013) Proc. Natl. Acad. Sci. USA, 110: 20212-20217). CXCL12 is produced by CAFs and can attract CXCR4-positive inflammatory, vascular, and stromal cells into the tumor mass to support tumor development (see, e.g., Orimo et al. (2005) Cell, 121: 335-348; Burger et al. (2006) Blood, 107: 1761-1767). Discussed below is the impact of blockade of the CXCL12/CXCR4 axis in improving human PDAC immunotherapy responses to ICI's (Bockorny et al. (2020) Nat. Med. 26: 878-885).

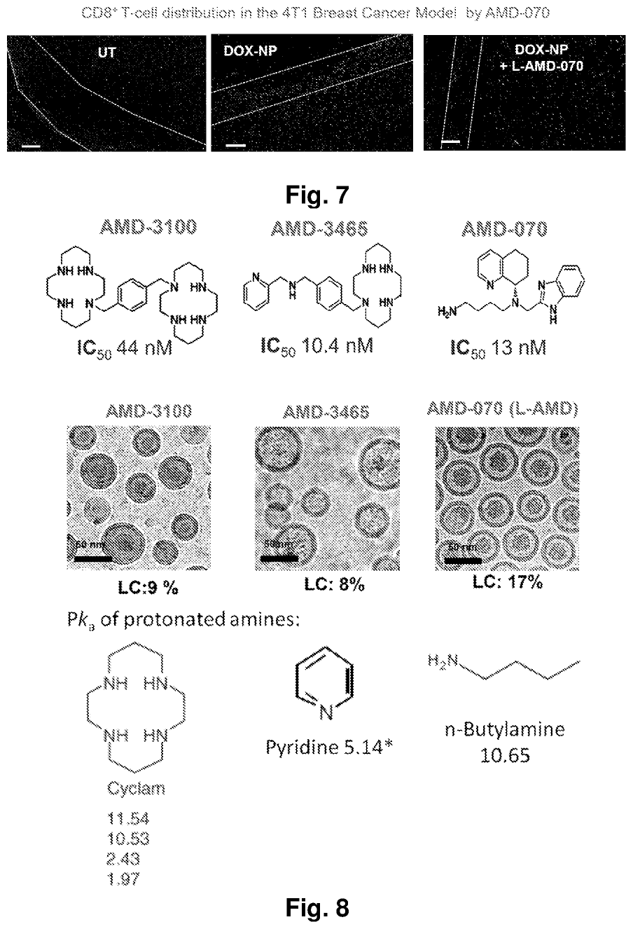

There is growing evidence from preclinical PDAC and TNBC studies that, in addition to the poor cancer immunogenicity, important stromal cell types such as TAFs, MDSCs and TAM) restrain CTL efficacy, in addition to preventing cellular contact with cancer cells (see, e.g., Ho et al. (2020) Nat. Rev. Clin. Oncol. 17(9): 527-540). Among the networks of cellular activation pathways that control the balance between immune attack and immune evasion, the CXCL12/CXCR4 axis is of particular importance for cellular communication in the TME, with influences on tumor vascularization, Treg recruitment, spatial distribution of T-cells, cancer cell proliferation and metastasis (see, e.g., Guo et al. (2016) Oncogene, 35: 816-826; Joyce & Fearon (2015) Science, 348: 74-80). Consequently, CXCL12/CXCR4 overexpression correlates with poor prognosis, including for BC and PDAC (see, e.g., Chen et al. (2019) Proc. Natl. Acad. Sci. USA, 116:4558-4566). It is relevant, therefore, to consider the impact of the CXCL12/CXCR4 axis on the immune suppressive effects of stromal cells in immunotherapy design. This includes the use of SMI such as Plerixafor (AMD3100) and Mavorixafor (AMD11070) or the synthetic peptide, BL-8040, to interfere in CXCL12 binding to CXCR4, with the potential of preventing T-cell exclusion, reducing Treg and MDSC recruitment, and metastasis inhibition (FIGS. 7 and Bockorny et al. (2020) Nat. Med. 26: 878-885; Chen et al. (2019) Proc. Natl. Acad. Sci. USA, 116:4558-4566).

A number of preclinical studies support the use of CXCR4 antagonists for sensitization to chemotherapy and ICIs in solid cancers. This includes the work of Chen et al., who used Plexifor with success in metastatic BC models (including 4T1) to decrease desmoplasia, limit metastatic spread, improve blood perfusion, increase CTL recruitment, and to decrease immunosuppression (FIG. 7) (Chen et al. (2019) Proc. Natl. Acad. Sci. USA, 116:4558-4566). Similarly, the efficacy of BL-8040, was documented in numerous preclinical studies (e.g., melanoma, breast and lung cancers), where CXCR4 blockade could also be seen to mobilize T-cells and NK cells from the bone marrow and lymph nodes to the tumor site (see, e.g., Bockorny et al. (2020) Nat. Med. 26: 878-885; Tamamura et al. 92003) FEBS Letts. 550(1-3): 79-83).

Bockorny et al. used the peptide BL-8040, a CXCR4 antagonist, in combination with pembrolizumab and chemotherapy to treat patients with pancreas cancer in the COMBAT trial (Vitale et al. (2021) Nat. Med. 27(2): 212-224). Patients received 11 cycles of BL-8040 and pembrolizumab. They showed improved survival in combination with checkpoint blocking antibodies in a phase II clinical trial in patients with metastatic PDAC. They also demonstrated increased CTL infiltration, cytotoxic killing and shrinkage of the ductal cancerous structures. Additional treatment responses included decreased densities of granulocytic-MDSCs and circulating Tregs (CD4⁺/CD25⁺/FoxP3⁺ cells).

Seo et al. also demonstrated that CXCR4 blockade with AMD3100 could improve CTL migration to the juxta-tumoral PDAC compartment in a time sequence multicolor fluorescence study in tumor cell slices (Seo et al. (2019) Clin. Cancer Res. 25: 3934-3945). Seo et al. used a combination of mIHC plus TCR deep sequencing to demonstrate the presence of clonally expanded CD80 T-cell populations in human PDAC slices (Seo et al. (2019) Clin. Cancer Res. 25: 3934-3945). Time-lapse confocal microscopy was performed during live t imaging tumor in a tumor slice culture system to track CD8⁺ T-cells, EpCAM (epithelial cellular adhesion molecule) cancer cells and fibroblasts (stain for fibroblast activation protein), prior to treatment, as well as following the addition of AMD3100 (CXCR4 antagonist), anti-PD-1 only or combination of AMD3100 plus anti-PD-1. Notably, they demonstrated that the treatment combination mediates CD8⁺ T-cell-mediated elimination of EpCAM⁺ PDAC cancer cells, accompanied by stromal reduction. Monotherapy did not exert similar effects.

BL-8040 was also used to show improved survival in combination with checkpoint blocking antibodies in a phase II clinical trial in patients with metastatic PDAC (Bockorny et al. (2020) Nat. Med. 26: 878-885). The response was characterized by increased CD8+ T-cell infiltration, while MDSC and Treg numbers are suppressed. Seo et al. also demonstrated that CXCR4 blockade with AMD3100 could improve CTL migration to the juxta-tumoral PDAC compartment in a time sequence multicolor fluorescence study in tumor cell slices (see, e.g., Seo et al. (2019) Clin. Cancer Res. 25: 3934-3945).

While CXCR4 antagonists are clinically approved for mobilizing hematopoietic bone marrow precursors, their impact overcoming T-cell exclusion and recruitment of immunosuppressive cells in solid tumors has resulted in implementation of new combination immunotherapies that include the use of nanocarriers drug delivery systems (DDS). One approach has been the attachment of AMD3100 to pegylated nab-Paclitaxel nanoparticles, leading to improved outcomes in ovarian cancer (Xue et al. (2020) Int. J. Nanomedicine, 15: 5701-5718).

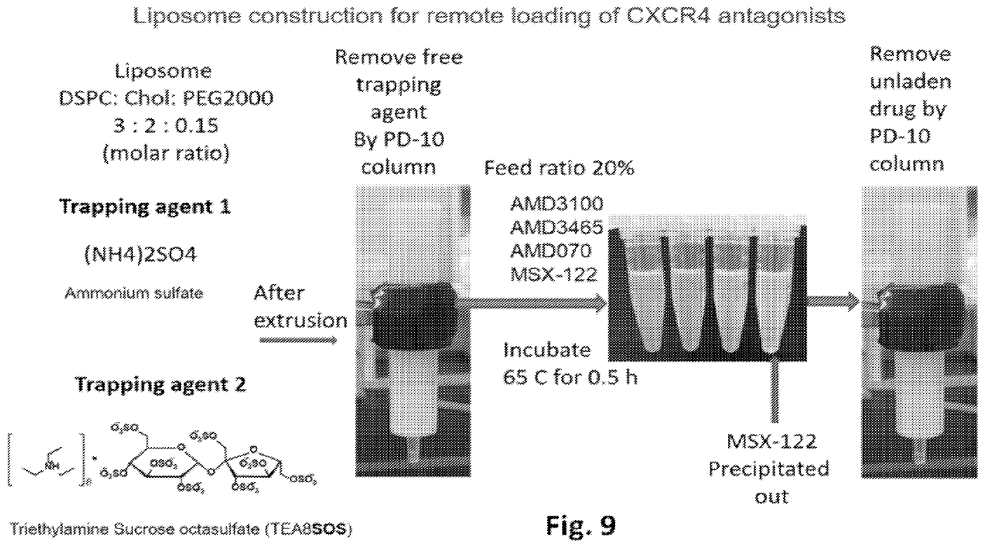

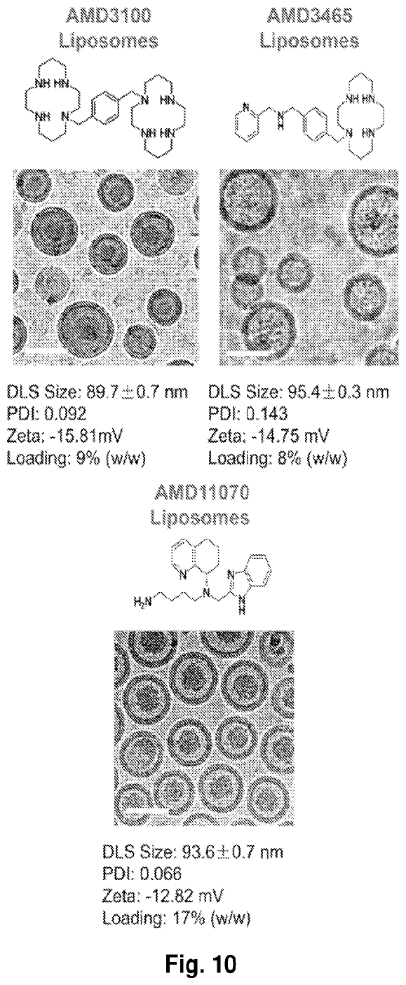

In contrast, our approach has been to determine if the weak basic properties of AMD3100, AMD3465 and AMD11070 could be used to achieve remote loading in LB carriers, using TEA8SOS or ammonium sulfate trapping agents (FIG. 8). These drugs were chosen for their inhibitory potency of the activity of the CXCR4 chemokine receptor, size, solubility and pKa values, predicting the possibility of remote loading by the protonating agents such as sucrose sulfate and ammonium sulfate (FIG. 9). The best loading capacity (LC) in liposomes was for AMD11070 (LC=17%), prompting construction of an AMD11070-silicasome (FIG. 9-10 and Table 5).

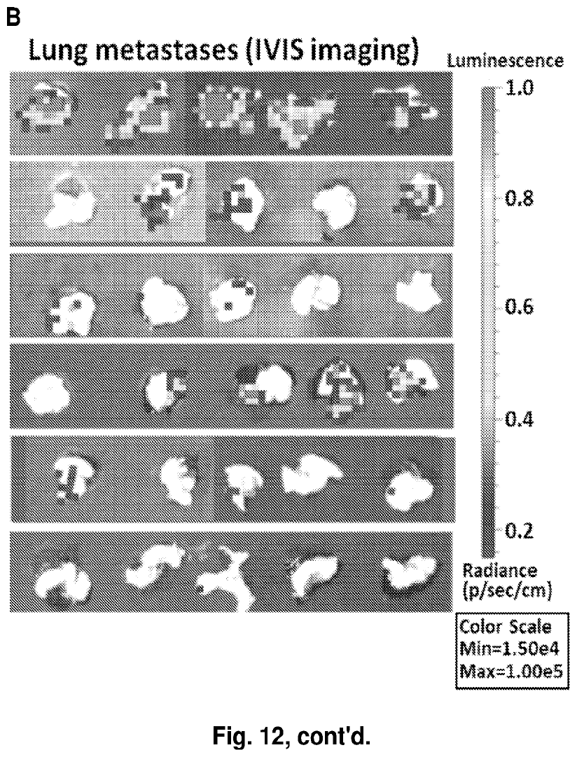

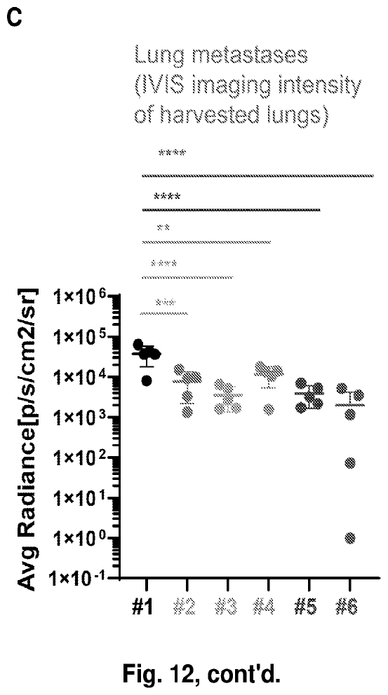

The first animal study with the AMD11070-liposome was to assess the impact of combination therapy with a liposome delivering Doxorubicin in orthotopic 4T1 and EMT6 tumor models (FIGS. 12-16). Following same-day IV administration of both carriers, tumor shrinkage could be obtained in 4T1 tumors using free AMD11070 alone, liposomal L-AMD11070, DOX-NP®, DOX-NP® plus free AMD11070, and DOX-NP® plus L-AMD11070 (see, Example 1). The latter treatment combination resulted in the most significant tumor reduction, with evidence of improved cytotoxic killing. While both free and encapsulated AMD11070 failed to show a significant effect on CD8+ spatial distribution during mIHC analysis, both treatments had a significant impact on lung metastases, in keeping with the important role of CXCR4 in this disease setting (see, Example 1). Similar experimentation in EMT6 demonstrated that co-administration of encapsulated Doxorubicin and AMD11070 resulted in significant tumor shrinkage. Encapsulated AMD11070 was effective in allowing more abundant CTL recruitment to the tumor core (see Example 1).

The AMD11070-silicasome was used to investigate the impact on drug biodistribution and immunogenic effects in the murine orthotopic KPC model. Co-delivery of the AMD11070 silicasome with IRIN-silicasome+AMD11070 resulted in a significant increase in the CD8/FoxP3 ratio IRIN-silicasome+AMD11070.

Accordingly, in various embodiments, a drug delivery vehicle is provided where the drug delivery vehicle comprises: 1) a silicasome comprising a mesoporous silica nanoparticle coated with a lipid bilayer and further comprising a CXCR4 antagonist; or 2) a liposome comprising a lipid bilayer comprising where said liposome further comprises a CXCR4 antagonist. Methods of use of such drug delivery vehicles are also provided. Moreover, to complement above results, a number of our previously described dual-delivery carriers (e.g., silicasomes) can be used to combine CXCR4 antagonists with other therapeutic agents. This includes in certain embodiments, LB-coated carriers with remote loading of AMD11070 in addition to the encapsulation of, for example, an IDO inhibitor (e.g., indoximod) and/or immune checkpoint inhibitor (ICI) prodrugs in the lipid membrane. CXCR4 antagonists can also be anchored to a component of the lipid bilayer (e.g., to PEG that is incorporated in a LB) of the silicasomes or liposomes that are also used for loading immunogenic cell death (ICD)-inducing drugs and/or GSK3 inhibitors. This topological arrangement also allows the conjugated drug to target CXCR4 expressing tumor or metastatic sites. Examples of customized, nano-enabled combination therapy for TNBC, including use of CXCR4 antagonist appear in Table 6.

Drugs Loaded into or Conjugated onto the CXCR4 Antagonist Liposome or Silicasome.

As explained above, in various embodiments, a drug delivery vehicle is provided where the drug delivery vehicle comprises: 1) a silicasome comprising a mesoporous silica nanoparticle coated with a lipid bilayer and further comprising a CXCR4 antagonist; or 2) a liposome comprising a lipid bilayer comprising where said liposome further comprises a CXCR4 antagonist. CXCR4 antagonists are well known to those of skill in the art. Illustrative CXCR4 antagonists include, but are not limited to Plerixafor (AMD3100), Mavorixafor (AMD11070), AMD3465, the synthetic peptide, BL-8040, and the like.

In certain embodiments the drug delivery vehicles can incorporate one or more drugs in addition to the CXCR4 antagonist(s). In certain embodiments such additional drugs include, but are not limited to IDO inhibitors, immune checkpoint inhibitors (ICIs), immunogenic cell death-(ICD)-inducing drugs, GSK3 inhibitors, and the like.

### IDO-1 Inhibitors

In certain embodiments the additional drugs include, but are not limited to IDO-1 inhibitors (inhibitors of the IDO pathway) such as D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-β-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide, N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, 4-phenylimidazole, and the like).

### Immune Checkpoint Inhibitors (ICIs)

In certain embodiments the additional drugs include but are not limited to one or more immune checkpoint inhibitors (ICIs). Illustrative immune checkpoint inhibitors include, but are not limited to to inhibitors of PD-1, PD-L1, PD-L2, PD-L3, PD-L4, CTLA-4, LAG3, B7-H3, B7-H4, KIR and/or TIM3 receptors.

In some embodiments, the immune checkpoint inhibitor can be a small peptide agent that can inhibit regulatory T cell function, including any one or a combination of the inhibitory receptors listed above. In some embodiments, the immune checkpoint inhibitor can be a small molecule (e.g., less than 500 Daltons) that can inhibit T regulatory cell function. including the immune checkpoint receptors listed above. In some embodiments, the immune checkpoint inhibitor can be a molecule providing co-stimulation of T-cell activation. In some embodiments, the immune checkpoint inhibitor can be a molecule providing co-stimulation of natural killer cell activation. In some embodiments, the immune checkpoint inhibitor can be an antibody. In some embodiments, the immune checkpoint inhibitor is a PD-1 antibody. In some embodiments, the immune checkpoint inhibitor is a PD-L1 antibody. In some embodiments, the immune checkpoint inhibitor is a PD-L2 antibody. In some embodiments, the immune checkpoint inhibitor is a PD-L3 antibody. In some embodiments, the immune checkpoint inhibitor is a PD-L4 antibody. In some embodiments, the immune checkpoint inhibitor is a CTLA-4 antibody. In some embodiments, the immune checkpoint inhibitor is an antibody of CTLA-4, LAG3, B7-H3, B7-H4, KIR, or TIM3.

In certain embodiments the antibody can be selected from α-CD3-APC, α-CD3-APC-H7, α-CD4-ECD, α-CD4-PB, α-CD8-PE-Cy7, α-CD-8-PerCP-Cy5.5, α-CD11c-APC, α-CD11b-PE-Cy7, α-CDI1b-AF700, α-CD14-FITC, α-CD16-PB, α-CD19-AF780, α-CD19-AF700, α-CD20-PO, α-CD25-PE-Cy7, α-CD40-APC, α-CD45-Biotin, Streptavidin-BV605, α-CD62L-ECD, α-CD69-APC-Cy7, α-CD80-FITC, α-CD83-Biotin, Streptavidin-PE-Cy7, α-CD86-PE-Cy7, α-CD86-PE, α-CD123-PE, α-CD154-PE, α-CD161-PE, α-CTLA4-PE-Cy7, α-FoxP3-AF488 (clone 259D), IgG1-isotype-AF488, α-ICOS (CD278)-PE, α-HLA-A2-PE, α-HLA-DR-PB, α-HLA-DR-PerCPCy5.5, α-PD1-APC, VISTA, co-stimulatory molecule OX40, CD137, and the like.

In general, PD-1 inhibitors are well known to those of skill in the art. Such inhibitors include, but are not limited to Pembrolizumab, Nivolumab (Opdivo), Cemiplimab (Libtayo), Spartalizumab (PDR001), Camrelizumab (SHR1210), Sintilimab (IBI308), Tislelizumab (BGB-A317), Toripalimab (JS 001), Dostarlimab, INCMGA00012 (MGA012), AMP-224, AMP-514 (MEDI0680), and the like.

PD-L1 inhibitors are also well known to those of skill in the art. Illustrative PD-L1 inhibitors include, but are not limited to Atezolizumab, Avelumab, Durvalumab, KN035, CK-301 by Checkpoint Therapeutics, AUNP12, CA-170, and BMS-986189, and the like. Atezolizumab (Tecentriq) is a fully humanised IgG1 (immunoglobulin 1) antibody developed by Roche Genentech. Avelumab (Bavencio) is a fully human IgG1 antibody developed by Merck Serono and Pfizer. Durvalumab (Imfinzi) is a fully human IgG1 antibody developed by AstraZeneca. KN035 is a PD-L1 antibody with subcutaneous formulation currently under clinical evaluations. CK-301 is an anti-PD-L1 antibody by Checkpoint Therapeutics.

Similarly, CTLA-4 inhibitors are well known to those of skill in the art. One example is Ipilimumab (Yervoy). Additionally, a number of anti-CTLA4 antibodies are described for example in U.S. Patent Publication Nos: US 2020/0206346, US 2019/0276542, US 2019/0241662, US 2019/0225690, US 2019/0185569, US 2019/0177414, US2017/0216433, US 2013/0142805, US 2012/0135001, US 2012/0121604, US 2010/0278828, US 2010/0098701, US 2009/0252741, US 2009/0074787, and US 2008/0152655, which are incorporated herein by reference for the anti-CTLA4 antibodies described therein.

LAG-3 inhibitors are also known to those of skill in the art and include but are not limited to Fianlimab, and Relatlimab which are monoclonal antibodies. Additional LAG-3 inhibitors are described in U.S. Patent Publication No: 2022/0135670 A1.

A number of checkpoint inhibitors are approved for clinical use in the United States by the Food and Drug administration. A list of illustrative FDA approved checkpoint inhibitors is provided in Table 2.

TABLE 2
FDA approved checkpoint inhibitors.

| Name | Target | Approved |
|---|---|---|
| Ipilimumab | CTLA-4 | 2011 |
| Nivolumab | PD-1 | 2014 |
| Pembrolizumab | PD-1 | 2014 |
| Atezolizumab | PD-L1 | 2016 |
| Avelumab | PD-L1 | 2017 |
| Durvalumab | PD-L1 | 2017 |
| Cemiplimab | PD-1 | 2018 |
| Relatlimab | LAG-3 | 2022 |

### Immunogenic Cell Death (ICD)-Inducing Drugs

In certain embodiments the additional drugs include but are not limited to one or more immunogenic cell death (ICD)-inducing drugs. Illustrative ICD-inducing drugs include, but are not limited to doxorubicin, oxaliplatin, anthracenedione, bleomycin, bortezomib, cisplatin, daunorubicin, docetaxel, epirubicin, idarubicin, mitoxanthrone, paclitaxel, R2016, cyclophosphamide, and the like.

### GSK3 Inhibitors.

In certain embodiments the additional drugs include but are not limited to one or more GSK3 inhibitors. GSK3 inhibitors are well known to those of skill in the art, and described, for example, in PCT Application No: PCT/2021/039583 (WO 2022/006083). Illustrative, but non-limiting examples of GSK3 inhibitors include, but are not limited to CHIR9902, tideglusib, SB415286, SB216763, BIO, CHIR98014, AZD2858, TWS119, AZD1080, AR-A014418, 1-azakenapullone, TDZD8, IM-12, LY2090314, 2D0*, indirubin-3-monoxime, BIO-acetoxime, and the like the structures of which are shown in FIG. 10 of PCT Application No: PCT/2021/039583 (WO 2022/006083).

It will be appreciated that in certain embodiments the additional drug(s) are incorporated into the inside of the drug delivery vehicle (e.g., liposome or silicasome). In certain embodiments the additional drug(s) are conjugated to a component of the lipid bilayer (LB) comprising the liposome or silicasomes described herein. Thus, for example, in certain embodiments, the additional drugs are conjugated to a phospholipid, to cholesterol or to a cholesterol derivative, to a pegylated lipid, and so forth. In certain embodiments the additional drug(s) are simply co-administered with the drug delivery vehicles described herein in a free form or in an encapsulated form. Thus, for example, in certain embodiments, doxirubicin can be co-administered as a free form or encapsulated in a separate liposome.

The forgoing drugs and drug combinations are illustrative and non-limiting. Using the teaching provided herein (see, e.g., Table 8), numerous other drugs and drug combinations using the drug delivery vehicles described herein will be available to one of skill in the art.

### Lipid Bilayer Composition of CXCR4 Antagonist Liposomes and Silicasomes.

Among the various embodiments in which drug delivery vehicles are provides there are: (1) a silicasome, comprising a mesoporous nanoparticle (e.g., a mesoporous silica nanoparticle (MSNP) coated with a lipid bilayer and further comprising a CXCR4 antagonist; and (2) a liposome, comprising a lipid bilayer where the liposome further comprises a CXCR4 antagonist.

In various embodiments the lipid bilayer composition of the liposome or silicasome can be optimized to provide a rapid and uniform particle coating, to provide colloidal and circulatory stability, and to provide effective cargo retention, while also permitting a desirable cargo release profile.

In certain embodiments the lipid bilayer comprises a combination of one or more phospholipids, cholesterol and/or a cholesterol derivative (e.g., cholesterol hemisuccinate (CHEMS), lysine-based cholesterol (CHLYS), PEGylated cholesterol (Chol-PEG), and the like), and in certain embodiments, a pegylated lipid (e.g., DSPE-PEG₂₀₀₀), or a functionalized pegylated lipid (e.g., DSPE-PEG₂₀₀₀-maleimide) to facilitate conjugation with targeting or other moieties.

In certain embodiments the lipids used in lipid bilayer of the liposome or silicasome comprise of DSPC:Chol (or CHEMS):DSPE-PEG2000. In certain embodiments he lipids used in lipid bilayer of the liposome or silicasome comprises DSPC:Chol:DSPE-PEG2000 in a molar ratio of 3:2:0.15.

The lipid bilayer formulation(s) described above are illustrative and non-limiting. Depending on the drug(s) being loaded into the liposome or silicasome or conjugated to a component of the lipid bilayer, and the desired release provide, in various embodiments different lipid bilayer formulations can be used and an optimal formulation can be determined.

Accordingly, in certain embodiments the lipid bilayer can comprise: 1) one or more saturated fatty acids with C14-C20 carbon chain, such as dimyristoylphosphatidyl-choline (DMPC), dipalmitoylphosphatidylcholine (DPPC), distearoylphosphatidylcholine (DSPC), and diactylphosphatidylcholine (DAPC); and/or 2) One or more unsaturated fatty acids with a C14-C20 carbon chain, such as 1,2-dimyristoleoyl-sn-glycero-3-phosphocholine, 1,2-dipalmitoleoyl-sn-glycero-3-phosphocholine, 1,2-dioleoyl-sn-glycero-3-phosphocholine (DOPC), 1,2-dieicosenoyl-sn-glycero-3-phosphocholine; and/or 3) Natural lipids comprising a mixture of fatty acids with C12-C20 carbon chain, such as egg PC, and soy PC, sphingomyelin, and 4) a modified cholesterol (e.g., cholesterol hemisuccinate (CHEMS)) or the like. It is noted that, in certain embodiments, in order to compensate a positive charge, it is possible to use cholesteryl hemisuccinate (CHEMS) that carries one negative charge at pH>6.5 in the formulation. These lipids are illustrative but non-limiting and numerous other lipids are known and can be incorporated into a lipid bilayer for formation of a drug delivery nanocarrier (e.g., a bilayer-coated nanoparticle).

In certain embodiments the silicasome contains a lipid (e.g., a phospholipid), cholesterol (or cholesterol derivative), and a PEG functionalized lipid (e.g., an mPEG phospholipid). In certain embodiments the mPEG phospholipids comprise a C14-C18 phospholipid carbon chain from, and a PEG molecular weight from 350-5000 (e.g., MPEG 5000, MPEG 3000, MPEG 2000, MPEG 1000, MPEG 750, MPEG 550, MPEG 350, and the like). In certain embodiments the mPEG phospholipid comprises DSPE-PEG5000, DSPE-PEG3000, DSPE-PEG2000, DSPE-PEG1000, DSPE-PEG750, DSPE-PEG550, or DSPE-PEG350. MPEGs are commercially available (see, e.g., //avantilipids.com/product-category/products/polymers-polymerizable-lipids/mpeg-phospholipids/).

In certain embodiments the lipid bilayer comprises an mPEG phospholipid with a phospholipid C14-C18 carbon chain, and a PEG molecular weight ranging from about 350 Da to 5000 Da. In certain embodiments the lipid bilayer comprises DPSE-PEG_{2K}.

In certain embodiments the lipid bilayer comprises 1,2-distearoyl-sn-glycero-3-phosphoethanolamine-PEG (DSPE-PEG).

In certain embodiments the ratio of phospholipid:Chol:PEG, is about phospholipid (50-90 mol %):Chol (10-50 mol %):PEG (1-10 mol %).

In certain embodiments the liposome or silicasome can comprise a second drug (in addition to the contained CXCR4 antagonist conjugated to a component of the lipid bilayer. In certain embodiments the second drug is conjugated to a moiety such as a lipid (e.g., a phospholipid), vitamin E, cholesterol or cholesterol derivative, and a fatty acid. In various embodiments the second drug is conjugated directly to a component of the lipid bilayer and in other embodiments the second drug is conjugated to a component of the lipid bilayer via a linker (e.g., via a homo-bifunctional or hetero-bifunctional linker). In certain embodiments the linker comprises an HO—(CH₂)ₙ₌₂₋₅—OH linker.

In certain embodiments the liposome or silicasome can comprises a CXCR4 antagonist conjugated to a component of the lipid bilayer. In certain embodiments the CXCR4 antagonist is conjugated to a moiety such as a lipid (e.g., a phospholipid), vitamin E, cholesterol or cholesterol derivative, and a fatty acid. In various embodiments the second drug is conjugated directly to a component of the lipid bilayer and in other embodiments the second drug is conjugated to a component of the lipid bilayer via a linker (e.g., via a homo-bifunctional or hetero-bifunctional linker).

The lipid bilayer formulations provided above are illustrative. As noted, in certain embodiments the lipid composition and molar ratios can be altered, and the drug or drugs can be altered.

In some embodiments, a lipid bilayer comprises a phospholipid, cholesterol, and mPEG phospholipid at a ratio of: 50-90 mol %, 50-60 mol %, 60-70 mol %, 70-80 mol %, 80-90 mol %, 50-70 mol %, 60-80 mol %, or 70-90 mol % phospholipid:10-50 mol %, 10-20 mol %, 20-30 mol %, 30-40 mol %, 40-50 mol %, 10-30 mol %, 20-40 mol %, or 30-50 mol % CHOL:1-10 mol %, 1-3 mol %, 3-6 mol %, 6-10 mol %, 1-5 mol %, 5-10 mol %, 2-8 mol %, 3-7 mol %, or 4-6 mol % mPEG phospholipid.

In some embodiments, the lipid bilayer comprises the phospholipid at a mole percentage of about 50 mol % to about 90 mol %. In some embodiments, the lipid bilayer comprises the phospholipid at a mole percentage of about 50 mol % to about 60 mol %, about 50 mol % to about 70 mol %, about 50 mol % to about 80 mol %, about 50 mol % to about 90 mol %, about 60 mol % to about 70 mol %, about 60 mol % to about 80 mol %, about 60 mol % to about 90 mol %, about 70 mol % to about 80 mol %, about 70 mol % to about 90 mol %, or about 80 mol % to about 90 mol %. In some embodiments, the lipid bilayer comprises the phospholipid at a mole percentage of about 50 mol %, about 60 mol %, about 70 mol %, about 80 mol %, or about 90 mol %. In some embodiments, the lipid bilayer comprises the phospholipid at a mole percentage of at least about 50 mol %, about 60 mol %, about 70 mol %, or about 80 mol %. In some embodiments, the lipid bilayer comprises the phospholipid at a mole percentage of at most about 60 mol %, about 70 mol %, about 80 mol %, or about 90 mol %.

In some embodiments, the lipid bilayer comprises the cholesterol at a mole percentage of about 10 mol % to about 50 mol %. In some embodiments, the lipid bilayer comprises the cholesterol at a mole percentage of about 10 mol % to about 20 mol %, about 10 mol % to about 30 mol %, about 10 mol % to about 40 mol %, about 10 mol % to about 50 mol %, about 20 mol % to about 30 mol %, about 20 mol % to about 40 mol %, about 20 mol % to about 50 mol %, about 30 mol % to about 40 mol %, about 30 mol % to about 50 mol %, or about 40 mol % to about 50 mol %. In some embodiments, the lipid bilayer comprises the cholesterol at a mole percentage of about 10 mol %, about 20 mol %, about 30 mol %, about 40 mol %, or about 50 mol %. In some embodiments, the lipid bilayer comprises the cholesterol at a mole percentage of at least about 10 mol %, about 20 mol %, about 30 mol %, or about 40 mol %. In some embodiments, the lipid bilayer comprises the cholesterol at a mole percentage of at most about 20 mol %, about 30 mol %, about 40 mol %, or about 50 mol %.

In some embodiments, the lipid bilayer comprises the mPEG phospholipid at a mole percentage of about 1 mol % to about 10 mol %. In some embodiments, the lipid bilayer comprises the mPEG phospholipid at a mole percentage of about 1 mol % to about 2 mol %, about 1 mol % to about 4 mol %, about 1 mol % to about 5 mol %, about 1 mol % to about 6 mol %, about 1 mol % to about 8 mol %, about 1 mol % to about 10 mol %, about 2 mol % to about 4 mol %, about 2 mol % to about 5 mol %, about 2 mol % to about 6 mol %, about 2 mol % to about 8 mol %, about 2 mol % to about 10 mol %, about 4 mol % to about 5 mol %, about 4 mol % to about 6 mol %, about 4 mol % to about 8 mol %, about 4 mol % to about 10 mol %, about 5 mol % to about 6 mol %, about 5 mol % to about 8 mol %, about 5 mol % to about 10 mol %, about 6 mol % to about 8 mol %, about 6 mol % to about 10 mol %, or about 8 mol % to about 10 mol %. In some embodiments, the lipid bilayer comprises the mPEG phospholipid at a mole percentage of about 1 mol %, about 2 mol %, about 4 mol %, about 5 mol %, about 6 mol %, about 8 mol %, or about 10 mol %. In some embodiments, the lipid bilayer comprises the mPEG phospholipid at a mole percentage of at least about 1 mol %, about 2 mol %, about 4 mol %, about 5 mol %, about 6 mol %, or about 8 mol %. In some embodiments, the lipid bilayer comprises the mPEG phospholipid at a mole percentage of at most about 2 mol %, about 4 mol %, about 5 mol %, about 6 mol %, about 8 mol %, or about 10 mol %.

In certain embodiments liposome and/or silicasome lipid bilayer formulations can be varied to improve drug-loading capacity (weight of drug/total weight of carrier). In certain embodiments the drug loading capacity is at least about 20%, at least about 30%, or at least about 40%, at least about 50%, at least about 55%, at least about 60%, at least about 65%, at least about 70%, at least about 75%, or at least 80% w/w. In certain embodiments drug loading is greater than 40% w/w, or greater than 45% w/w, or greater than 50% w/w, or greater than 55% w/w, or greater than 60% w/w, or greater than 65% w/w, or greater than 70% w/w, %, or greater than 75% w/w, or greater than 80% w/w.

In certain embodiments the lipid bilayer is formulated to form a substantially uniform and intact bilayer encompassing the entire nanoparticle. In certain embodiments the lipid bilayer is formulated so that the mesoporous silica nanoparticle is colloidally stable.

### Fabrication of Liposomes

The liposomes described herein comprising a CXCR4 antagonist can be fabricated using any convenient method. Liposome fabrication is typically designed to provide a monodisperse population of liposomes with a narrow size distribution and the desired degree of lamellarity, to provide efficient drug inclusion, and to provide long-term colloidal stability of resulting liposomes.

Conventional methods for liposomes preparation involve the following main stages:
1) Dissolution of lipids in an organic solvent;
2) Drying-down of the resultant lipidic solution from the organic solvent;
3) Hydrating the lipid with an aqueous media (followed by agitation/stirring);
4) Downsizing (and/or change in lamellarity);
5) Post-formation processing (purification, sterilization); and
6) Characterization of the final nanoformulation product.

### Thin-Film Hydration (TFH) Method

The thin-film hydration technique (the so-called Bangham method) is the oldest, most common, and simplest method used for the preparation of multi-lamellar vesicles (MLVs). To ensure a homogeneous mixture, the main lipid bilayer ingredients (e.g., phospholipid(s), cholesterol, etc.) are dissolved in an organic solvent (such as dichloromethane, chloroform, ethanol, or a chloroform-methanol mixture). Evaporation under vacuum pump at a temperature of 45-60° C. allows for the removal of the organic solvent. In certain embodiments, for small volumes (<1 mL), the organic solvent may be evaporated by means of a dry nitrogen or argon stream in a fume hood until the residual organic solvent is completely removed, while a rotary evaporation is usually used for larger volumes. After the removal of the organic solvent, a homogeneous, dry, thin-lipid film (of stacked bilayers) is formed. The lipid film is then hydrated using an appropriate aqueous medium (buffer) that, for pharmaceutical formulations, may consist, for example, of a solution of simple distilled water, or a normal (phosphate) saline buffer at, e.g., pH 7.4. The hydration process (typically with duration of 1 to 2 hrs) is generally performed at a temperature of 60-70° C., and in any case, above the phase-transition temperature of the bilayer component(s). To facilitate full lipid hydration, the final liposome suspension can be left overnight at a temperature of T˜4° C. During the hydration stage, the lipid becomes swollen and hydrated, resulting in the formation of a MLV suspension that is highly heterogeneous in size and lamellarity.

### Detergent Removal (Depletion) Method

In the detergent removal method, components of the lipid bilayer are hydrated (and solubilized) by using a detergent solution. Upon mixing, the detergent associates with the bilayer component(s) (shielding the hydrophobic portions from the direct interaction with the aqueous phase), and thus, mixed (detergent/lipid) micelles are formed. With the successive (progressive) removal of the detergent, the mixed micelles become richer in lipids and give rise to the formation of unilamellar vesicles. In various embodiments, commonly used detergents can include those with a high critical micelle concentration (CMC), such as sodium cholate, Triton X-100, sodium deoxycholate, alkyl glycoside, and the like. Detergent removal can be accomplished through various approaches.

The simplest method for detergent removal is dilution by, e.g., 10- to 100-fold by means of a buffer. Upon dilution with a buffer of the aqueous solution of a mixed lipid-detergent system, the size and polydispersity of the initial micelles increases. A spontaneous transition from polydispersed (elongated) micelles to vesicles occurs, as the system is diluted beyond the mixed micellar phase boundary. In the final stage of the detergent removal method, when the total detergent concentration becomes lower than the detergent's CMC, (proteo-) liposomes will form, and other methods are typically used to remove the residual detergent remaining in the nanoformulation. The detergent removal method has the main drawbacks of a final low concentration of liposomes, and a low entrapment efficiency of hydrophobic compounds.

An alternative to the detergent removal method is the detergent dialysis method, which furnishes an excellent reproducibility, with the final formation of homogenous size populations of liposomes. However, with this approach, traces of detergent(s) can be present within the liposomal nanoformulation. However, column gel chromatography, centrifugation, and the adsorption onto hydrophobic resin beads have been used as alternative efficient approaches for detergent removal.

The detergent removal technique permits the vesicles' formation with no degradation of their relevant biological activity and represents one of most employed methods for the reconstitution of (poorly soluble) membrane proteins. Main advantages of the detergent removal method include but are not limited to good control over the particle dimension and the product homogeneity, which strongly depend on the detergent removal rate and the initial detergent/phospholipid ratio. Some potential disadvantages of this method are connected with the slow equilibration process of the intermediate micellar aggregates, the presence of detergent residues, and the difficulty of removing the organic solvent.

### Solvent Injection Method

The solvent injection methods involve dissolution of the lipid bilayer component(s) into an organic solvent, and injection of the solution into an aqueous phase. Two main solvents (ethanol and ether) have typically been employed for the preparation of liposomal nanoformulation.

### Ethanol Injection.

In ethanol injection, the lipid bilayer component(s) (dissolved in ethanol) are injected into a pre-heated distilled water or TRIS-HCl buffer. The dilution of ethanol in the water solution below a critical concentration favors the self-assembly of the dissolved lipids in the aqueous phase. The rapid ethanol dilution (in the aqueous phase) also favors the lipid molecules' precipitation and the successive formation of bilayer planar fragments (stacks), which encapsulate the aqueous phase. Finally, the ethanol depletion (evaporation) favors the fusion of the lipids' fragments and the successive formation of closed unilamellar vesicles.

The volume of added ethanol represents an important factor in the formation of the liposomes. Typically, if the ethanol does not exceed 7.5% of the whole formulation volume, homogenous SUVs are formed. Conversely, if ethanol is rapidly injected (into a large excess of buffer) a heterogeneous population of MLVs are typically formed. The residual ethanol can be separated by a dialysis membrane, while the use of a filtration tube (under the pressure of, e.g., nitrogen gas) allows for concentration of the sample. With this approach, both LUV and SUV liposomes can be spontaneously formed. The ethanol can be removed by, for example, using a rotary evaporator (under nitrogen gas at reduced pressure, and T˜40° C.).

An automated high-throughput version of the ethanol injection method can be employed which uses a dedicated pipetting robot (for measuring and mixing volumes, mixing reservoir) in connection with a dynamic light scattering plate reader to characterize the liposomes in terms of size/distribution. This automated version favors the optimization of the amount of used materials, decreases the liposomes' production time (and costs), and facilitates the screening of many liposome properties in a shortened time.

Advantages of the ethanol injection technique include, but are not limited to the simplicity, the high level of reproducibility, the use of a non-harmful solvent such as ethanol, as well as the easy scale-up of the method. The main drawbacks are connected with the difficulty of removing the residual ethanol (as it forms azeotrope with water), and the final formation of a (very diluted) heterogeneous (30-110 nm) population of liposomes. Finally, there can be a risk of an inactivation of (biologically active) macromolecules in the presence of (even low amounts of) ethanol.

### Ether Injection

In ether injection, lipid bilayer component(s) dissolved in ether (or diethyl ether/methanol mixture), are (typically slowly) injected to an aqueous phase containing the components to be encapsulated, which are heated to a temperature range of typically ˜55° C.-65° C. in order to facilitate evaporation of the solvent from the liposomal product. Removal of the organic solvent (e.g., under reduced pressure) favors the generation of LUVs. Injection of an ether solution of lipid bilayer component(s) into the water phase causes the formation of SUVs from the evaporation of the ether solvent (the so-called ether vaporization method). An advantage of this approach (compared to the ethanol injection method) consists in the more efficient removal of the organic solvent from the final product. This favors the formation of concentrated liposome solutions with high entrapment efficiencies. The main limits of this method are the high polydispersity of the final population of liposomes (60 to 200 nm) and the fact that the active (or therapeutic) agents may be exposed to organic solvents and high temperatures.

### Reverse-Phase Evaporation

In a reverse-phase evaporation approach, lipid bilayer component(s) are dissolved in an organic solvent (e.g., a mixture of diethyl ether and chloroform (1:1 v/v), or diethyl ether/isopropyl ether, or chloroform/methanol (2:1 v/v)) which favors the formation of inverted micelles. A given quantity of an aqueous phase (e.g., a buffer) is added to the solution. The bilayer component(s), e.g., lipids, rearrange themselves at the interface between water and oil, creating a water-in-oil (W/O) microemulsion. The W/O microemulsion can be emulsified, by mechanical or sonication methods, to facilitate the formation of a homogeneous dispersion. With the aim of improving the liposomes formation efficiency, a phosphate saline (or citric-Na₂HPO₄) buffer can be added to aqueous phase. The use of a continued rotary evaporation (under reduced pressure) allows for the removal of the organic solvent, until the formation of a viscous gel. The slow organic solvent elimination favors the disruption of the inverted micelles and promotes the subsequent formation of liposomes (e.g., LUVs). At a given critical point, the gel collapses, while the excess of lipids in the solution environment distribute themselves around the inverted micelles to form a lipid bilayer around the (residual) water droplets, which results in the formation of the liposomes.

The large amount of the aqueous phase encapsulated by the microemulsions favors the encapsulation of a large amount of macromolecules within the liposomes. With this method, it is possible to encapsulate 30-45% of the aqueous volume, while (at optimal conditions) up to 65% of entrapment may be obtained. One drawback of this approach is connected with the presence of residual solvent (which can be removed by means of the dialysis and centrifugation methods) and with the difficulties in scaling-up the process.

### Microfluidic (Channel) Methods

In microfluidic methods of liposome formation, lipid bilayer component(s) dissolved for example in ethanol (or isopropanol) solvent, are successively propelled within microscopic channels (e.g., with ˜5-500-μm cross-section). The lipid bilayer component solution is focused between two aqueous streams in a microfluidic channel (microchannel), which generates a hydrodynamic laminar flow and diffusive mixing at the interface of the two liquids that favors lipids self-assembly into vesicles. With the precise control of mixing and the fluid flow rates, this method allows for the production of small (monodisperse) liposome nanoformulations with controllable sizes and distributions, with the use of low-toxicity solvents (such as ethanol). Compared to traditional bulk methods, the final product does not require post-production processing (i.e., extrusion, sonication).

A variety of new microfluidic methods have been developed for the formation of liposome nanoformulations for biomedical applications. Among many variations of this method, the micro hydrodynamic focusing (MHF) method developed by Jahn et al. (2007) Langmuir. 23: 6289-6293 is able to produce 40-140 nm homogeneous SUVs and LUVs with excellent control of the flow and mixing conditions. Moreover, high-throughput novel microfluidic architectures can be engineered for the mass production of liposome nanoformulation.

Modifications of the microfluidic technique include, but are not limited to continuous flow liposome formation based on the transmembrane pH (or ion) concentration gradient, which is created by using an on-chip microdialysis membrane (see, e.g., Bruna et al. (2022) Pharmaceutics, 14: 141; 135. Zhang et al. (2021) J. Nanomed. 16: 7391-7416; and the like). By using a thermoplastic microfabrication method, it has been possible to develop fully integrated microfluidic devices that favors a low-cost (scale-up) technology for the production of liposomal nanocarriers, in a (continuous) flow process. This integrated method (called pharmacy-on-a-chip) allows for the large-scale production (at about 100 mg/h lipid) of a new generation of fully optimized, multi-agent, and targeted liposomal nanoformulations (Id.).

The foregoing methods of liposome formation are illustrative and non-limiting. Using the teaching provided herein numerous methods of forming the liposomes described herein will be available to one of skill in the art.

### Downsizing and Post-Formation Processing.

For biomedical applications, the precise control of particle size (and polydispersity index-PDI), lamellarity, and homogeneity, can be an important step in liposome manufacturing and a fundamental parameter in the product specifications. In certain embodiments a post-formation processing can employed with the aim of breaking down initial large MLVs to obtain a final product. Sonication, extrusion, and the high-pressure homogenization methods, represent the most employed post-formation treatments for size reduction (downsizing).

### Sonication

The sonication method typically involves the application of a high (ultrasonic)-energy input (cavitation) to the MLVs liposome solution under a passive (inert) atmosphere. Two types of sonication techniques are typically used on an aqueous dispersion of a lipid bilayer component system—the bath sonication and probe sonication techniques. In the probe sonication method (generally used for small volumes), a sonicator tip is immersed into the liposome solution. The bath vessel is immersed into a water/ice bath to avoid high energy delivered by the tip, which causes a local warming-up and degradation of the lipidic solution.

### Extrusion Method

The extrusion method typically involve extrusion through pore-containing membranes (with sizes ranging from, e.g., 1 mm down to 25 nm). A heating block set around the extruder can allow for extrusion above the phase-transition temperature of the lipid bilaye component(s). Several passes through the membrane filters allow for the formation of (narrow-size distribution) LUV liposomes with dimensions close to the membrane pores' sizes. This method allows for a reproducible result of the final liposome product. A variation of this method is given by the maximator device, an extrusion setup consisting of a thermo-stable supply vessel connected to a high-pressure pumping system (see, e.g., Schneider et al. (1995) Int. J. Pharm. 117: 1-12).

Extrusion methods shown high reproducibility of downsizing. The main disadvantage of this approach is product losses, which can represent a limit for large-scale production. A different extrusion process for the production of liposome nanoformulations, called the French press method, is based on the extrusion of suspensions of MLVs through a small orifice, which results in the formation of SUVs (Hamilton et al. (1080) J. Lipid. Res. 21: 981-992). Liposomes formed with this setup are typically larger than those obtained by means of the sonication of MLVs.

### High-Pressure Homogenization

In high pressure homogenization, the initial liposome suspension (e.g., composed of multilamellar liposomes) is continuously injected through an orifice at a high pressure and collides with a fixed stainless-steel wall that causes downsizing of the liposomes (Brandl et al. (1990) Ind. Pharm. 16: 2167-2191). Liposome structure formation takes place due to cavitation, shear, and turbulence. With this method, the liposome size distribution may still be broad and variable. More specifically, the properties and the size distribution of the liposomes depends on the pressure, temperature, and the number of times that the lipidic system is processed within the homogenizer setup. A key role is also played by the initial properties (and factors) associated with the processed sample, including the lipids' (and bulk medium's) composition and ionic strength, and the initial liposomes' size-distribution and lamellarity.

### Post-Formation Processing of Liposomes

### Purification of Liposome Nanoformulations

Irrespective of the adopted formation method, non-encapsulated compounds (such as non-entrapped drugs, small molecules, or contaminant molecules) can be present in the external (liquid) environment of the generated liposomes and is desirably removed from the final nanoformulation through a purification process. Typical techniques employed for the removal of non-encapsulated materials include ultra-filtration, ultra-centrifugation, dialysis, and (size exclusion, gel-permeation, or ion-exchange) chromatography.

Another very important step of the post formation process is connected with the removal of residual organic solvents from the final liposomes. The use of the organic solvents (such as ethanol, methanol, chloroform, ether, and methylene chloride) can represent an important stage in the liposomes formation processes as it facilitates the molecular dispersion process of lipids and prevents the oxidation during the storage of the liposome component(s). However, residual solvents which are present in the final products may destabilize the liposomes. Although the (organic) solvents are usually removed by evaporation techniques, this process can cause a concentration of the lipids (and of unwanted the contaminants) in the residual solvents that is not easy to further remove.

Typically, liposome nanoformulations should be protected from oxidation. In various embodiments lipid nanocarriers can contain unsaturated lipids (acyl chains). During preparation, storage, or normal use, unsaturated lipids may undergo oxidative degradation (lipid peroxidation), a chemical process that involves some free radical reactions with the formation of cyclic-peroxides and hydro-peroxides. The lipid peroxidation process may be minimized by protecting them by keeping them under inert gases such as nitrogen or argon (in order to have minimal exposure to oxygen). Peroxidation can be minimized also by keeping liposome formulations in light-resistant containers or by the removal of heavy metals (e.g., by adding a chelating agent such as ethylenedinitrilotetraacetic—EDTA). The addition of antioxidants such as alpha-tocopherol or butylated hydroxytoluene can also minimize lipid oxidation processes.

### Sterilization of Liposomes

As parenteral routes are the most common routes of administration, liposome nanoformulations should desirably be free of viable microorganisms (such as bacteria, fungi, spores, etc.). For this reason, it is important to remove all possible microorganisms through a sterilization process. Sterilization can be achieved through various approaches, including, but not limited to, steam heating (autoclaving), ultraviolet and gamma ionizing irradiation, chemicals, and filtration methods.

Steam (autoclaving) sterilization consists of the combination of saturated steam under heat and pressure that causes the microorganism destruction by hydrolysis of proteins. However, many investigations have evidenced that this method is responsible for several liposome alterations that involve the oxidation and hydrolysis of lipids, phase transition and aggregation, and degradation (or leakage) of the encapsulated drugs. For this reason, this technique is suitable only for a limited number of liposome nanoformulations.

Heat sterilization may cause structural phase transitions (and correlated degradation and/or drug leakages), as well as the oxidation or hydrolysis of the component phospholipids (at higher temperatures (e.g., T>121° C.)). For this reason, this method is not generally used for liposome sterilization.

Although the gamma ionizing irradiation has a high-energy ionizing power (and then a strong penetration capacity), sterilization using gamma radiation may cause liposome degradation by lipid peroxidation (of unsaturated lipids), hydrolysis or fragmentation of component lipids, and changes in pH. Unlike gamma rays, ultraviolet (UV) radiation is a non-ionizing (low-energy) radiation with poor penetration capacity in materials, and for this reason, is not generally effective to cause the sensitive sterility of liposomes.

Ethylene oxide (chemical) sterilization uses ethylene oxide gas as a sterilizing antimicrobial agent whose sterilizing mechanism consists of the alkylation of the side chains of DNA, RNA, and enzymes, thus causing a strong metabolism inhibition and avoiding the multiplication of microorganisms. However, due to potential carcinogenicity/mutagenicity, the use of ethylene oxide is typically limited.

Sterilization by filtration is a relatively time-consuming method, based on the use of a sterile, disposable filtration unit consisting of an aseptic bacterial-free membrane (e.g., 0.22-μm) or depth filters for the removal of the microorganisms present in gaseous or liquid products.

Aseptic manufacturing consists in the preparation and the filling of a product in a controlled sterile environment (class A environment) equipped with sterile materials and equipment.

In view of the drawbacks of all these conventional approaches for liposome sterilization, alternative methods that ensure the sterility of liposomes in a green, effective, and inexpensive fashion are desirable. In this respect, one alternative method for liposome sterilization can be connected with the use of the supercritical carbon dioxide (SC—CO₂) technology (see, e.g., Soares et al. (2019) Mater. Sci. Eng. C. 99: 520-540). This method allows for producing and sterilizing liposome nanoformulations in a single step.

### Fabrication of Silicasomes.

As noted above, in certain embodiments the drug delivery vehicles described herein are “silicasomes” that comprise lipid-bilayer coated nanoparticles (e.g., mesoporous silica nanoparticles). Methods of making silicasomes are known to those of skill in the art and described, for example in PCT Application Numbers: PCT/US2020/055585 (WO 2021/076630), PCT/US2018/067970 (WO 2019/133884), PCT/US2017/012625 (WO 2017/120537), and the like.

The methods of silicasome fabrication typically involve synthesis of a mesoporous nanoparticle (e.g., a mesoporous silica nanoparticle (MSNP). Loading of drug(s) can be accomplished by a remote loading technique that involves encapsulating a protonating agent in the pores of the nanoparticle which subsequently allows the drug (e.g., CXCR4 antagonist) to be imported across the LB by a proton gradient.

The mesoporous nanoparticle is then coated with a lipid bilayer as described herein.

### Fabrication of Mesoporous Silica Cores.

MSNP cores can be synthesized using a laboratory protocol described, inter alia, by Liu et al. (2016) ACS Nano, 10(2): 2702-2715. Generally, MSNP cores are synthesized by a sol/gel procedure. For example, in one illustrative, but non-limiting embodiment 50 mL of CTAC (cetyltrimethylammonium chloride) is mixed with 150 mL of H₂O in a 500 mL conical flask, followed by stirring at 350 rpm for 15 min at 85° C. This is followed by the addition of 8 mL of 10% triethanolamine (TEA) for 30 min at the same temperature. Then, 7.5 mL of the silica precursor, tetraethyl orthosilicate (TEOS), is added dropwise at a rate of 1 mL/min using a peristaltic pump. The solution is stirred at 350 rpm at 85° C. for 20 min, leading to the formation particles with a primary size of ˜65 nm. The surfactant is removed by washing the particles with a mixture of methanol/HCl (500:19 v/v) at room temperature for 24 h. The particles were centrifuged at 10,000 rpm for 60 min and washed three times in methanol.

While, as described above, in various embodiments, the surfactant CTAC is used, in certain other embodiments any of a number of surfactants including, but not limited to anionic surfactants or cationic surfactants can be used. Illustrative, but non-limiting examples of anionic surfactants include a dodecylsulfate salt (e.g., sodium dodecylsulfate or lithium dodecylsulfate (SDS)), and illustrative, but non-limiting examples of cationic surfactants include, but are not limited to, a tetradecyl-trimethyl-ammonium salt (e.g., tetradecyl-trimethyl-ammonium bromide (C14TAB; CTAB) or tetradecyl-trimethyl-ammonium chloride (CTAC), a hexadecyltrimethylammonium salt (e.g., hexadecyl-trimethylammonium bromide (C16; CTAB)), an octadecyltrimethylammonium salt (e.g., octadecyltrimethylammonium bromide (C18; OTAB)), a dodecylethyldimethylammonium salt (e.g., dodecylethyldimethylammonium bromide), a cetylpyridinium salt (e.g., cetylpyridinium chloride (CPC)), polyethoxylated tallow amine (POEA), hexadecyl trimethylammonium p-toluenesulfonate, a benzalkonium salt (e.g., benzalkonium chloride (BAC)), or a benzethonium salt (e.g., benzethonium chloride (BZT)) and mixtures thereof. In certain embodiments the use of cationic surfactants (e.g., CTAC) is preferred.

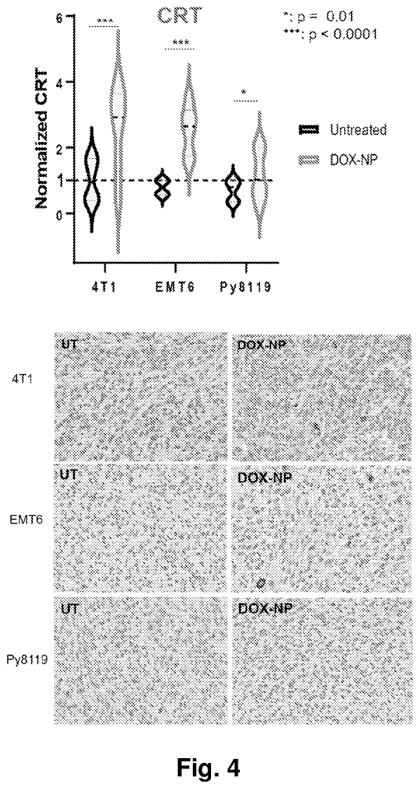

Determination of desirable conditions for large-scale synthesis required the full elucidation of the sol-gel reaction, followed by fine-tuning and iterative condition testing as described in PCT Application No: PCT/US2018/067970. As described therein, synthesis conditions depicted in batch 49 (see, e.g., PCT/US2018/067970 FIG. 4, panels B, C) provided an integrated set of synthesis parameters that permit effective scale-up synthesis of therapeutic MSNPs. The synthesis conditions shown in batch 71 described therein (see, e.g., PCT/US2018/067970 FIG. 4, panels D, and E) provided other representative optimal conditions in a 20 L reaction system. It is believed these parameters (with some variation) are effective for large scale synthesis of a substantially homogenous population of MSNPs having the desired size and porosity.

Accordingly, in certain embodiments, methods for the large-scale (e.g., about 20 g, or greater, or about 30 g or greater, or about 40 g or greater, or about 50 g or greater, or about 60 g or greater, or about 80 g or greater, or about 1 kg or greater in a single batch) preparation of mesoporous silica nanoparticles suitable use in pharmaceuticals are provided where the methods involve providing cetyltrimethylammonium chloride (CTAC) in water at a concentration greater than the CTAC critical micellar concentration (CMC) to form a mixture comprising CTAC micelles; adding to the mixture triethanolamine (TEA); adding to the mixture tetraethylorthosilicate (TEOS) where the molar ratio of H₂O:CTAC:TEA:TEOS ranges from about 100 to about 150 water:about 0.06 to about 0.10 CTAC:about 0.04 to about 0.08 TEA:about 0.8 to about 1.2 TEOS; and stirring (or agitating) the mixture to allow the CTAC micelles, TEA, and TEOS to react to form a population of mesoporous silica nanoparticles (MSNPs). In certain embodiments the method produces at least 20 g or greater, or 40 g or greater, or 50 g or greater, or 60 g or greater, or 80 g or greater, or 1 kg or greater MSNPs in a single batch. In certain embodiments the ratio of H₂O:CTAC:TEA:TEOS molar ratio is about 125:0.08:0.06:1 and, in certain embodiments, ranges from about 100 to about 150 water:about 0.06 to about 0.10 CTAC:about 0.04 to about 0.08 TEA:about 0.8 to about 1.2 TEOS. In certain embodiments the method comprises combining about 3,000 mL water, about 36.3 g CTAC, about 12 g TEA and about 280 g TEOS.

In some embodiments, the molar ratio of H₂O (water):CTAC:TEA:TEOS is about 100 to about 110 water, about 110 to about 120 water, about 120 to about 130 water, about 130 to about 140 water, about 140 to about 150 water, about 100 to about 120 water, about 110 to about 130 water, about 120 to about 140 water, or about 130 to about 150 water:about 0.06 to about 0.07 CTAC, about 0.07 to about 0.08 CTAC, about 0.08 to about 0.09 CTAC, about 0.09 to about 0.10 CTAC, about 0.06 to about 0.08 CTAC, about 0.07 to about 0.09 CTAC, or about 0.08 to about 0.10 CTAC:about 0.04 to about 0.05 TEA, about 0.05 to about 0.06 TEA, about 0.06 to about 0.07 TEA, about 0.07 to about 0.08 TEA, about 0.04 to about 0.06 TEA, about 0.05 to about 0.07 TEA, or about 0.06 to about 0.08 TEA:about 0.8 to about 0.9 TEOS, about 0.9 to about 1.0 TEOS, about 1.0 to about 1.1 TEOS, about 1.1 to about 1.2 TEOS, about 0.8 to about 1.0 TEOS, about 0.9 to about 1.1 TEOS, or about 1.0 to about 1.2 TEOS. In addition to CTAC, these molar ratios are also contemplated for other surfactants described herein such as, for example, C14TAB, CTAB, OTAB, CPC, POEA, BAC, BZT, other suitable surfactants, and mixtures thereof.

In certain embodiments the method is performed at a temperature ranging from about 75° C. to about 90° C. (e.g., at about 85° C.). In some embodiments, the method is performed at a reaction temperature of about 70° C. to about 95° C. In some embodiments, the method is performed at a reaction temperature of about 70° C. to about 75° C., about 70° C. to about 80° C., about 70° C. to about 85° C., about 70° C. to about 90° C., about 70° C. to about 95° C., about 75° C. to about 80° C., about 75° C. to about 85° C., about 75° C. to about 90° C., about 75° C. to about 95° C., about 80° C. to about 85° C., about 80° C. to about 90° C., about 80° C. to about 95° C., about 85° C. to about 90° C., about 85° C. to about 95° C., or about 90° C. to about 95° C. In some embodiments, the method is performed at a reaction temperature of about 70° C., about 75° C., about 80° C., about 85° C., about 90° C., or about 95° C. In some embodiments, the method is performed at a reaction temperature of at least about 70° C., about 75° C., about 80° C., about 85° C., or about 90° C. In some embodiments, the method is performed at a reaction temperature of at most about 75° C., about 80° C., about 85° C., about 90° C., or about 95° C. In some embodiments, the method is performed at a reaction volume of about 1 L to about 50 L. In some embodiments, the method is performed at a reaction volume of about 1 L to about 5 L, about 1 L to about 10 L, about 1 L to about 15 L, about 1 L to about 18 L, about 1 L to about 20 L, about 1 L to about 25 L, about 1 L to about 30 L, about 1 L to about 40 L, about 1 L to about 50 L, about 5 L to about 10 L, about 5 L to about 15 L, about 5 L to about 18 L, about 5 L to about 20 L, about 5 L to about 25 L, about 5 L to about 30 L, about 5 L to about 40 L, about 5 L to about 50 L, about 10 L to about 15 L, about 10 L to about 18 L, about 10 L to about 20 L, about 10 L to about 25 L, about 10 L to about 30 L, about 10 L to about 40 L, about 10 L to about 50 L, about 15 L to about 18 L, about 15 L to about 20 L, about 15 L to about 25 L, about 15 L to about 30 L, about 15 L to about 40 L, about 15 L to about 50 L, about 18 L to about 20 L, about 18 L to about 25 L, about 18 L to about 30 L, about 18 L to about 40 L, about 18 L to about 50 L, about 20 L to about 25 L, about 20 L to about 30 L, about 20 L to about 40 L, about 20 L to about 50 L, about 25 L to about 30 L, about 25 L to about 40 L, about 25 L to about 50 L, about 30 L to about 40 L, about 30 L to about 50 L, or about 40 L to about 50 L. In some embodiments, the method is performed at a reaction volume of about 1 L, about 5 L, about 10 L, about 15 L, about 18 L, about 20 L, about 25 L, about 30 L, about 40 L, or about 50 L. In some embodiments, the method is performed at a reaction volume of at least about 1 L, about 5 L, about 10 L, about 15 L, about 18 L, about 20 L, about 25 L, about 30 L, or about 40 L. In some embodiments, the method is performed at a reaction volume of at most about 5 L, about 10 L, about 15 L, about 18 L, about 20 L, about 25 L, about 30 L, about 40 L, or about 50 L.

In certain embodiments, e.g., to make about 60 g to about 70 g bare MSNP, the molar ratio of H₂O:CTAC:TEA:TEOS is about 125:0.08:0.06:1 and the temperature is at about 85° C. for about 2 hrs, in a reaction volume of about 3 L. After reaction, the system can be naturally cooled to room temperature. While CTAC is used in various embodiments, other surfactants are also contemplated such as, for example, C14TAB, CTAB, OTAB, CPC, POEA, BAC, BZT, other suitable surfactants, and mixtures thereof.

In certain embodiments, e.g., to make about 120 g to about 140 g bare MSNP, the molar ratio of H₂O:CTAC:TEA:TEOS is about 125:0.08:0.06:0.33, and the temperature is at about 85° C. for about 4 hrs in a reaction volume of about 18 L. After reaction, the system can be naturally cooled to room temperature.

In certain embodiments the stirring or agitating comprises stirring at a speed ranging from about 150 rpm, or from about 200 rpm, or from about 250 rpm up to about 800 rpm, or up to about 600 rpm, or up to about 400 rpm, or up to about 300 rpm. In certain embodiments the stirring or agitating comprises stirring at about 250 rpm. In certain embodiments the reaction proceeds until the hydrodynamic size of the MSNPs is substantially constant and/or where the yield of MSNPs is substantially constant.

In certain embodiments the stirring or agitating comprises stirring at a speed of about 150 rpm to about 800 rpm. In certain embodiments the stirring or agitating comprises stirring at a speed of about 150 rpm to about 200 rpm, about 150 rpm to about 300 rpm, about 150 rpm to about 400 rpm, about 150 rpm to about 500 rpm, about 150 rpm to about 600 rpm, about 150 rpm to about 700 rpm, about 150 rpm to about 800 rpm, about 200 rpm to about 300 rpm, about 200 rpm to about 400 rpm, about 200 rpm to about 500 rpm, about 200 rpm to about 600 rpm, about 200 rpm to about 700 rpm, about 200 rpm to about 800 rpm, about 300 rpm to about 400 rpm, about 300 rpm to about 500 rpm, about 300 rpm to about 600 rpm, about 300 rpm to about 700 rpm, about 300 rpm to about 800 rpm, about 400 rpm to about 500 rpm, about 400 rpm to about 600 rpm, about 400 rpm to about 700 rpm, about 400 rpm to about 800 rpm, about 500 rpm to about 600 rpm, about 500 rpm to about 700 rpm, about 500 rpm to about 800 rpm, about 600 rpm to about 700 rpm, about 600 rpm to about 800 rpm, or about 700 rpm to about 800 rpm. In certain embodiments the stirring or agitating comprises stirring at a speed of about 150 rpm, about 200 rpm, about 300 rpm, about 400 rpm, about 500 rpm, about 600 rpm, about 700 rpm, or about 800 rpm. In certain embodiments the stirring or agitating comprises stirring at a speed of at least about 150 rpm, about 200 rpm, about 300 rpm, about 400 rpm, about 500 rpm, about 600 rpm, or about 700 rpm. In certain embodiments the stirring or agitating comprises stirring at a speed of at most about 200 rpm, about 300 rpm, about 400 rpm, about 500 rpm, about 600 rpm, about 700 rpm, or about 800 rpm.

In certain embodiments the reaction proceeds for a time period of about 1.5 hours, and in certain embodiments, the time period ranges from about 0.5 hr, or from about 1 hour, up to about 5 hours or up to about 4 hours, or up to about 3 hours, or up to about 2 hours. In some embodiments, the reaction proceeds for a time period of about 0.5 hours to about 5 hours. In some embodiments, the reaction proceeds for a time period of about 0.5 hours to about 1 hour, about 0.5 hours to about 1.5 hours, about 0.5 hours to about 2 hours, about 0.5 hours to about 3 hours, about 0.5 hours to about 4 hours, about 0.5 hours to about 5 hours, about 1 hour to about 1.5 hours, about 1 hour to about 2 hours, about 1 hour to about 3 hours, about 1 hour to about 4 hours, about 1 hour to about 5 hours, about 1.5 hours to about 2 hours, about 1.5 hours to about 3 hours, about 1.5 hours to about 4 hours, about 1.5 hours to about 5 hours, about 2 hours to about 3 hours, about 2 hours to about 4 hours, about 2 hours to about 5 hours, about 3 hours to about 4 hours, about 3 hours to about 5 hours, or about 4 hours to about 5 hours. In some embodiments, the reaction proceeds for a time period of about 0.5 hours, about 1 hour, about 1.5 hours, about 2 hours, about 3 hours, about 4 hours, or about 5 hours. In some embodiments, the reaction proceeds for a time period of at least about 0.5 hours, about 1 hour, about 1.5 hours, about 2 hours, about 3 hours, or about 4 hours. In some embodiments, the reaction proceeds for a time period of at most about 1 hour, about 1.5 hours, about 2 hours, about 3 hours, about 4 hours, or about 5 hours.

In certain embodiments the method has a yield of greater than about 80%. In certain embodiments the method produces MSNPs having a substantially monotonic size distribution. In certain embodiments the method produces MSNPs whose size distribution has a coefficient of variation of less than about 0.10. In certain embodiments the method produces MSNPs having an average diameter ranging from about 60 nm up to about 70 nm (e.g., an average diameter of about 65-66 nm). In certain embodiments the method produces MSNPs having an average pore size ranging from about 2.2 to about 2.7 nm, or from about 2.3 to about 2.6 nm, or ranging from about 2.4-2.5 nm. In certain embodiments the synthesis is performed in a reaction vessel or in a microfluidic reactor.

In some embodiments, the method produces MSNPs whose size distribution has a coefficient of variation of about 0.01 to about 0.3. In some embodiments, the method produces MSNPs whose size distribution has a coefficient of variation of about 0.01 to about 0.05, about 0.01 to about 0.1, about 0.01 to about 0.15, about 0.01 to about 0.2, about 0.01 to about 0.25, about 0.01 to about 0.3, about 0.05 to about 0.1, about 0.05 to about 0.15, about 0.05 to about 0.2, about 0.05 to about 0.25, about 0.05 to about 0.3, about 0.1 to about 0.15, about 0.1 to about 0.2, about 0.1 to about 0.25, about 0.1 to about 0.3, about 0.15 to about 0.2, about 0.15 to about 0.25, about 0.15 to about 0.3, about 0.2 to about 0.25, about 0.2 to about 0.3, or about 0.25 to about 0.3. In some embodiments, the method produces MSNPs whose size distribution has a coefficient of variation of about 0.01, about 0.05, about 0.1, about 0.15, about 0.2, about 0.25, or about 0.3. In some embodiments, the method produces MSNPs whose size distribution has a coefficient of variation of at least about 0.01, about 0.05, about 0.1, about 0.15, about 0.2, or about 0.25. In some embodiments, the method produces MSNPs whose size distribution has a coefficient of variation of at most about 0.05, about 0.1, about 0.15, about 0.2, about 0.25, or about 0.3.

In some embodiments, the method produces MSNPs having an average diameter of about 30 nm to about 300 nm. In some embodiments, the method produces MSNPs having an average diameter of about 30 nm to about 40 nm, about 30 nm to about 50 nm, about 30 nm to about 60 nm, about 30 nm to about 70 nm, about 30 nm to about 80 nm, about 30 nm to about 90 nm, about 30 nm to about 100 nm, about 30 nm to about 150 nm, about 30 nm to about 200 nm, about 30 nm to about 250 nm, about 30 nm to about 300 nm, about 40 nm to about 50 nm, about 40 nm to about 60 nm, about 40 nm to about 70 nm, about 40 nm to about 80 nm, about 40 nm to about 90 nm, about 40 nm to about 100 nm, about 40 nm to about 150 nm, about 40 nm to about 200 nm, about 40 nm to about 250 nm, about 40 nm to about 300 nm, about 50 nm to about 60 nm, about 50 nm to about 70 nm, about 50 nm to about 80 nm, about 50 nm to about 90 nm, about 50 nm to about 100 nm, about 50 nm to about 150 nm, about 50 nm to about 200 nm, about 50 nm to about 250 nm, about 50 nm to about 300 nm, about 60 nm to about 70 nm, about 60 nm to about 80 nm, about 60 nm to about 90 nm, about 60 nm to about 100 nm, about 60 nm to about 150 nm, about 60 nm to about 200 nm, about 60 nm to about 250 nm, about 60 nm to about 300 nm, about 70 nm to about 80 nm, about 70 nm to about 90 nm, about 70 nm to about 100 nm, about 70 nm to about 150 nm, about 70 nm to about 200 nm, about 70 nm to about 250 nm, about 70 nm to about 300 nm, about 80 nm to about 90 nm, about 80 nm to about 100 nm, about 80 nm to about 150 nm, about 80 nm to about 200 nm, about 80 nm to about 250 nm, about 80 nm to about 300 nm, about 90 nm to about 100 nm, about 90 nm to about 150 nm, about 90 nm to about 200 nm, about 90 nm to about 250 nm, about 90 nm to about 300 nm, about 100 nm to about 150 nm, about 100 nm to about 200 nm, about 100 nm to about 250 nm, about 100 nm to about 300 nm, about 150 nm to about 200 nm, about 150 nm to about 250 nm, about 150 nm to about 300 nm, about 200 nm to about 250 nm, about 200 nm to about 300 nm, or about 250 nm to about 300 nm. In some embodiments, the method produces MSNPs having an average diameter of about 30 nm, about 40 nm, about 50 nm, about 60 nm, about 70 nm, about 80 nm, about 90 nm, about 100 nm, about 150 nm, about 200 nm, about 250 nm, or about 300 nm. In some embodiments, the method produces MSNPs having an average diameter of at least about 30 nm, about 40 nm, about 50 nm, about 60 nm, about 70 nm, about 80 nm, about 90 nm, about 100 nm, about 150 nm, about 200 nm, or about 250 nm. In some embodiments, the method produces MSNPs having an average diameter of at most about 40 nm, about 50 nm, about 60 nm, about 70 nm, about 80 nm, about 90 nm, about 100 nm, about 150 nm, about 200 nm, about 250 nm, or about 300 nm.

In some embodiments, the method produces MSNPs having an average pore size of about 2 nm to about 4 nm. In some embodiments, the method produces MSNPs having an average pore size of about 2 nm to about 2.2 nm, about 2 nm to about 2.4 nm, about 2 nm to about 2.6 nm, about 2 nm to about 2.8 nm, about 2 nm to about 3 nm, about 2 nm to about 3.2 nm, about 2 nm to about 3.4 nm, about 2 nm to about 3.6 nm, about 2 nm to about 3.8 nm, about 2 nm to about 4 nm, about 2.2 nm to about 2.4 nm, about 2.2 nm to about 2.6 nm, about 2.2 nm to about 2.8 nm, about 2.2 nm to about 3 nm, about 2.2 nm to about 3.2 nm, about 2.2 nm to about 3.4 nm, about 2.2 nm to about 3.6 nm, about 2.2 nm to about 3.8 nm, about 2.2 nm to about 4 nm, about 2.4 nm to about 2.6 nm, about 2.4 nm to about 2.8 nm, about 2.4 nm to about 3 nm, about 2.4 nm to about 3.2 nm, about 2.4 nm to about 3.4 nm, about 2.4 nm to about 3.6 nm, about 2.4 nm to about 3.8 nm, about 2.4 nm to about 4 nm, about 2.6 nm to about 2.8 nm, about 2.6 nm to about 3 nm, about 2.6 nm to about 3.2 nm, about 2.6 nm to about 3.4 nm, about 2.6 nm to about 3.6 nm, about 2.6 nm to about 3.8 nm, about 2.6 nm to about 4 nm, about 2.8 nm to about 3 nm, about 2.8 nm to about 3.2 nm, about 2.8 nm to about 3.4 nm, about 2.8 nm to about 3.6 nm, about 2.8 nm to about 3.8 nm, about 2.8 nm to about 4 nm, about 3 nm to about 3.2 nm, about 3 nm to about 3.4 nm, about 3 nm to about 3.6 nm, about 3 nm to about 3.8 nm, about 3 nm to about 4 nm, about 3.2 nm to about 3.4 nm, about 3.2 nm to about 3.6 nm, about 3.2 nm to about 3.8 nm, about 3.2 nm to about 4 nm, about 3.4 nm to about 3.6 nm, about 3.4 nm to about 3.8 nm, about 3.4 nm to about 4 nm, about 3.6 nm to about 3.8 nm, about 3.6 nm to about 4 nm, or about 3.8 nm to about 4 nm. In some embodiments, the method produces MSNPs having an average pore size of about 2 nm, about 2.2 nm, about 2.4 nm, about 2.6 nm, about 2.8 nm, about 3 nm, about 3.2 nm, about 3.4 nm, about 3.6 nm, about 3.8 nm, or about 4 nm. In some embodiments, the method produces MSNPs having an average pore size of at least about 2 nm, about 2.2 nm, about 2.4 nm, about 2.6 nm, about 2.8 nm, about 3 nm, about 3.2 nm, about 3.4 nm, about 3.6 nm, or about 3.8 nm. In some embodiments, the method produces MSNPs having an average pore size of at most about 2.2 nm, about 2.4 nm, about 2.6 nm, about 2.8 nm, about 3 nm, about 3.2 nm, about 3.4 nm, about 3.6 nm, about 3.8 nm, or about 4 nm.

In certain embodiments the method comprises removing the CTAC surfactant by a wash procedure (e.g., washing the MSNPs with an alcohol and/or an acid). In certain embodiments the wash procedure comprises washing the MSNPs with an alcohol/acid mixture. In certain embodiments the alcohol/acid mixture comprises a methanol/HCl mixture (e.g., methanol/HCL at 500:19 v/v) and the washing is, optionally, at room temperature. In certain embodiments the method further comprises centrifuging and/or washing the MSNPs.

### Lipid Bilayer (LB) Formation on the MSNPs

To make small (e.g., a few hundred mg) batches of silicasomes, we used a one-step biofilm encapsulation method which holds significant advances over other methods of MSNP bilayer coating, such as the liposome fusion approach (see, e.g., Brinker J et al. (2009) J. Am. Chem. Soc., 131: 7567-7569). The biofilm approach involved creation of a biofilm and then application of MSNPs to the film and sonication. Without being bound to a particular theory, it is believed that, using this method, van der Waals forces contribute to the rapid and complete coating of the MSNP surface. Our published protocol (Liu et al. (2016) ACS Nano, 10(2): 2702-2715) showed that 220 mg lipid mixture can make biofilm at ˜75 cm² with a thickness of about 24 pm in a 150 mL flask, which is enough to coat about 200 mg MSNPs.

However, it was determined that the lipid-bilayer approach is unsuitable for scale-up to large scale silicasome synthesis. Based on LB surface area, it is possible to calculate the rehydration reaction volume for different silicasome batch sizes). For example, to make 100 g/batch silicasome for, e.g., a phase 2 study, we need to generate about 37,500 cm² biofilm, which requires about a 1,900 L rehydration reactor. This is impractical because of the huge reaction volume, difficulty of particle quality control, significant sample purification burden and cost concerns. Accordingly, we sought to develop alternative methodologies for MSNP pore sealing (LB formation on MSNPs) with a view to, inter alia, reducing reaction volume. In other words, we sought to provide uniform and intact MSNP pore sealing (LB formation on MSNPs) in a concentrated system.

In order to reduce the reaction volume, our initial attempt was to make thicker LB. The resulting biofilm was non-uniform. The coating was inefficient for MSNP pore sealing, and essentially downgraded the effective thin biofilm approach to the ineffective liposome fusion method.

In order to establish an effective method of lipid bilayer (LB) encapsulation of MSNPs (MSNP pore sealing) for large batches, we developed a novel a novel solvent precipitation method (e.g., a “lipid ethanol solution” method). Unlike previous lipid coating procedures (see, e.g., the liposome fusion method described in Brinker et al. (2009) Am. Chem. Soc. 131: 7567-7569, and the biofilm encapsulation methods described by Meng et al. (2015) ACS Nano, 9(4): 3540-3557), the “solvent precipitation” allows utilization of much more concentrated lipid or particle solutions/suspensions, which makes possible the large-scale synthesis of silicasomes.

In one embodiment of a new “solvent precipitation” instead of making lipid biofilm, we introduced trapping agent (e.g., protonating agent) soaked MSNP into a highly concentrated lipid(s) ethanol solution at appropriate temperature (e.g., 65° C.). In some embodiments, the appropriate temperature is about 55° C. to about 75° C. In some embodiments, the appropriate temperature is about 55° C. to about 60° C., about 55° C. to about 65° C., about 55° C. to about 70° C., about 55° C. to about 75° C., about 60° C. to about 65° C., about 60° C. to about 70° C., about 60° C. to about 75° C., about 65° C. to about 70° C., about 65° C. to about 75° C., or about 70° C. to about 75° C. In some embodiments, the appropriate temperature is about 55° C., about 60° C., about 65° C., about 70° C., or about 75° C. In some embodiments, the appropriate temperature is at least about 55° C., about 60° C., about 65° C., or about 70° C. In some embodiments, the appropriate temperature is at most about 60° C., about 65° C., about 70° C., or about 75° C.

While various parameters, including, but not limited to aqueous solution/ethanol volume ratio and sonication conditions, effective large scale synthesis was accomplished using the parameters for lipid concentration, temperature, MSNP:lipid ratio, etc. as described below. The illustrated protocol permitted the provision of large-scale (large batch) effective, uniform, and intact LB coating of MSNPs.

In the illustrated solvent precipitation approach which utilized a lipid ethanol solution, x mg MSNPs are soaked in a x/40 mL protonating (trapping agent) (e.g., TEA₈SOS 80 mM) solution, which is added to a mixture of lipids in x/400 mL ethanol at 65° C., comprised of a x:1.1 mg mixture of DSPC/Chol/DSPE-PEG2000 (molar ratio 3:2:0.15). This equals to a MSNP concentration of 40 mg/mL, MSNP:Lipid ratio of 1:1.1 w/w, and a lipid concentration of ˜440 mg/mL. In certain embodiments the alcohol (e.g., ethanol) is a 100% absolute alcohol (e.g., absolute ethanol), while in other embodiments the alcohol is a 97% alcohol, or in certain embodiments a 95% alcohol.

The mixture is then sonicated using a probe sonicator with a 15/15 s on/off working cycle and a power output of 52 W to obtain a clear suspension. Free TEA₈SOS can be removed by size exclusion chromatography over a Sepharose CL-4B column. In certain embodiments alternatives to a probe sonicator can be used. Such alternatives include but are not limited to a static sonicator (homogenizer), or a dynamic flow system (homogenizer/sonicator) with an energy input function, both of which provide energy control for effective lipid coating without unwanted damage that may lead to overheating or raw material degradation. One illustrative, but non-limiting example is the SONOLATOR® (Sonic Corp.).

In general, any device that provides substantial and controllable intensity of ultrasound and high ultrasonic vibration amplitudes. Such devices include but are not limited to “direct sonication” equipment, which usually refers to the ultrasound that is directly coupled into the processing liquid. Examples include but are not limited to probe-type ultrasonicators. The coating can also be achieved by the use of “indirect sonication” equipment, which means the coupling of the ultrasound waves via ultrasonic bath through a container's wall into the sample liquid, e.g. VialTweeter, CupHorn, and the like.

In one illustrative, but non-limiting embodiment a probe flow through sonicator is used because this is one of the most popular setups used in pharmaceutical preparation.

The optimal sonication conditions can be determined using routine methods. In one illustrative, but non-limiting embodiment, probe sonication is used to coat 20 g silicasome at a power of 200 W, using a 15 s/5 s on/off cycle for 2 hr. This can also be achieved using flow sonication system using continuous power input of 400 W at flow rate of 10 m/min. For the flow sonication, the total time for making 20 g silicasome is about ˜100 min.

The protonating (trapping agent) loaded silicasomes are incubated in a drug (e.g., CXCR4 antagonist) solution for drug loading, e.g., in a water bath at 65° C. The loading can be stopped after, e.g., 30 min by quenching in and ice water bath, following which the drug-loaded silicasomes are washed by centrifugation and re-suspended in PBS.

While the solvent-precipitation of lipid bilayer formation on MSNPs described above utilized an ethanol solution, a TEA₈SOS trapping agent (protonating agent), and particular composition lipids, in certain embodiments, other solvents can be used, other trapping agents can be used, and different lipid bilayer compositions can be utilized.

Accordingly, in certain embodiments, the solvent comprises a polar solvent selected from the group consisting of ethanol, methanol, or an ethanol or methanol containing aqueous solvent with the organic phase greater than 95% w/w. In certain embodiments the ratio of MSNP to lipid ranges from about 1:3 to about 1:1, or from about 1:2 to about 1:15, or from about 1:2 to about 1:1 (w/w), while as illustrated above, in certain embodiments the ratio of MSNP to lipid is about 1:1.1 (wt/wt). In some embodiments, the ratio of MSNP to lipid is at least about 1:1 (w/w), at least about 1:1.1, at least about 1:1.2, at least about 1:1.3, at least about 1:1.4, at least about 1:1.5, at least about 1:2, at least about 1:3, at least about 1:4, at least about 1:5, at least about 1:6, at least about 1:7, at least about 1:8, at least about 1:9, at least about 1:10, at least about 1:11, at least about 1:12, at least about 1:13, at least about 1:14, or at least about 1:15 or more. In some embodiments, the ratio of MSNP to lipid is no more than about 1:1 (w/w), no more than about 1:1.1, no more than about 1:1.2, no more than about 1:1.3, no more than about 1:1.4, no more than about 1:1.5, no more than about 1:2, no more than about 1:3, no more than about 1:4, no more than about 1:5, no more than about 1:6, no more than about 1:7, no more than about 1:8, no more than about 1:9, no more than about 1:10, no more than about 1:11, no more than about 1:12, no more than about 1:13, no more than about 1:14, or no more than about 1:15 or more.

In typical embodiments, the temperature is greater than the liquid transition temperature for each component. In certain embodiments the reaction is performed at a temperature ranging from about 40° C., or from about 50° C., or from about 60° C., to about 80° C., or to about 75° C., or to about 70° C. In certain embodiments the reaction is performed at a temperature of about 65° C. In certain embodiments the sonication proceeds at an energy and duration sufficient to provide a substantially clear suspension of silicasomes.

Various lipid formulations for the lipid bilayer, trapping agents, and silicasome features are described below.

Like with liposomes, silicasomes are also subject to post-formulation processing. Such processing can include purification and/or sterilization, e.g., as discussed above for liposome fabrication.

### Remote Loading of Silicasomes, and Liposomes

In certain embodiments, the encapsulation of, e.g., the CXCR4 antagonist (and/or other drugs) in the silicasome and/or in the liposome can be optimized by using a “remote loading” strategy in which the addition of the drug (e.g., the CXCR4 antagonist) to preformed vesicles or silicasomes (LB-coated nanoparticles) which achieves high loading levels using a pH gradient or an ion gradient capable of generating a pH gradient (see, e.g., Ogawa et al. (2009) J. Control. Rel. 1(5): 4-10; Fritze et al. (2006) Biochimica et Biophys Acta. 1758: 1633-1640). In general, the remote loading method involves adding a cargo-trapping reagent (e.g., a protonating reagent such as TEA₈SOS, ammonium sulfate, etc.) which can be added to the lipid biofilm prior to the sonication in the formation of silicasomes, or can be incorporated into the liposome lipids prior to the formation of the liposome.

Thus for example, a CXCR4 antagonist containing liposome can be prepared as follows: 1) a total of 50 mg lipids (e.g., DSPC/Chol/DSPE-PEG (e.g., DSPE-PEG2k, DSPE-PEG5k, and the like), in certain embodiments at a molar ratio of 3:2:0.15 can be dissolved in 5 mL chloroform in a 50 mL round bottom glass flask. The solvent can be evaporated under a rotatory vacuum to form a uniform thin lipid film that can be dried further under vacuum overnight. The film can be hydrated with a cargo-trapping agent (e.g., with 2 mL of ammonium sulfate (123 mM) and probe sonicated, e.g., for 1 h, then subsequently extruded, e.g., 15 times, through a Mini-Extruder (Avanti Polar Lipids), using, e.g., a polycarbonate membrane with 100 nm pores (Avanti Polar Lipids) at 80° C. IND nanovesicle (IND-NV) size and morphology can be assessed by dynamic light scattering and cryoEM, respectively as desired. Unincorporated cargo-trapping agent (e.g., ammonium sulfate) can be removed, e.g., by running through a PD-10 size exclusion column. The drug to be loaded (e.g., CXCR4 antagonist) in DI water) can be incubated with the above prepared liposomes, e.g., at 65° C. for 40 min. The nanovesicles can be fractionated across a PD-10 column, allowing the removal of free drug. Their size and morphology can be assessed by dynamic light scattering, cryoEM and UPLC/MS-MS, respectively. In other illustrative, but non-limiting embodiments, citrate or TEA8SOS can be used to load the drug(s).

Of course, this protocol is illustrative and non-limiting. Using this teaching, numerous other liposomes comprising a CXCR4 antagonist and various lipid formulations can be produced by one of skill in the art.

Preparation and remote-loading of a silicasome comprising a CXCR4 antagonist can be accomplished in a similar manner. For example, the CXCR4 antagonist can be incorporated by trapping the CXR4 antagonist in the mesoporous interior of a nanparticle as described above.

This protocol also is illustrative and non-limiting. Using this teaching, numerous other silicasomes comprising a CXCR4 antagonist and various lipid formulations can be produced by one of skill in the art.

In this regard, it is noted that the lipid conjugation technology described herein can be used to make prodrugs out of chemo agents, which can be folded into a liposome. Thus, for example, ICD chemo agents like the taxanes can be incorporated into a phospholipid bilayer based on hydrophobicity, and this has been demonstrated for a MSNP where we used paclitaxel incorporation into the encapsulating phospholipid bilayer. The same can be done for a liposome.

These embodiments are illustrative and non-limiting. Using the teachings provided herein numerous variants will be available to one of skill in the art.

### Cargo Trapping Reagents.

As explained above, in certain embodiments a cargo-trapping reagent can be utilized to facilitate incorporation of a cargo (e.g., a CXCR4 antagonist) into the liposome or silicasome. The cargo-trapping reagent can be selected to interact with a desired cargo. In some embodiments, this interaction can be an ionic or protonation reaction, although other modes of interaction are contemplated. The cargo-trapping agent can have one or more ionic sites, i.e., can be mono-ionic or poly-ionic. The ionic moiety can be cationic, anionic, or in some cases, the cargo-trapping agent can include both cationic and anionic moieties. The ionic sites can be in equilibrium with corresponding uncharged forms; for example, an anionic carboxylate (—COO⁻) can be in equilibrium with its corresponding carboxylic acid (—COOH); or in another example, an amine (—NH₂) can be in equilibrium with its corresponding protonated ammonium form (—NH₃⁺). These equilibriums are influenced by the pH of the local environment.

Likewise, in certain embodiments, the cargo can include one or more ionic sites. In certain embodiments the cargo-trapping agent and cargo (e.g., CXCR4 antagonist) can be selected to interact inside the silicasome or liposome. This interaction can help retain the cargo within the nanoparticle until release of the cargo is desired. In some embodiments, the cargo can exist in a pH-dependent equilibrium between non-ionic and ionic forms. The non-ionic form can diffuse across the lipid bilayer and enter the vesicle or the pores of the MSNP. There, the cargo-trapping agent (e.g., a polyionic cargo-trapping agent) can interact with the ionic form of the cargo and thereby retain the cargo within the nanocarrier, e.g., within the vesicle or within the pores of the MSNP (provided the ionic forms of the cargo and cargo-trapping agent have opposite charges). In certain embodiments the interaction can be an ionic interaction and can include formation of a precipitate. Trapping of cargo within the liposome or silicasome can provide higher levels of cargo loading compared to similar systems, e.g., silicasomes that omit the cargo-trapping agent, or liposomes that do not include a trapping agent. Release of the cargo can be achieved by an appropriate change in pH to disrupt the interaction between the cargo and cargo-trapping agent, for example, by returning the cargo to its non-ionic state which can more readily diffuse across the lipid bilayer.

The cargo trapping agent need not be limited to TEA₈SOS. In certain embodiments the cargo trapping comprises small molecules like citric acid, (NH₄)₂SO₄, and the like. Other trapping agents include, but are not limited to, ammonium salts (e.g., ammonium sulfate, ammonium sucrose octasulfate, ammonium α-cyclodextrin sulfate, ammonium β-cyclodextrin sulfate, ammonium γ-cyclodextrin sulfate, ammonium phosphate, ammonium α-cyclodextrin phosphate, ammonium β-cyclodextrin phosphate, ammonium γ-cyclodextrin phosphate, ammonium citrate, ammonium acetate, and the like), trimethylammonium salts (e.g., trimethylammonium sulfate, trimethylammonium sucrose octasulfate, trimethylammonium α-cyclodextrin sulfate, trimethylammonium β-cyclodextrin sulfate, trimethylammonium γ-cyclodextrin sulfate, trimethylammonium phosphate, trimethylammonium α-cyclodextrin phosphate, trimethylammonium β-cyclodextrin phosphate, trimethylammonium γ-cyclodextrin phosphate, trimethylammonium citrate, trimethylammonium acetate, and the like), triethylammonium salts (e.g., triethylammonium sulfate, triethylammonium sucrose octasulfate, triethylammonium α-cyclodextrin sulfate, triethylammonium β-cyclodextrin sulfate, triethylammonium γ-cyclodextrin sulfate, triethylammonium phosphate, triethylammonium α-cyclodextrin phosphate, triethylammonium β-cyclodextrin phosphate, triethylammonium γ-cyclodextrin phosphate, triethylammonium citrate, triethylammonium acetate, and the like).

It is also worth pointing out that, in addition to TEA₈SOS, transmembrane pH gradients can also be generated by acidic buffers (e.g., citrate) (Chou et al. (2003) J. Biosci. Bioengineer., 95(4): 405-408; Nichols et al. (1976) Biochimica et Biophysica Acta (BBA)-Biomembranes, 455(1): 269-271), proton-generating dissociable salts (e.g. (NH₄)₂SO₄) (Haran et al. (1993) Biochimica et Biophysica Acta (BBA)-Biomembranes, 1151(2): 201-215; Maurer-Spurej et al. (1999) Biochimica et Biophysica Acta (BBA)-Biomembranes, 1416(1): 1-10; Fritze et al. (2006) Biochimica et Biophysica Acta (BBA)-Biomembranes, 1758(10): 1633-1640), or ionophore-mediated ion gradients from metal salts (e.g. A23187 and MnSO₄) (Messerer et al. (2004) Clinical Cancer Res. 10(19): 6638-6649; Ramsay et al. (2008) Eur. J. Pharmaceut. Biopharmaceut. 68(3): 607-617; Fenske et al. (1998) Biochimica et Biophysica Acta (BBA)-Biomembranes, 1414(1): 188-204). Moreover, it is possible to generate reverse pH gradients for drug loading, such as use a calcium acetate gradient to improve amphiphilic weak acid loading in LB-MSNP, a strategy that has been utilized in liposomes (Avnir et al. (2008) Arthritis & Rheumatism, 58(1): 119-129).

### Targeting Ligands and Immunoconjugates.

In certain embodiments the drug delivery vehicle the drug delivery vehicle described herein (e.g., silicasome or liposome comprising a CXCR4 antagonist) can be conjugated to one or more targeting ligands, e.g., to facilitate specific delivery in endothelial cells, to cancer cells, to fusogenic ligands, e.g., to facilitate endosomal escape, ligands to promote transport across the blood-brain barrier, and the like.

In one illustrative embodiment the targeting ligand can comprises a CXCR4 antagonist as described herein which can thereby target the drug delivery vehicle to cells displaying CXCR4 receptors.

In another illustrative, but non-limiting embodiment, the drug delivery vehicle described herein (e.g., silicasome or liposome comprising a CXCR4 antagonist) is conjugated to a fusogenic peptide such as histidine-rich H5WYG (H₂N-GLFHAIAHFIHGGWHGLIHGWYG-COOH, (SEQ ID NO:1)) (see, e.g., Midoux et al., (1998) Bioconjug. Chem. 9: 260-267).

In certain embodiments the drug delivery vehicle described herein (e.g., silicasome or liposome comprising a CXCR4 antagonist) is conjugated to one or more targeting ligand(s) that can include antibodies as well as targeting peptides. Targeting antibodies include, but are not limited to intact immunoglobulins, immunoglobulin fragments (e.g., F(ab)′₂, Fab, etc.) single chain antibodies, diabodies, affibodies, unibodies, nanobodies, and the like. In certain embodiments antibodies will be used that specifically bind a cancer marker (e.g., a tumor associated antigen). A wide variety of cancer markers are known to those of skill in the art. The markers need not be unique to cancer cells but can also be effective where the expression of the marker is elevated in a cancer cell (as compared to normal healthy cells) or where the marker is not present at comparable levels in surrounding tissues (especially where the chimeric moiety is delivered locally).

Illustrative cancer markers include, for example, the tumor marker recognized by the ND4 monoclonal antibody. This marker is found on poorly differentiated colorectal cancer, as well as gastrointestinal neuroendocrine tumors (see, e.g., Tobi et al. (1998) Cancer Detection and Prevention, 22(2): 147-152). Other important targets for cancer immunotherapy are membrane bound complement regulatory glycoproteins CD46, CD55 and CD59, which have been found to be expressed on most tumor cells in vivo and in vitro. Human mucins (e.g. MUC1) are known tumor markers as are gp100, tyrosinase, and MAGE, which are found in melanoma. Wild-type Wilms' tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CD5, CD7, CD19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin's disease has been characterized by the Leu-M1 marker. Various melanomas have been characterized by the HMB 45 marker. Non-Hodgkins lymphomas have been characterized by the CD20, CD19, and Ia marker. And various prostate cancers have been characterized by the PSMA and SE10 markers.

In addition, many kinds of tumor cells display unusual antigens that are either inappropriate for the cell type and/or its environment or are only normally present during the organisms' development (e.g., fetal antigens). Examples of such antigens include the glycosphingolipid GD2, a disialoganglioside that is normally only expressed at a significant level on the outer surface membranes of neuronal cells, where its exposure to the immune system is limited by the blood-brain barrier. GD2 is expressed on the surfaces of a wide range of tumor cells including neuroblastoma, medulloblastomas, astrocytomas, melanomas, small-cell lung cancer, osteosarcomas and other soft tissue sarcomas. GD2 is thus a convenient tumor-specific target for immunotherapies.

Other kinds of tumor cells display cell surface receptors that are rare or absent on the surfaces of healthy cells, and which are responsible for activating cellular signaling pathways that cause the unregulated growth and division of the tumor cell. Examples include (ErbB2) HER2/neu, a constitutively active cell surface receptor that is produced at abnormally high levels on the surface of breast cancer tumor cells.

Other useful targets include, but are not limited to CD20, CD52, CD33, epidermal growth factor receptor and the like.

An illustrative, but not limiting list of suitable tumor markers is provided in Table 3. Antibodies to these and other cancer markers are known to those of skill in the art and can be obtained commercially or readily produced, e.g., using phage-display technology. Such antibodies can readily be conjugated to the drug delivery nanocarrier (e.g., LB-coated nanoparticle) described herein, e.g., in the same manner that iRGD peptide is conjugated in Example 3.

TABLE 3
Illustrative cancer markers and associated references, all of which are incorporated
herein by reference for the purpose of identifying the referenced tumor markers.

| Marker | Reference |
|---|---|
| 5 alpha reductase | Délos et al. (1998) Int J Cancer, 75: 6 840-846 |
| α-fetoprotein | Esteban et al. (1996) Tumour Biol., 17(5): 299-305 |
| AM-1 | Harada et al. (1996) Tohoku J Exp Med., 180(3): 273-288 |
| APC | Dihlmannet al. (1997) Oncol Res., 9(3) 119-127 |
| APRIL | Sordat et al. (′998) J Exp Med., 188(6): 1185-1190 |
| BAGE | Böel et al. (1995) Immunity, 2: 167-175. |
| β-catenin | Hugh et al. (1999) Int J Cancer, 82(4): 504-11 |
| Bc12 | Koty et al. (1999) Lung Cancer, 23(2): 115-127 |
| bcr-abl (b3a2) | Verfaillie et al.(′996) Blood, 87(11): 4770-4779 |
| CA-125 | Bast et al. (′998) Int J Biol Markers, 13(4): 179-187 |
| CASP-8/FLICE | Mandruzzato et al. (1997) J Exp Med., 186(5): 785-793. |
| Cathepsins | Thomssen et al.(1995) Clin Cancer Res., 1(7): 741-746 |
| CD19 | Scheuermann et al. (1995) Leuk Lymphoma, 18(5-6): 385-397 |
| CD20 | Knox et al. (1996) Clin Cancer Res., 2(3): 457-470 |
| CD21, CD23 | Shubinsky et al. (1997) Leuk Lymphoma, 25(5-6): 521-530 |
| CD22, CD38 | French et al. (1995) Br J Cancer, 71(5): 986-994 |
| CD33 | Nakase et al. (1996) Am J Clin Pathol., 105(6): 761-768 |
| CD35 | Yamakawa et al. Cancer, 73(11): 2808-2817 |
| CD44 | Naot et al. (1997) Adv Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin Exp Immunol., 85(3): 418-423 |
| CD52 | Ginaldi et al. (1998) Leuk Res., 22(2): 185-191 |
| CD55 | Spendlove et al. (1999) Cancer Res., 59: 2282-2286. |
| CD59 (791Tgp72) | Jarvis et al. (1997) Int J Cancer, 71(6): 1049-1055 |
| CDC27 | Wang et al. (1999) Science, 284(5418): 1351-1354 |
| CDK4 | Wölfel et al. (1995) Science, 269(5228): 1281-1284 |
| CEA | Kass et al. (1999) Cancer Res., 59(3): 676-683 |
| c-myc | Watson et al. (1991) Cancer Res., 51(15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) J. Clin. Invest., 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J Biol Chem., 267(33): 24140-72414 |
| G250 | Divgi et al. (1998) Clin Cancer Res., 4(11): 2729-2739 |
| GAGE-Family | De Backer et al. (1999) Cancer Res., 59(13): 3157-3165 |
| gastrin 17 | Watson et al. (1995) Int J Cancer, 61(2): 233-240 |
| Gastrin-releasing | Wang et al. (1996) Int J Cancer, 68(4): 528-534 |
| hormone (bombesin) | |
| GD2/GD3/GM2 | Wiesner and Sweeley (1995) Int J Cancer, 60(3): 294-299 |
| GnRH | Bahk et al.(1998) Urol Res., 26(4): 259-264 |
| GnTV | Hengstler et al. (1998) Recent Results Cancer Res., 154: 47-85 |
| gp100/Pmel17 | Wagner et al. (1997) Cancer Immunol Immunother., 44(4): 239-247 |
| gp-100-in4 | Kirkin et al. (1998) APMIS, 106(7): 665-679 |
| gp15 | Maeurer et al.(1996) Melanoma Res., 6(1): 11-24 |
| gp75/TRP-1 | Lewis et al.(1995) Semin Cancer Biol., 6(6): 321-327 |
| hCG | Hoermann et al. (1992) Cancer Res., 52(6): 1520-1524 |
| Heparanase | Vlodavsky et al. (1999) Nat Med., 5(7): 793-802 |
| Her2/neu | Lewis et al. (1995) Semin Cancer Biol., 6(6): 321-327 |
| Her3 | |
| HMTV | Kahl et al.(1991) Br J Cancer, 63(4): 534-540 |
| Hsp70 | Jaattela et al. (1998) EMBO J., 17(21): 6124-6134 |
| hTERT | Vonderheide et al. (1999) Immunity, 10: 673-679. 1999. |
| (telomerase) | |
| IGFR1 | Ellis et al. (1998) Breast Cancer Res. Treat., 52: 175-184 |
| IL-13R | Murata et al. (1997) Biochem Biophys Res Commun., 238(1): 90-94 |
| iNOS | Klotz et al. (1998) Cancer, 82(10): 1897-1903 |
| Ki 67 | Gerdes et al. (1983) Int J Cancer, 31: 13-20 |
| KIAA0205 | Guéguen et al. (1998) J Immunol., 160(12): 6188-6194 |
| K-ras, H-ras, | Abrams et al. (1996) Semin Oncol., 23(1): 118-134 |
| N-ras | |
| KSA | Zhang et al. (1998) Clin Cancer Res., 4(2): 295-302 |
| (CO17-1A) | |
| LDLR-FUT | Caruso et al. (1998) Oncol Rep., 5(4): 927-930 |
| MAGE Family | Marchand et al. (1999) Int J Cancer, 80(2): 219-230 |
| (MAGE1, | |
| MAGE3, etc.) | |
| Mammaglobin | Watson et al. (1999) Cancer Res., 59: 13 3028-3031 |
| MAP17 | Kocher et al. (1996) Am J Pathol., 149(2): 493-500 |
| Melan-A/ | Lewis and Houghton (1995) Semin Cancer Biol., 6(6): 321-327 |
| MART-1 | |
| mesothelin | Chang et al. (1996) Proc. Natl. Acad. Sci., USA, 93(1): 136-140 |
| MIC A/B | Groh et al. (1998) Science, 279: 1737-1740 |
| MT-MMP's, such as | Sato and Seiki (1996) J Biochem (Tokyo), 119(2): 209-215 |
| MMP2, MMP3, | |
| MMP7, MMP9 | |
| Mox1 | Candia et al. (1992) Development, 116(4): 1123-1136 |
| Mucin, such as MUC- | Lewis and Houghton (1995) Semin Cancer Biol., 6(6): 321-327 |
| 1, MUC-2, MUC-3, | |
| and MUC-4 | |
| MUM-1 | Kirkin et al. (1998) APMIS, 106(7): 665-679 |
| NY-ESO-1 | Jager et al. (1998) J. Exp. Med., 187: 265-270 |
| Osteonectin | Graham et al. (1997) Eur J Cancer, 33(10): 1654-1660 |
| p15 | Yoshida et al. (1995) Cancer Res., 55(13): 2756-2760 |
| P170/MDR1 | Trock et al. (1997) J Natl Cancer Inst., 89(13): 917-931 |
| p53 | Roth et al. (1996) Proc. Natl. Acad. Sci., USA, 93(10): 4781-4786. |
| p97/melanotransferrin | Furukawa et al. (1989) J Exp Med., 169(2): 585-590 |
| PAI-1 | Grøndahl-Hansen et al. (1993) Cancer Res., 53(11): 2513-2521 |
| PDGF | Vassbotn et al. (1993) Mol Cell Biol., 13(7): 4066-4076 |
| Plasminogen (uPA) | Naitoh et al. (1995) Jpn J Cancer Res., 86(1): 48-56 |
| PRAME | Kirkin et al. (1998) APMIS, 106(7): 665-679 |
| Probasin | Matuo et al. (1985) Biochem Biophys Res Commun., 130(1): 293-300 |
| Progenipoietin | — |
| PSA | Sanda et al. (1999) Urology, 53(2): 260-266. |
| PSM | Kawakami et al.(1997) Cancer Res., 57(12): 2321-2324 |
| RAGE-1 | Gaugler et al.(1996) Immunogenetics, 44(5): 323-330 |
| Rb | Dosaka-Akita et al. (1997) Cancer, 79(7): 1329-1337 |
| RCAS1 | Sonoda et al.(1996) Cancer, 77(8): 1501-1509. |
| SART-1 | Kikuchi et al.(1999(Int J Cancer, 81(3): 459-466 |
| SSX gene | Gure et al. (1997) Int J Cancer, 72(6): 965-971 |
| Family | |
| STAT3 | Bromberg et al. (1999) Cell, 98(3): 295-303 |
| STn | Sandmaier et al. (1999) J Immunother., 22(1): 54-66 |
| (mucin assoc.) | |
| TAG-72 | Kuroki et al. (1990)Cancer Res., 50(16): 4872-4879 |
| TGF-α | Imanishi et al. (1989) Br J Cancer, 59(5): 761-765 |
| TGF-β | Picon et al. (1998) Cancer Epidemiol Biomarkers Prey, 7(6): 497-504 |
| Thymosin β 15 | Bao et al. (1996) Nature Medicine. 2(12), 1322-1328 |
| IFN-α | Moradi et al. (1993) Cancer, 72(8): 2433-2440 |
| TPA | Maulard et al. (1994) Cancer, 73(2): 394-398 |
| TPI | Nishida et al.(1984) Cancer Res 44(8): 3324-9 |
| TRP-2 | Parkhurst et al. (1998) Cancer Res., 58(21) 4895-4901 |
| Tyrosinase | Kirkin et al. (1998) APMIS, 106(7): 665-679 |
| VEGF | Hyodo et al. (1998) Eur J Cancer, 34(13): 2041-2045 |
| ZAG | Sanchez et al. (1999) Science, 283(5409): 1914-1919 |
| p16INK4 | Quelle et al. (1995) Oncogene Aug. 17, 1995; 11(4): 635-645 |
| Glutathione | Hengstler (1998) et al. Recent Results Cancer Res., 154: 47-85 |
| S-transferase | |

Any of the foregoing markers can be used as targets for the targeting moieties conjugated to the the drug delivery vehicle described herein (e.g., silicasome or liposome comprising a CXCR4 antagonist). In certain embodiments the target markers include, but are not limited to members of the epidermal growth factor family (e.g., HER2, HER3, EGF, HER4), CD1, CD2, CD3, CD5, CD7, CD13, CD14, CD15, CD19, CD20, CD21, CD23, CD25, CD33, CD34, CD38, 5E10, CEA, HLA-DR, HM 1.24, HMB 45, 1a, Leu-M1, MUC1, PMSA, TAG-72, phosphatidyl serine antigen, and the like.

The foregoing markers are intended to be illustrative and not limiting. Other tumor associated antigens will be known to those of skill in the art.

Where the tumor marker is a cell surface receptor, a ligand to that receptor can function as targeting moieties. Similarly, mimetics of such ligands can also be used as targeting moieties. Thus, in certain embodiments peptide ligands can be used in addition to or in place of various antibodies. An illustrative, but non-limiting list of suitable targeting peptides is shown in Table 4. In certain embodiments any one or more of these peptides can be conjugated to a drug delivery vehicle described herein.

TABLE 4
Illustrative, but non-limiting peptides that target membrane
receptors expressed or overexpressed by various cancer cells.

| Target Membrane | | SEQ ID |
|---|---|---|
| Receptor | Targeting Peptide | NO |
| Integrin receptor Aᵥβ₃ | c(RGDfK) | 2 |
| | c(RGDfC) | 3 |
| | c(RGDyC) | 4 |
| | RGD | |
| GFR | GE11 (YHWYGYTPQNVI) | 5 |
| GFR | GSG-KCCYSL | 6 |
| SSTR2 | Ostreotide | |
| GRP | QWAVGHML | 7 |
| CCK | DYMGWMDF | 8 |
| NT | RRPYIL | 9 |
| | RRPYILQLYENKPRRPYIL | 10 |
| LHRH | Gondaorelin | |
| GPRC family members | Antagonist G | |

c( ) indicates cyclopeptide.
Lower case indicates “D” amino acid.

In certain embodiments the drug delivery vehicle described herein (e.g., silicasome or liposome comprising a CXCR4 antagonist) can be conjugated to moieties that facilitate stability in circulation and/or that hide the drug delivery vehicle from the reticuloendothelial system (REC) and/or that facilitate transport across a barrier (e.g., a stromal barrier, the blood brain barrier, etc.), and/or into a tissue. In certain embodiments the drug delivery vehicles are conjugated to transferrin or ApoE to facilitate transport across the blood brain barrier. In certain embodiments the drug delivery vehicles are conjugated to folate.

Methods of coupling the drug delivery vehiclew described herein (e.g., silicasome or liposome comprising a CXCR4 antagonist) to targeting (or other) agents are well known to those of skill in the art. Examples include, but are not limited to the use of biotin and avidin or streptavidin (see, e.g., U.S. Pat. No. 4,885,172 A), by traditional chemical reactions using, for example, bifunctional coupling agents such as glutaraldehyde, diimide esters, aromatic and aliphatic diisocyanates, bis-p-nitrophenyl esters of dicarboxylic acids, aromatic disulfonyl chlorides and bifunctional arylhalides such as 1,5-difluoro-2,4-dinitrobenzene; p,p′-difluoro m,m′-dinitrodiphenyl sulfone, sulfhydryl-reactive maleimides, and the like. Appropriate reactions which may be applied to such couplings are described in Williams et al. Methods in Immunology and Immunochemistry Vol. 1, Academic Press, New York 1967. In one illustrative but non-limiting approach a peptide (e.g., iRGD) is coupled to the (e.g., ICD/IDO silicasome, ICD/IDO lipid vesicle, ICD-inducing nanomaterial carrier, etc.) by substituting a lipid (e.g., DSPE-PEG₂₀₀₀) with a lipid coupled to a linker (e.g., DSPE-PEG₂₀₀₀-maleimide), allowing thiol-maleimide coupling to the cysteine-modified peptide. It will also be recognized that in certain embodiments the targeting (and other) moieties can be conjugated to other moieties comprising the lipid bilayer on a silicasome or vesicle, or comprising the nanomaterial carrier. It is also possible to improve tumor delivery of the IDO inhibitor-ICD inducing nanoparticle, (e.g., OX laden IND-Lipid bilayer-MSNP (IND-LB-MSNP), MTX loaded Chol-IND-MSNP, etc.), through co-administration (not conjugated) of the iRGD peptide to enhance particle transcytosis.

The former conjugates and coupling methods are illustrative and non-limiting. Using the teachings provided herein, numerous other moieties can be conjugated to the drug delivery vehicle described herein (e.g., silicasome or liposome comprising a CXCR4 antagonist) by any of a variety of methods.

### Pharmaceutical Formulations, Administration and Therapy

In some embodiments, the drug delivery vehicles described herein (e.g., liposomes or silicasomes comprising one or more CXCR4 antagonist(s)) are administered alone or in a mixture with a physiologically-acceptable carrier (such as physiological saline or phosphate buffer) selected in accordance with the route of administration and standard pharmaceutical practice. For example, when used as an injectable, the drug delivery vehicles can be formulated as a sterile suspension, dispersion, or emulsion with a pharmaceutically acceptable carrier. In certain embodiments normal saline can be employed as the pharmaceutically acceptable carrier. Other suitable carriers include, e.g., water, buffered water, 0.4% saline, 0.3% glycine, 5% glucose and the like, including glycoproteins for enhanced stability, such as albumin, lipoprotein, globulin, etc. In compositions comprising saline or other salt-containing carriers, the carrier is preferably added following drug delivery vehicle formation. Thus, after the drug delivery vehicle is formed and loaded with suitable drug(s), the drug delivery vehicle can be diluted into pharmaceutically acceptable carriers such as normal saline.

The pharmaceutical compositions may be sterilized by conventional, well-known sterilization techniques. The resulting aqueous solutions, suspensions, dispersions, emulsions, etc., may be packaged for use or filtered under aseptic conditions. In certain embodiments the drug delivery drug delivery vehicles (e.g., LB-coated nanoparticles) are lyophilized, the lyophilized preparation being combined with a sterile aqueous solution prior to administration. The compositions may also contain pharmaceutically acceptable auxiliary substances as required to approximate physiological conditions, such as pH-adjusting and buffering agents, tonicity adjusting agents and the like, for example, sodium acetate, sodium lactate, sodium chloride, potassium chloride, calcium chloride, etc.

Additionally, in certain embodiments, the pharmaceutical formulation(s) may include lipid-protective agents that protect lipids against free-radical and lipid-peroxidative damage on storage. Lipophilic free-radical quenchers, such as alpha-tocopherol and water-soluble iron-specific chelators, such as ferrioxamine, are suitable.

The concentration of the drug delivery vehicle (e.g., liposomes or silicasomes comprising one or more CXCR4 antagonist(s)) in the pharmaceutical formulations can vary widely, e.g., from less than approximately 0.05%, usually at least approximately 2 to 5% to as much as 10 to 50%, or to 40%, or to 30% by weight and are selected primarily by fluid volumes, viscosities, etc., in accordance with the particular mode of administration selected. For example, the concentration may be increased to lower the fluid load associated with treatment. This may be particularly desirable in patients having atherosclerosis-associated congestive heart failure or severe hypertension. Alternatively, drug delivery vehicles composed of irritating lipids may be diluted to low concentrations to lessen inflammation at the site of administration. The amount of drug delivery vehicles administered will depend upon the particular drug(s) used, the disease state being treated and the judgment of the clinician but will generally be between approximately 0.01 and approximately 50 mg per kilogram of body weight, preferably between approximately 0.1 and approximately 5 mg per kg of body weight.

In some embodiments, e.g., it is desirable to include polyethylene glycol (PEG)-modified phospholipids in the drug delivery vehicles described herein. Alternatively, or additionally, in certain embodiments, PEG-ceramide, or ganglioside G_{MI}-modified lipids can be incorporated in the drug delivery vehicle (e.g., liposomes or silicasomes comprising one or more CXCR4 antagonist(s)). Addition of such components helps prevent drug delivery vehicle aggregation and provides for increasing circulation lifetime and increasing the delivery of the loaded drug delivery vehicles to the target tissues. In certain embodiments the concentration of the PEG-modified phospholipids, PEG-ceramide, or G_{MI}-modified lipids in the drug delivery vehicles will be approximately 1 to 15%.

In some embodiments, overall drug delivery vehicle charge is an important determinant in drug delivery vehicle clearance from the blood. It is believed that highly charged drug delivery vehicles (i.e. zeta potential >+35 mV) will be typically taken up more rapidly by the reticuloendothelial system (see, e.g., Juliano (1975) Biochem. Biophys. Res. Commun. 63: 651-658 discussing liposome clearance by the RES) and thus have shorter half-lives in the bloodstream. Drug delivery vehicles with prolonged circulation half-lives are typically desirable for therapeutic uses. For instance, in certain embodiments, drug delivery vehicles (e.g., liposomes or silicasomes comprising one or more CXCR4 antagonist(s)) that are maintained from 8 hrs, or 12 hrs, or 24 hrs, or greater are desirable.

In another example of their use, the drug delivery vehicles described herein (e.g., liposomes or silicasomes comprising one or more CXCR4 antagonist(s)) can be incorporated into a broad range of topical dosage forms including but not limited to gels, oils, emulsions, and the like, e.g., for the treatment of a topical cancer. For instance, in some embodiments the suspension containing the drug delivery vehicle is formulated and administered as a topical cream, paste, ointment, gel, lotion, and the like.

In some embodiments, pharmaceutical formulations comprising the drug delivery vehicles described herein (e.g., liposomes or silicasomes comprising one or more CXCR4 antagonist(s)) additionally incorporate a buffering agent. The buffering agent may be any pharmaceutically acceptable buffering agent. Buffer systems include, but are not limited to citrate buffers, acetate buffers, borate buffers, and phosphate buffers. Examples of buffers include, but are not limited to citric acid, sodium citrate, sodium acetate, acetic acid, sodium phosphate and phosphoric acid, sodium ascorbate, tartaric acid, maleic acid, glycine, sodium lactate, lactic acid, ascorbic acid, imidazole, sodium bicarbonate and carbonic acid, sodium succinate and succinic acid, histidine, and sodium benzoate, benzoic acid, and the like.

In some embodiments, pharmaceutical formulations comprising the drug delivery vehicles described herein (e.g., liposomes or silicasomes comprising one or more CXCR4 antagonist(s)) additionally incorporate a chelating agent. The chelating agent may be any pharmaceutically acceptable chelating agent. Chelating agents include but are not limited to ethylene diaminetetraacetic acid (also synonymous with EDTA, edetic acid, versene acid, and sequestrene), and EDTA derivatives, such as dipotassium edetate, disodium edetate, edetate calcium disodium, sodium edetate, trisodium edetate, and potassium edetate. Other chelating agents include citric acid (e.g., citric acid monohydrate) and derivatives thereof. Derivatives of citric acid include anhydrous citric acid, trisodiumcitrate-dihydrate, and the like. Still other chelating agents include, but are not limited to, niacinamide and derivatives thereof and sodium deoxycholate and derivatives thereof.

In some embodiments, pharmaceutical formulations comprising the drug delivery vehicles described herein (e.g., liposomes or silicasomes comprising one or more CXCR4 antagonist(s)) additionally incorporate an antioxidant. The antioxidant may be any pharmaceutically acceptable antioxidant. Antioxidants are well known to those of ordinary skill in the art and include, but are not limited to, materials such as ascorbic acid, ascorbic acid derivatives (e.g., ascorbylpalmitate, ascorbylstearate, sodium ascorbate, calcium ascorbate, etc.), butylated hydroxy anisole, buylated hydroxy toluene, alkylgallate, sodium meta-bisulfate, sodium bisulfate, sodium dithionite, sodium thioglycollic acid, sodium formaldehyde sulfoxylate, tocopherol and derivatives thereof, (d-alpha tocopherol, d-alpha tocopherol acetate, dl-alpha tocopherol acetate, d-alpha tocopherol succinate, beta tocopherol, delta tocopherol, gamma tocopherol, and d-alpha tocopherol polyoxyethylene glycol 1000 succinate) monothioglycerol, sodium sulfite and N-acetyl cysteine. In certain embodiments such materials, when present, are typically added in ranges from 0.01 to 2.0%.

In some embodiments, pharmaceutical formulations comprising the drug delivery vehicles described herein (e.g., liposomes or silicasomes comprising one or more CXCR4 antagonist(s)) are formulated with a cryoprotectant. The cryoprotecting agent may be any pharmaceutically acceptable cryoprotecting agent. Common cryoprotecting agents include, but are not limited to, histidine, polyethylene glycol, polyvinyl pyrrolidine, lactose, sucrose, mannitol, polyols, and the like.

In some embodiments, pharmaceutical formulations comprising the drug delivery vehicles described herein (e.g., liposomes or silicasomes comprising one or more CXCR4 antagonist(s)) are formulated with an isotonic agent. The isotonic agent can be any pharmaceutically acceptable isotonic agent. This term is used in the art interchangeably with iso-osmotic agent and is known as a compound that is added to the pharmaceutical preparation to increase the osmotic pressure, e.g., in some embodiments to that of 0.9% sodium chloride solution, which is iso-osmotic with human extracellular fluids, such as plasma. Illustrative isotonicity agents include, but are not limited to, sodium chloride, mannitol, sorbitol, lactose, dextrose and glycerol.

In certain embodiments pharmaceutical formulations of the drug delivery vehicles described herein may optionally comprise a preservative. Common preservatives include, but are not limited to those selected from the group consisting of chlorobutanol, parabens, thimerosol, benzyl alcohol, and phenol. Suitable preservatives include but are not limited to: chlorobutanol (e.g., 0.3-0.9% w/v), parabens (e.g., 0.01-5.0%), thimerosal (e.g., 0.004-0.2%), benzyl alcohol (e.g., 0.5-5%), phenol (e.g., 0.1-1.0%), and the like.

In some embodiments, pharmaceutical formulations comprising the drug delivery vehicles described herein are formulated with a humectant, e.g., to provide a pleasant mouth-feel in oral applications. Humectants known in the art include, but are not limited to, cholesterol, fatty acids, glycerin, lauric acid, magnesium stearate, pentaerythritol, and propylene glycol.

In some embodiments, an emulsifying agent is included in the formulations, for example, to ensure complete dissolution of all excipients, especially hydrophobic components such as benzyl alcohol. Many emulsifiers are known in the art, e.g., polysorbate 60.

For some embodiments related to oral administration, it may be desirable to add a pharmaceutically acceptable flavoring agent and/or sweetener. Compounds such as saccharin, glycerin, simple syrup, and sorbitol are useful as sweeteners.

### Administration

The drug delivery vehicles described herein (e.g., liposomes or silicasomes comprising one or more CXCR4 antagonist(s)) can be administered to a subject (e.g., patient) by any of a variety of techniques.

In certain embodiments the drug delivery vehicles described herein and/or pharmaceutical formulations thereof are administered parenterally, e.g., intraarticularly, intravenously, intraperitoneally, subcutaneously, or intramuscularly. In some embodiments, the pharmaceutical compositions are administered intravenously, intraarteraly, or intraperitoneally by a bolus injection (see, e.g., U.S. Pat. Nos. 3,993,754; 4,145,410; 4,235,871; 4,224,179; 4,522,803; and 4,588,578 describing administration of liposomes). Particular pharmaceutical formulations suitable for this administration are found in Remington's Pharmaceutical Sciences, Mack Publishing Company, Philadelphia, Pa., 17th ed. (1985). Typically, the formulations comprise a solution of the drug delivery drug delivery vehicle suspended in an acceptable carrier, preferably an aqueous carrier. As noted above, suitable aqueous solutions include, but are not limited to physiologically compatible buffers such as Hanks solution, Ringer's solution, or physiological (e.g., 0.9% isotonic) saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. These compositions may be sterilized by conventional, well-known sterilization techniques, or may be sterile filtered. The resulting aqueous solutions may be packaged for use as is, or lyophilized, the lyophilized preparation being combined with a sterile aqueous solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc., e.g., as described above.

In other methods, the pharmaceutical formulations containing the drug delivery vehicles described herein may be contacted with the target tissue by direct application of the preparation to the tissue. The application may be made by topical, “open” or “closed” procedures. By “topical” it is meant the direct application of the pharmaceutical preparation to a tissue exposed to the environment, such as the skin, oropharynx, external auditory canal, and the like. Open procedures are those procedures that include incising the skin of a patient and directly visualizing the underlying tissue to which the pharmaceutical formulations are applied. This is generally accomplished by a surgical procedure, such as a thoracotomy to access the lungs, abdominal laparotomy to access abdominal viscera, or other direct surgical approaches to the target tissue. Closed procedures are invasive procedures in which the internal target tissues are not directly visualized but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrizamide imaging of the spinal cord. Alternatively, the preparations may be administered through endoscopic devices. In certain embodiments the pharmaceutical formulations are introduced via a cannula.

In certain embodiments the pharmaceutical formulations comprising the drug delivery vehicles described herein are administered via inhalation (e.g., as an aerosol). Inhalation can be a particularly effective delivery route for administration to the lungs and/or to the brain. For administration by inhalation, the drug delivery drug delivery vehicles are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In certain embodiments, the drug delivery vehicles described herein (e.g., liposomes or silicasomes comprising one or more CXCR4 antagonist(s)) are formulated for oral administration. For oral administration, suitable formulations can be readily formulated by combining the drug delivery drug delivery vehicles) with pharmaceutically acceptable carriers suitable for oral delivery well known in the art. Such carriers enable the active agent(s) described herein to be formulated as tablets, pills, dragees, caplets, lozenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents, and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric coated using standard techniques. The preparation of enteric-coated particles is disclosed for example in U.S. Pat. Nos. 4,786,505 and 4,853,230.

In various embodiments the drug delivery vehicles described herein can be formulated in rectal or vaginal compositions such as suppositories or retention enemas, e.g., containing conventional suppository bases such as cocoa butter or other glycerides. Methods of formulating active agents for rectal or vaginal delivery are well known to those of skill in the art (see, e.g., Allen (2007) Suppositories, Pharmaceutical Press) and typically involve combining the active agents with a suitable base (e.g., hydrophilic (PEG), lipophilic materials such as cocoa butter or Witepsol W45), amphiphilic materials such as Suppocire AP and polyglycolized glyceride, and the like). The base is selected and compounded for a desired melting/delivery profile.

The route of delivery of the drug delivery vehicles described herein can also affect their distribution in the body. Passive delivery of drug delivery vehiclea (e.g., liposomes or silicasomes comprising one or more CXCR4 antagonist(s)) involves the use of various routes of administration e.g., parenterally, although other effective administration forms, such as intraarticular injection, inhalant mists, orally active formulations, transdermal iontophoresis, or suppositories are also envisioned. Each route produces differences in localization of the drug delivery drug delivery vehicle.

Because dosage regimens for pharmaceutical agents are well known to medical practitioners, the amount of the liposomal pharmaceutical agent formulations that is effective or therapeutic for the treatment of a disease or condition in mammals and particularly in humans will be apparent to those skilled in the art. The optimal quantity and spacing of individual dosages of the formulations herein will be determined by the nature and extent of the condition being treated, the form, route and site of administration, and the particular patient being treated, and such optima can be determined by conventional techniques. It will also be appreciated by one of skill in the art that the optimal course of treatment, e.g., the number of doses given per day for a defined number of days, can be ascertained by those skilled in the art using conventional course of treatment determination tests.

Typically, the drug delivery vehicles described herein and/or pharmaceutical formations thereof described herein are used therapeutically in animals (including man) in the treatment of various cancers. In certain embodiments the drug delivery vehicles and/or pharmaceutical formations thereof described herein are particularly well suited in conditions that require: (1) repeated administrations; and/or (2) the sustained delivery of the drug in its bioactive form; and/or (3) the decreased toxicity with suitable efficacy compared with the free drug(s) in question. In various embodiments the drug delivery vehicles and/or pharmaceutical formations thereof are administered in a therapeutically effective dose. The term “therapeutically effective” as it pertains to the drug delivery vehicles described herein and formulations thereof means that the combination of ICD inducer and IDO inhibitor produces a desirable effect on the cancer. Such desirable effects include but are not limited to slowing and/or stopping tumor growth and/or proliferation and/or slowing and/or stopping proliferation of metastatic cells, reduction in size and/or number of tumors, and/or elimination of tumor cells and/or metastatic cells, and/or prevention of recurrence of the cancer following remission.

Exact dosages will vary depending upon such factors as the particular ICD inducer(s) and IDO inhibitors and the desirable medical effect, as well as patient factors such as age, sex, general condition, and the like. Those of skill in the art can readily take these factors into account and use them to establish effective therapeutic concentrations without resort to undue experimentation.

For administration to humans (or to non-human mammals) in the curative, remissive, retardive, or prophylactic treatment of diseases the prescribing physician will ultimately determine the appropriate dosage of the drug for a given human (or non-human) subject, and this can be expected to vary according to the age, weight, and response of the individual as well as the nature and severity of the patient's disease. In certain embodiments the dosage of the drug provided by the drug delivery vehicle(s) can be approximately equal to that employed for the free drug. However as noted above, the drug delivery vehicles described herein can significantly reduce the toxicity of the drug(s) administered thereby and significantly increase a therapeutic window. Accordingly, in some cases dosages in excess of those prescribed for the free drug(s) will be utilized.

In certain embodiments, the dose of each of the drug(s) (e.g., ICD inducer, IDO inhibitor) administered at a particular time point will be in the range from about 1 to about 1,000 mg/m²/day, or to about 800 mg/m²/day, or to about 600 mg/m²/day, or to about 400 mg/m²/day. For example, in certain embodiments a dosage (dosage regiment) is utilized that provides a range from about 1 to about 350 mg/m²/day, 1 to about 300 mg/m²/day, 1 to about 250 mg/m²/day, 1 to about 200 mg/m²/day, 1 to about 150 mg/m²/day, 1 to about 100 mg/m²/day, from about 5 to about 80 mg/m²/day, from about 5 to about 70 mg/m²/day, from about 5 to about 60 mg/m²/day, from about 5 to about 50 mg/m²/day, from about 5 to about 40 mg/m²/day, from about 5 to about 20 mg/m²/day, from about 10 to about 80 mg/m²/day, from about 10 to about 70 mg/m²/day, from about 10 to about 60 mg/m²/day, from about 10 to about 50 mg/m²/day, from about 10 to about 40 mg/m²/day, from about 10 to about 20 mg/m²/day, from about 20 to about 40 mg/m²/day, from about 20 to about 50 mg/m²/day, from about 20 to about 90 mg/m²/day, from about 30 to about 80 mg/m²/day, from about 40 to about 90 mg/m²/day, from about 40 to about 100 mg/m²/day, from about 80 to about 150 mg/m²/day, from about 80 to about 140 mg/m²/day, from about 80 to about 135 mg/m²/day, from about 80 to about 130 mg/m²/day, from about 80 to about 120 mg/m²/day, from about 85 to about 140 mg/m²/day, from about 85 to about 135 mg/m²/day, from about 85 to about 135 mg/m²/day, from about 85 to about 130 mg/m²/day, or from about 85 to about 120 mg/m²/day. In certain embodiments the does administered at a particular time point may also be about 130 mg/m²/day, about 120 mg/m²/day, about 100 mg/m²/day, about 90 mg/m²/day, about 85 mg/m²/day, about 80 mg/m²/day, about 70 mg/m²/day, about 60 mg/m²/day, about 50 mg/m²/day, about 40 mg/m²/day, about 30 mg/m²/day, about 20 mg/m²/day, about 15 mg/m²/day, or about 10 mg/m²/day.

Dosages may also be estimated using in vivo animal models, as will be appreciated by those skill in the art.

The dose administered may be higher or lower than the dose ranges described herein, depending upon, among other factors, the bioavailability of the composition, the tolerance of the individual to adverse side effects, the mode of administration and various factors discussed above. Dosage amount and interval may be adjusted individually to provide plasma levels of the composition that are sufficient to maintain therapeutic effect, according to the judgment of the prescribing physician. Skilled artisans will be able to optimize effective local dosages without undue experimentation in view of the teaching provided herein.

Multiple doses (e.g., continuous or bolus) of the compositions as described herein may also be administered to individuals in need thereof of the course of hours, days, weeks, or months. For example, but not limited to, 1, 2, 3, 4, 5, or 6 times daily, every other day, every 10 days, weekly, monthly, twice weekly, three times a week, twice monthly, three times a month, four times a month, five times a month, every other month, every third month, every fourth month, etc.

### Methods of Treatment.

In various embodiments methods of treatment using drug delivery vehicles described herein (e.g., liposomes or silicasomes comprising one or more CXCR4 antagonist(s)) and/or pharmaceutical formulation(s) comprising the drug delivery vehicles described herein are provided. In certain embodiments the method(s) comprise a method of treating a cancer. In certain embodiments the method can comprise administering to a subject in need thereof an effective amount of a drug delivery vehicle described herein (e.g., liposomes or silicasomes comprising one or more CXCR4 antagonist(s)), and/or a pharmaceutical formulation comprising the drug delivery vehicle(s).

In certain embodiments the drug delivery vehicles described herein (e.g., liposomes or silicasomes comprising one or more CXCR4 antagonist(s)) and/or pharmaceutical formulations thereof are used as a primary therapy in a chemotherapeutic regimen. In certain embodiments the drug delivery vehicles and/or pharmaceutical formulations thereof are component(s) in an adjunct therapy in addition to chemotherapy using one or more other chemotherapeutic agents, and/or surgical resection of a tumor mass, and/or radiotherapy.

In certain embodiments the drug delivery vehicles described herein and/or pharmaceutical formulations thereof are components in a multi-drug chemotherapeutic regimen. In certain embodiments the multi-drug chemotherapeutic regimen comprises at least two drugs selected from the group consisting of irinotecan (IRIN), oxaliplatin (OX), 5-fluorouracil (5-FU), and leucovorin (LV). In certain embodiments the multi-drug chemotherapeutic regimen comprises at least three drugs selected from the group consisting of irinotecan (IRIN), oxaliplatin (OX), 5-fluorouracil (5-FU), and leucovorin (LV). In certain embodiments the multi-drug chemotherapeutic regimen comprises at least irinotecan (IRIN), oxaliplatin (OX), 5-fluorouracil (5-FU), and leucovorin (LV).

In various embodiments the drug delivery vehicles described herein and/or pharmaceutical formulation(s) thereof are effective for treating any of a variety of cancers. In certain embodiments the cancer is pancreatic ductal adenocarcinoma (PDAC). In certain embodiments the cancer is a cancer selected from the group consisting of acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, glioblastoma, brain and spinal cord tumors, brain stem glioma, central nervous system atypical teratoid/rhabdoid tumor, central nervous system embryonal tumors, central nervous system germ cell tumors, craniopharyngioma, ependymoma, breast cancer, bronchial tumors, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, chronic myeloid leukemia (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

In certain embodiments the cancer is breast cancer. In certain embodiments the cancer is triple negative breast cancer.

In certain embodiments the drug delivery vehicles described herein are co-administered with one or more additional drugs. In certain embodiments the additional drugs comprise one or more drugs selected from the group consisting of an IDO inhibitor, an immunogenic cell death (ICD)-inducing drug.

In various embodiments of these treatment methods, the drug delivery vehicles described herein and/or pharmaceutical formulations thereof are administered via a route selected from the group consisting of intravenous administration, intraarterial administration, intracerebral administration, intrathecal administration, oral administration, aerosol administration, administration via inhalation (including intranasal and intratracheal delivery, intracranial administration via a cannula, and subcutaneous or intramuscular depot deposition. In certain embodiments the nanocarrier and/or pharmaceutical formulation is administered as an injection, from an IV drip bag, or via a drug-delivery cannula. In various embodiments the subject is a human and in other embodiments the subject is a non-human mammal.

### Kits

In certain embodiments, kits are provided containing reagents for the practice of any of the methods described herein. In certain embodiments the kit comprises a container containing one or more of the drug delivery vehicles described herein (e.g., liposomes or silicasomes comprising one or more CXCR4 antagonist(s)).

In certain embodiments the kit can contain additional drugs for co-administration with the drug delivery vehicles described herein. In certain embodiments the additional drugs comprise one or more IDO inhibitors, immune checkpoint inhibitors (ICIs), immunogenic cell death-(ICD)-inducing drugs, GSK3 inhibitors, and the like, e.g., as

Additionally, in certain embodiments, the kits can include instructional materials disclosing the means of the use of the drug delivery vehicles described herein as a therapeutic for a cancer (e.g., breast cancer, pancreatic cancer, gastric cancer, cervical cancer, ovarian cancer, etc.).

In addition, the kits optionally include labeling and/or instructional materials providing directions (e.g., protocols) for the use of the materials described herein, e.g., alone or in combination for the treatment of various cancers. Instructional materials can also include recommended dosages, description(s) of counterindications, and the like.

While the instructional materials in the various kits typically comprise written or printed materials, they are not limited to such. Any medium capable of storing such instructions and communicating them to an end user is contemplated by this invention. Such media include but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Fabrication and Evaluation of Liposomes and Silicasomes Comprising a CXCR4 Antagonist

Our approach has been to determine if the weak basic properties of the CXCR4 antagonists AMD3100, AMD3465 and AMD11070 could be used to achieve remote loading in lipid bilayer (LB) carriers (e.g., silicasomes and liposomes) using trapping agents such as TEA8SOS or ammonium sulfate (FIG. 8). Plerixafor (AMD 3100) is an immunostimulant used to mobilize hematopoietic stem cells from the bone marrow into the bloodstream in cancer patients. The stem cells are then extracted from the blood and transplanted back to the patient. The drug was developed by AnorMED, which was subsequently bought by Genzyme. Mavorixafor (AMD-11070) is a small molecule drug candidate that belongs to a new investigational class of anti-HIV drugs known as entry (fusion) inhibitors. AMD-11070 has been studied in Phase I/II clinical trials for the treatment of renal cell carcinoma and Phase I clinical trials for the treatment of malignant melanoma and solid tumors. AMD3465 is a potent antagonist of CXCR4, and potently inhibits the replication of X4 HIV strains (IC50: 1-10 nM).

These drugs were chosen for their inhibitory potency of the activity of the CXCR4 chemokine receptor, size, solubility and pKa values, predicting the possibility of remote loading by the protonating agents such as sucrose sulfate and ammonium sulfate.

We utilized the weak-basic properties of a selected series of CXCR4 inhibitors (AMD3100, AMD3465, and AMD11070) to assess drug loading capacities across liposomal lipid bilayers. This was accomplished using triethylammonium sucrose octasulfate (TEA₈SOS) or ammonium sulfate to achieve loading capacities of 8-20% in liposomes. The protocol is outlined in FIG. 9, which shows that the lipid composition was comprised of DSPC:Chol:PEG2000 in a molar ratio of 3:2:0.15, using a soaking of the lipid biofilm in the trapping agent, before sonication, extrusion and removal of the nonencapsulated trapping agents over a PD-10 column. This was followed by incubating the liposome suspension in the suspended CXCR4 antagonists using a feed ratio of 20%. The non-incorporated drug was removed by a PD-10 column. The liposomes were characterized by CryoEM as well as in a zetasizer to determine particle size, PDI and zeta potential, as shown in FIG. 10. The drug encapsulation efficiency and loading capacity were also determined for each of the liposomes made using both protonating agents, as shown in Table 5.

TABLE 5
Loading of CXCR4 antagonists in liposomes at a drug
feed ration of 20% (drug:lipids w/w). Liposome is
DSPC:Chol:DSPE-PEG2000 in 3:2:0.15 molar ratio.

| | |
|---|---|
| (NH₄)₂SO₄ | TEA8SOS |

| | EE % | LC % | EE % | LC % |
|---|---|---|---|---|
| AMD3100 | 19% | 3.8% | 42.4% | 8.5% |
| AMD3465 | 68.4% | 13.7% | 37.4% | 7.5% |
| AMD070 | 55.8% | 11.2% | 84.3% | 16.9% |

The best loading capacity (LC) in liposomes, was for AMD11070 (LC=17%), prompting construction of an AMD11070-silicasome (FIGS. 9-10, and Table 5).

The bare MSNPs were synthesized and purified as described by Liu et al., (2016) ACS Nano, 10: 2, 2702-2715). More specifically the protocol was as follows:

Lipids, available as stock solutions from Avanti, were purchased as solution in CHCl3, normally sold at 25 mg/mL If powders were purchased, such as cholesterol, stock solutions were made in house in glass vails that can withhold CHCl₃. Typically, the stock solution was diluted into 5 mg/mL in CHCl3 for more accurate lipid transfer by 1 mL syringes.

The solubility of lipids such as DPPC/DSPC in CHCl3 is temperature sensitive. When stored at −20° C. the stock solution is a suspension that only becomes freely soluble when warmed up to room temperature. Accordingly, all lipid stock solutions were warmed up and vortexed to dissolve all precipitated lipids before dispersion.

For short-term use, lipids or lipid-blends of various components with low solubilities in CHCl₃ (such as single-chain lyso-lipids or cholesteryl-conjugated drugs etc.) can be prepared in CHCl₃:MeOH=4:1 (v/v) to greatly improve the solubility at a typical concentration of 5 mg/mL However, stock solutions containing alcohols are preferably consumed as soon as possible to avoid transesterification between the lipid and the alcohol resulting in lipid degradation.

To transfer the lipid stock solutions for formulation, use only gas-tight glass syringes with 2-inch metal needles (manually bent for easier handling). Plastic pipettes are not used for lipid transfer especially when organic solvents are present. Doing so results in contamination of plastic/plasticizer and resulting in irreproducible formulations and alters the release profiles and/or stability.

To transfer the lipids, glass syringes are washed with soap and warm water, rinse with diH₂O followed by acetone then dried by dust-free nitrogen before use. It was important to have a separated syringe-set for lipids and cholesterol because the latter is much harder to clean and contamination can cause significant changes in liposomal properties. It is also important to wash the syringes with acetone after lipid transfer to avoid lipid contamination before transferring the next lipid. It was also important to transfer standard lipids first before transferring PEGylated lipids. Syringes were properly cleaned after transferring PEGylated lipids to avoid syringe blockade when dried. If that happened, the blocked syringe was placed in acetone with bath sonication to unblock the syringe.

The first step of making liposomes was to make a lipid film in a round bottom flask. The flask was pre-cleaned by warm water with soap, diH₂O and then washed thoroughly with acetone to remove any residual lipids/surfactants/detergents that will either contaminate or destroy the liposomes.

With the cleaned round bottom flask (50/100 mL flask for lipid batch size <30 mg), lipid stock solutions were transferred into the flask according to the formulation chart. Additional CHCl3 was added to increase the lipid solution volume to 5-10 m. The round bottom flask is then transferred to the rotary evaporator with a solvent-trap adaptor and clip-secured.

The lipid film was made by gradually evaporating the CHCl3 under vacuum. The evaporation process needs to be carefully monitored at a evaporation speed without causing the solvent bubbling. Typical conditions for evaporation were water batch 40-45° C., r.p.m.=150-170 for 50/100/mL round bottom flask (larger 250 mL flask at 70-100 r.p.m.).

The vacuum was turned to the maximum when no solvents were visible in the rotating flask and the evaporation was continued for 10 more minutes. Then the flask was removed to inspect the lipid film under light.

The ideal lipid film should be thin, with even coating of the lower half of the round bottom flask without seeing any aggregation occurs. This requires that all lipid compositions need to be precipitated out evenly while evaporating the solvent. Unevenly coated lipid film (sometimes with aggregation or crystals precipitations) can result from sequential precipitation of the formulation components, which results in phase-separated formulation and uneven liposome formation (for example some liposomes have higher X component some have lower resulting in different drug release and stability etc).

Once an evenly coated lipid film was obtained. Flush the round bottom flask with dust-free nitrogen for 3-5 min to dry up the film completely. If the film became unevenly coated when completed dried up, the film was dissolved with CHCl₃ and the film making process was repeated.

Before hydrating the lipid film, an (NH₄)₂SO₄ solution was prepared at 240 mM (37.71 g/L) with a pH around 5.4 without further adjustment. The 240 mM (NH₄)₂SO₄ solution was filtered using a 0.45 μm syringe filter into a pre-cleaned Duran bottle (washed, cleaned with diH2O and acetone then dried) as stock solution. The (NH₄)₂SO₄ was filtered again through a 0.22 μm syringe filter for lipid film hydration when needed (and for DLS size measurement) in sterile cell culture grade centrifuge tubes.

To hydrate the film, the 0.22 μm filtered (NH₄)₂SO₄ solution was transferred into the round bottom flask (1-2 mL according to the formulation chart shown in Table 6, below using a P1000 pipette. The round bottom flask was flushed with N2 and sealed with parafilm. Sealing the liposomal dispersion (aqueous) with N₂ to delays lipid oxidation, either during the lipid film hydration with heating or later on during storage after extrusion.

TABLE 6
Formulation parameters.

| | | | Lipid | Lipid | Lipid | Stock | Stock |
|---|---|---|---|---|---|---|---|
| | | | Conc | quantity | wt | Cong | Vol. |
| Lipid | MW | Molar % | (mM) | (μmol) | (mg) | (mg/mL) | (μL) |
| DSPC | 790.15 | 55 | 3.85 | 7.70 | 6.1 | 5 | 1217 |
| Chol | 386.00 | 40 | 2.80 | 5.60 | 2.2 | 5 | 432 |
| DSPE- | 2805.50 | 5 | 0.35 | —/7-0 | 2.0 | 5 | 393 |
| PEG2kDa | | | | | | | |
| total | | 100 | 7.00 | 14.00 | 10.2 | | 2042 |
| DSPC | 790.15 | 55 | 3.85 | 11.55 | 9.1 | 5 | 1825 |
| Chol | 386.00 | 40 | 2.80 | 8.40 | 3.2 | 5 | 649 |
| DSPE- | 2805.50 | 5 | 0.35 | 0.35 | 2.9 | 5 | 589 |
| PEG2kDa | | | | | | | |
| total | | 100 | 7.00 | 7.00 | 15.3 | | 3063 |

In realty to accommodate for potential loss due to leakage during the extrusion process, lipids are prepared with 25-50% extra. For example, the same formulation prepared at 150% (50% extra) = 7 mM lipids × 3 mL.

Once the flask was sealed, the lipid film and the (NH4)2SO4 solution was heated at 65° C. for 5 minutes in the water bath and then vortexed to form a milky suspension. The heating and vortexing was repeated as many times as needed to fully resuspend the lipid film.

Liposome extrusion was performed using an extruder as described at //avantilipids.com/divisions/equipment-products/mini-extruder-extrusion-technique.

In brief, vortexed liposomal dispersions were first extruded through 1000 nm filter (5 cycles+1=11 times, back and forth=1 cycle) to remove dust. The plus 1 stroke after 5 cycles resulted in the liposomes ending up in the opposite side of the extruder or the other side of the filter, which is the filtered side.

The extruded liposomes, if more than 1 mL, were be stored temporary in a 50 mL tube according to the extruded sizes, for example 1000 nm at this stage. The liposomes continued to be sequentially extruded through 800 nm (5 cycles+1), 400 nm (5 cycles+1), 200 nm (5 cycles+1) and 100 nm (10 cycles+1) filters. The final 100 nm-extruded liposomes were ejected from the syringe in the cell culture hood with proper aseptic techniques to avoid contamination.

The quality of the liposome was checked by diluting 10 μL of the 5-10 mM liposomes into 1000 μL of 0.22 μm filtered 240 mM (NH₄)₂SO₄ solution for DLS size measurement. Zeta potential measurement sample was prepared by diluting 10 μL of the 5-10 mM liposomes into 1000 1000 μL of diH₂O for zeta potential measurements. The targeted liposome size should not be larger than 125 nm for the 100 nm filter with a PDI<0.1. The liposomes were further extruded if they filed to meet the size/PDI requirements.

To create a cross-bilayer pH-gradient that is required for remote liposomal drug loading, the exterior buffer of the liposome was exchanged into 0.22 μm filtered 1× phosphate buffered saline (PBS) of HEPES buffered saline (HBS) at pH 7.4. The later provide a better stability across a wider temperature range but both can be used interchangeably.

The buffer exchange was achieved by using de-salting size-exclusion column PD-10 (Sephadex G-25). The PD10 elution profile of the liposomes was pre-determined using “Stewart Assay” and determined by UV at 485 nm in CHCl₃ (see, e.g., www.liposomes.org/2009/01/stewart-assay.html).

On a typical run, the maximum sample loading of PD10 was 2.5 mL but recommended to be ≤1 mL for better separation. Liposomes were eluted from 2.75 mL-5.75 mL (3 mL combined).

In practice, the PD-10 column was pre-conditioned by eluting with 50 mL PBS/HBS then loaded with liposome samples in acidic buffers, e.g., the (NH₄)₂SO₄. If 1 mL liposomes dispersion was loaded, the elution volume=1 mL, then added 1.75 mL PBS (0.5 mL+1 mL+0.25 mL) to reach a 2.75 mL elution volume. Once 2.75 mL was reached, a 15 mL centrifuge tube was placed under the PD10 column and add 3 mL PBS/HBS and 3 mL eluent was collected which contains the liposomes. The buffer exchanged liposomes now have 240 mM (NH₄)₂SO₄ solution inside and PBS/HBS outside suitable for remote drug loading.

To perform the drug loading, Mavorixafor (MAV or AMD11070) was prepared as stock solution in PBS and added at 20% w/w over the total wt. of the lipids. The MAV/liposome mixed solution was then heated at 65° C. for 1 h then cooled at room temperature for 30 minutes. The separation of encapsulated liposomal MAV (L-MAV) and non-encapsulated MAV (free MAV) was again, through the use of PD10 size exclusion column.

The elution profile of the free MAV is pre-determined by eluting free MAV through the PD10 and measuring the UV absorbance for each elution fraction at λmax 275 nm. In a typical run a 95%+ MAV recovery will be found between 10-20 mL fractions. The free-MAV fractions was collected and combined to quantify the free-MAV by UV at λmax 275 nm. Again, the L-MAV was collected from elution 2.75-5.75 mL (3 mL in total), the free-MAV was collected from 10-20 mL fractions.

Based on the free-MAV quantification, the drug loading of L-MAV was back calculated by subtracting the free MAV from the initial MAV feeding=20% w/w. Typical MAV loading was around 8-15% w/w. Based on the drug loaded L-MAV concentration (mg/mL), L-MAV was concentrated by using 50 mL VIVA-spin tubes at 30 kDa cut-off to a final concentration of 0.1 mg/mL (=0.5 mg/kg per 200 μL injection for a 20 g mouse).

Following the application of a lipid bilayer, we used the weak basic properties of the of CXCR4 antagonists (AMD3100, AMD3465, AMD070) to attempt silicasome remote loading. To optimize the loading efficiency, two type of trapping agents, triethylammonium sucrose octasulfate (TEA8SOS) and ammonium sulfate ((NH₄)₂SO₄) were tested. Briefly, for lipid coating, 40 mg/mL of the purified MSNPs were soaked in trapping agent solution (80 mM TEA8SOS or 300 mM (NH₄)₂SO₄)) and were added to an ˜50% (w/v) lipids mixture (DSPC/Chol/DSPE-PEG2000, in the molar ratio of 3:2:0.15) solution in ethanol. The MSNP:lipid ratio was 1:1.25 (w/w). The suspension was sonicated using probe sonication (Ultrasonic Processor Model VCX130, 40% amplitude) at a 15 s/15 s on/off cycle.

The free trapping agent was removed through size exclusion chromatography using Sepharose CL-4B resin with a HEPES-buffered dextrose solution (5 mM HEPES, 5% dextrose, pH 6.5) for elution. For drug import, trapping agent-loaded particles were mixed with drug and incubated at 65° C. for 0.5 hour. The drug-laden silicasomes were purified by centrifugation to remove the free drug and liposomes; then filtered with a 0.2 μm membrane for sterilization. The drug concentration was determined by either UV spectroscopy or HPLC. Particle hydrodynamic size and zeta potential were measured by a ZETAPALS instrument (Brookhaven Instruments Corporation). The final product was visualized by cryoEM (TF20 FEI Tecnai-G2) to confirm the uniformity and integrity of the coated lipid bilayer. Table 7 shows the encapsulation efficiency is an loading capacities that were achieved with each of the protonating agents for each drug.

TABLE 7
Loading of CXC44 antagonists in silicasomes.

| | Drug | TEA8SOS | | (NH₄)₂SO₄ | |
|---|---|---|---|---|---|
| Drugs | feed ratio | EE % | LC % | EE % | LC % |
| AMD070 | 10% | 95% | 9.5% | | |
| | 20% | 85% | 17% | | |
| | 50% | 70% | 35% | 68% | 34% |
| AMD3100 | 50% | 51% | 25.5% | 27% | 13.5% |
| AMD3465 | 50% | 25% | 12.5% | 34.5% | 17.3% |

EE: encapsulation efficiency.
LC: loading capacity.
Feed ratio: drug:MSNP w/w/.

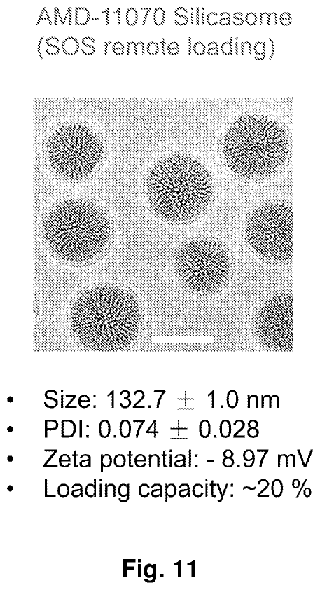

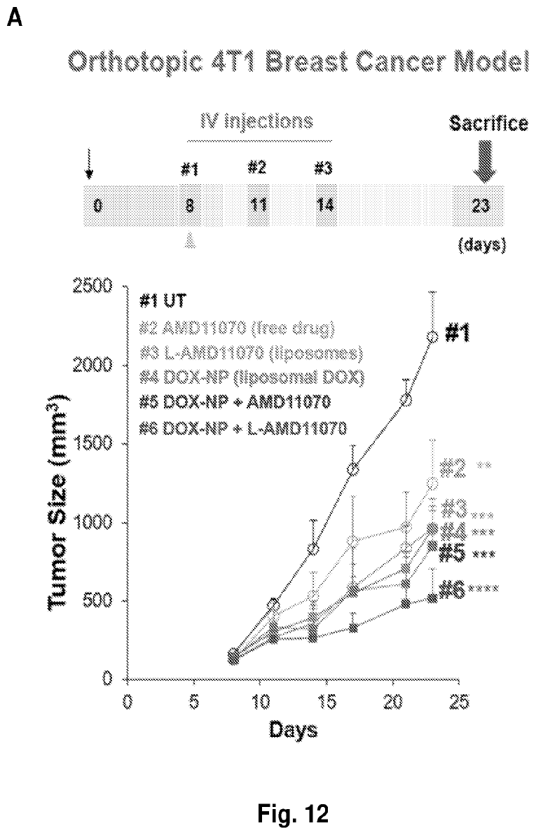

FIG. 11 shows the physicochemical characterization of a silicasome in which sucrose sulfate was used, to accomplish the loading capacity for AMD11070 of 20%. the particle size was 132.7±1.0 nm, PDI 0.074±0.028′ and Zeta potential −8.97 mV.

The first animal study with the AMD11070-liposome was to assess the impact of combination therapy with a liposome delivering Doxorubicin in orthotopic 4T1 and EMT6 tumor models (FIGS. 12-16). Following same-day IV administration of both carriers and the dosing schedule shown in FIG. 12, panel A, tumor shrinkage could be obtained in 4T1 tumors using free AMD11070 alone, liposomal L-AMD11070, DOX-NP®, DOX-NP® plus free AMD11070, and DOX-NP® plus L-AMD11070 (Figure s 19 and 20).

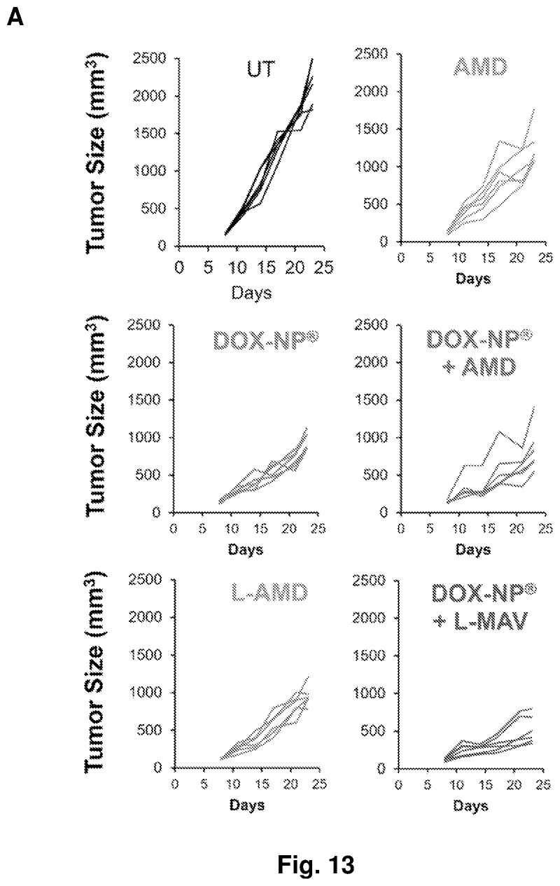

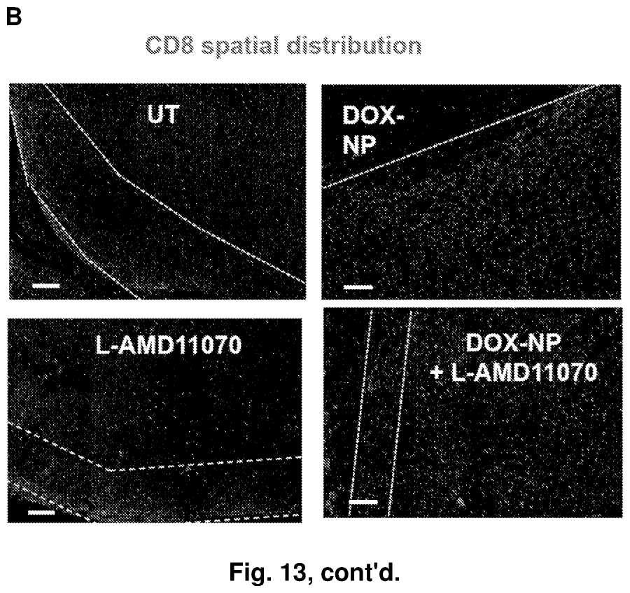

Orthotopic 4T1 tumors were established as described in FIGS. 2-4. These animals develop a high rate of lung metastasis. Liposomal Doxorubicin (DOX-NP) induces significant 4T1 shrinkage (bottom left panel), with evidence of an immunogenic response as shown in above, in FIGS. 2-4. Free AMD11070 alone also leads to tumor shrinkage, which was significantly enhanced when combined with DOX-NP. In addition, combination therapy with DOX-NP plus liposomal AMD11070 provided additional tumor size reduction, in addition to accomplishing the highest CTL recruitment to the tumor core for CTLs excluded in the margin (FIG. 13). FIG. 13 also demonstrates the individual tumor growth curves (spaghetti plots) for the tumors depicted in FIG. 12. The IVIS imaging of explanted animal lungs in FIG. 12 also demonstrate significant metastasis reduction under all conditions where AMD11070 was used.

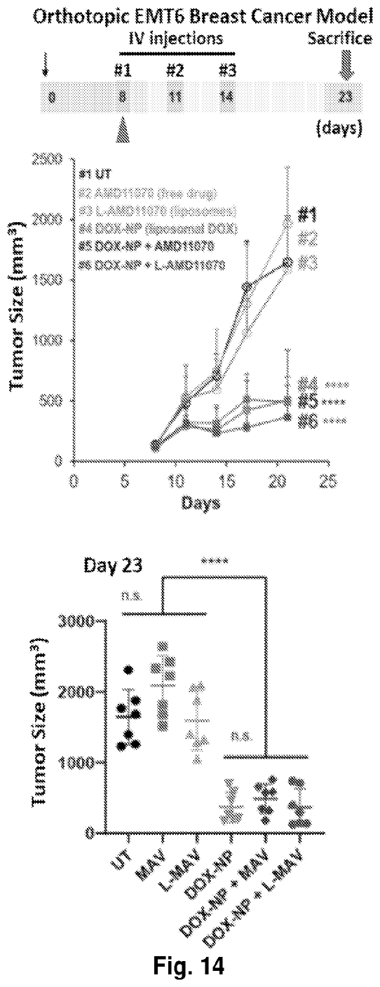

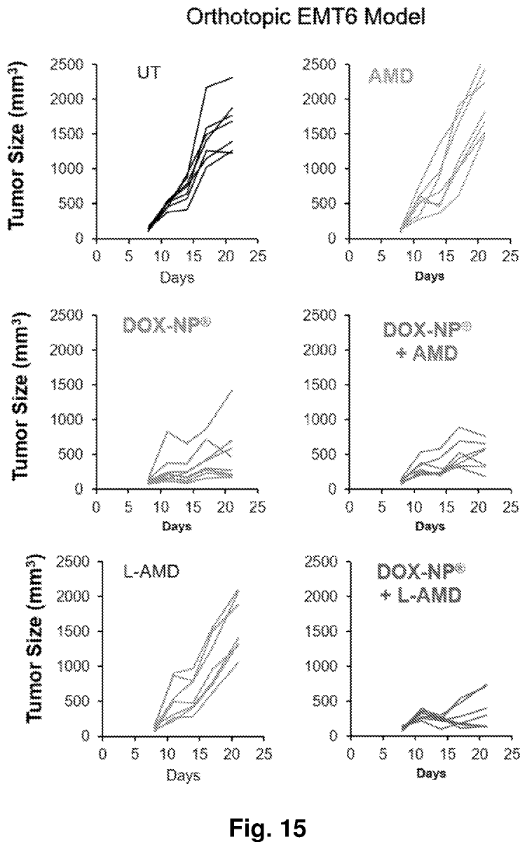

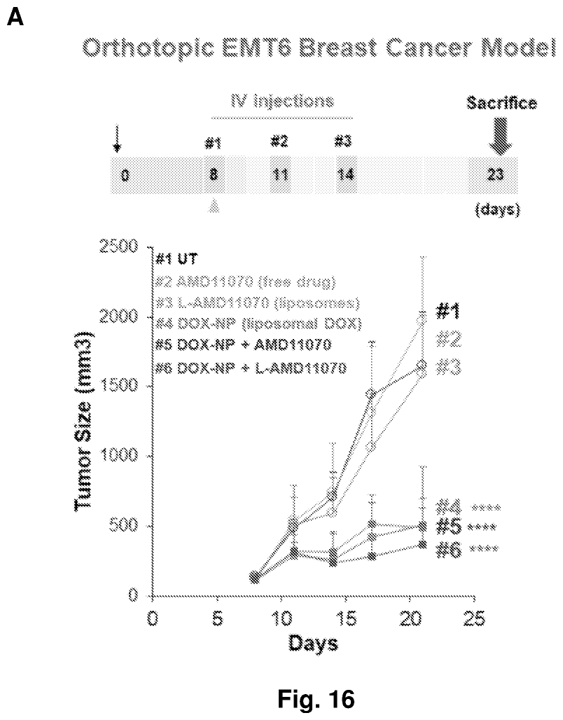

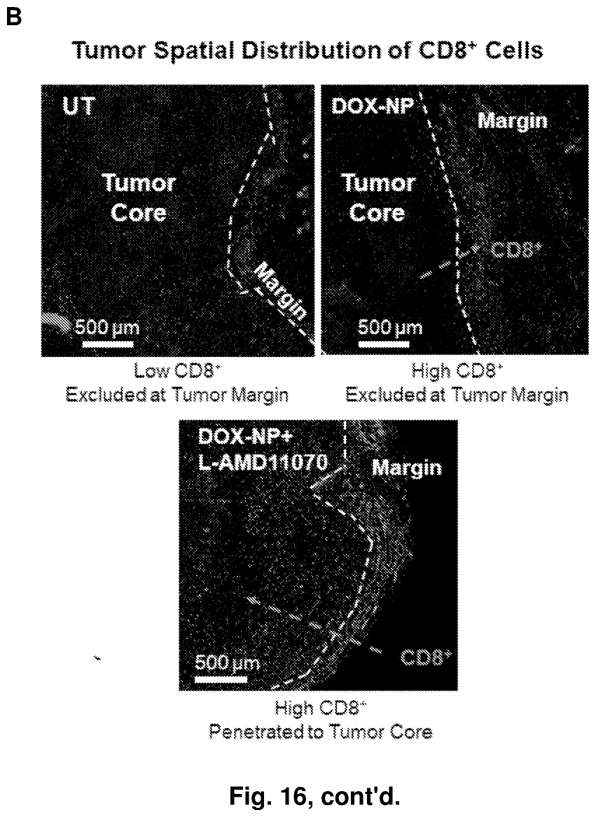

The latter treatment combination resulted in the most significant tumor reduction, with evidence of improved cytotoxic killing (FIG. 12). While both free and encapsulated AMD11070 failed to show a significant effect on CD8+ spatial distribution during mIHC analysis (FIG. 13, panel B), both treatments had a significant impact on lung metastases, in keeping with the important role of CXCR4 in this disease setting (FIG. 7). Similar experimentation in EMT6 demonstrated that co-administration of encapsulated Doxorubicin and AMD11070 resulted in significant tumor shrinkage, albeit that there was no statistical difference with respect to DOX-NP® plus free AMD070 (FIG. 14). Nonetheless, encapsulated AMD11070 was effective in allowing more abundant CTL recruitment to the tumor core (FIG. 16). The corresponding growth curves of individual tumors, accumulated as spaghetti plots, as shown in FIG. 15. This was accompanied by increased perforin deposition at the tumor site and increased cytotoxic killing (not shown).

A similar analysis as in FIGS. 12 and 13 was carried out in the orthotopic EMT6 model, which is characterized by extensive CD8⁺ exclusion from the tumor core, even under basal growth conditions (FIG. 6). In this setting, neither free nor L-AMD11070 were able to interfere in tumor growth (FIGS. 14 and 15). However, combination of free or encapsulated AMD11070 with DOX-NP contributed to growth inhibition, where curiously there was no significant difference between free and encapsulated drugs.

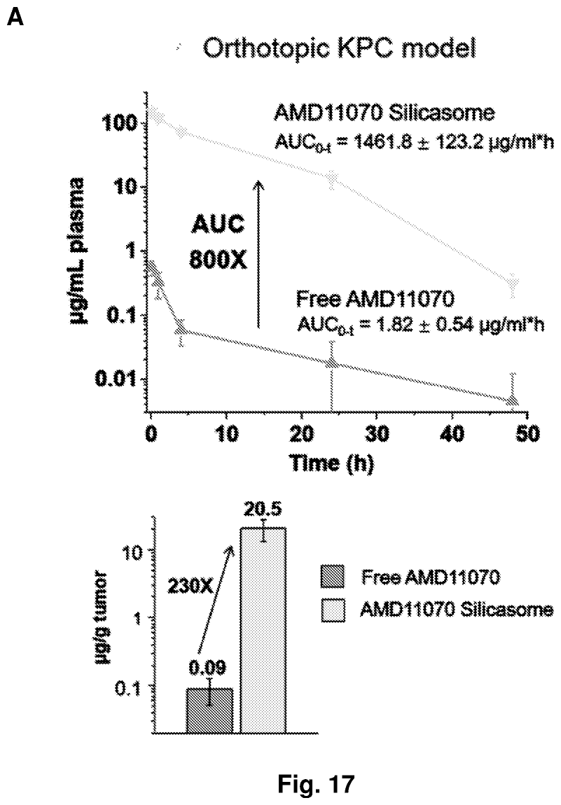

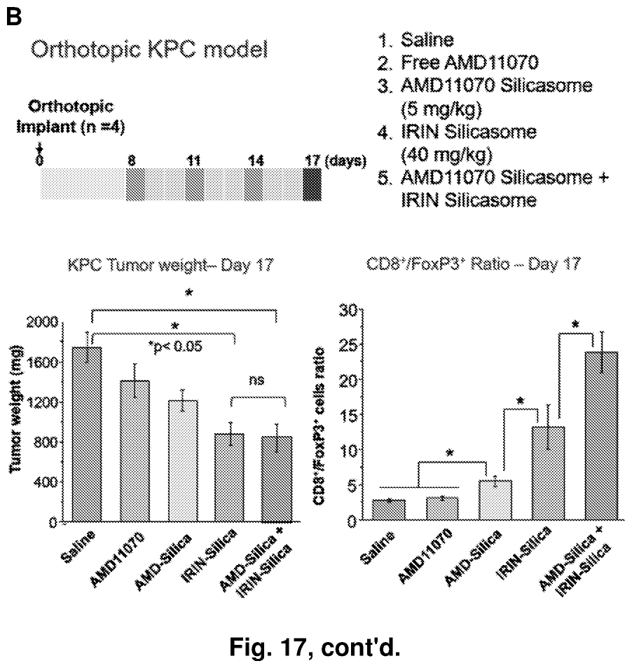

Spatial analysis of the tumor landscape, demonstrated that the increased recruitment of CD8⁺ T-cells during co-administration of DOX-NP, showed a significant shift in cell distribution to the tumor core (FIG. 17). These changes were also accompanied by a reduction in Treg recruitment to the tumor landscape.

The AMD11070-silicasome was used to investigate the impact on drug biodistribution and immunogenic effects in the murine orthotopic KPC model. A PK study was performed, as described in FIG. 17 (panel A). The animals received IV injection of free AMD11070 or AMD11070-silicasome at a drug dose of 5 mg/kg, followed by collection of blood samples at 5 min, 1, 4, 24, and 48 hrs. After separation of the plasma fraction, the drug was extracted in an acidic methanol solution (0.1 mol/L phosphoric acid/methanol, 1:4 v/v). Drug content in the tumor tissue was obtained from KPC tumor bearing animals 24 hr after drug administration. The PK data were analyzed by PKSolver software, using a one-compartment model.

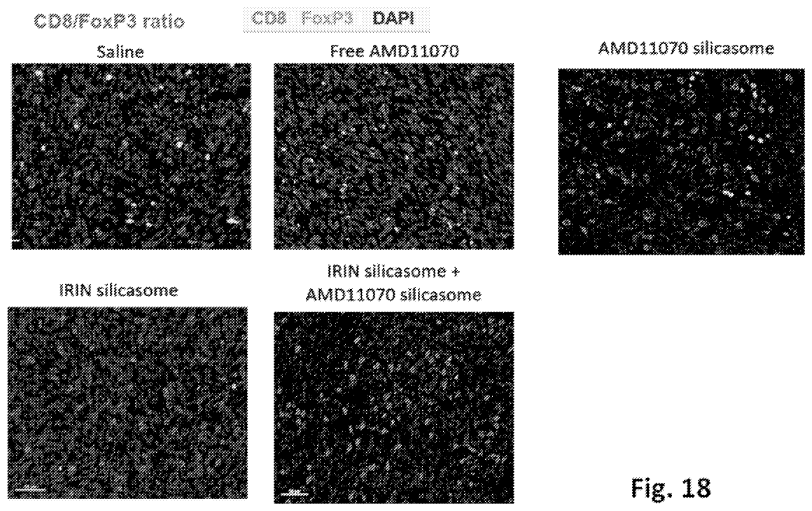

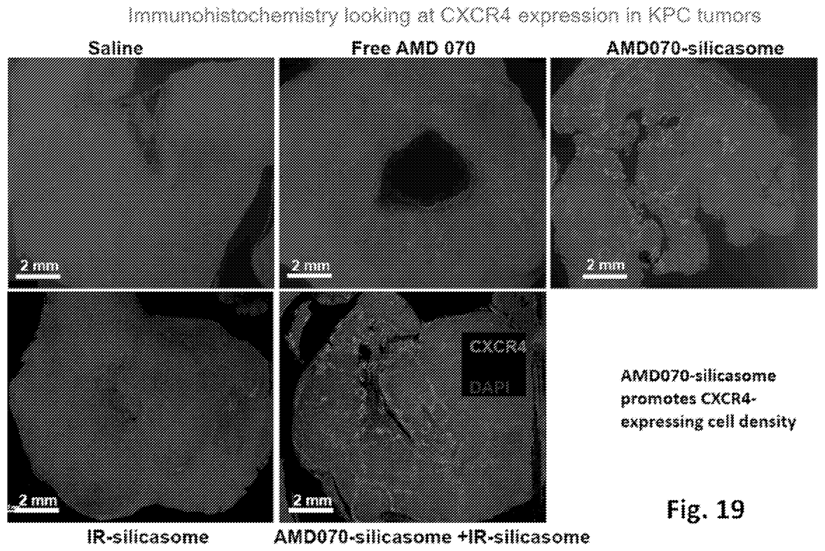

The data demonstrate a significant prolongation of the circulatory half-life by drug encapsulation (FIG. 17, panel A, top), leading to an 800-fold increase in the area under the curve (AUC) and a 230-fold increase in drug delivery at the tumor site (FIG. 17, panel A, bottom). In subsequent experimentation in the KPC orthotopic model, mice were randomly assigned to 5 groups (n=4): saline, free AMD11070, AMD11070 silicasome, IRIN-silicasome, IRIN-silicasome+AMD11070 silicasome (right panel). This was followed by IV administration of a 40 mg/kg Irinotecan dose equivalent or 5 mg/kg AMD070 dose equivalent on 4 occasions, as shown in FIG. 17, panel B. Animals were sacrificed on day 17 before tumor collection and immunophenotyping. While there was no additional tumor shrinkage during co-delivery of the AMD11070 silicasome over the short observation period, there was a significant increase in the CD8/FoxP3 ratio (FIG. 17, panel B and FIG. 18). We also observed increased staining intensity for the CXCR4 receptor in KPC tumors exposed to encapsulated AMD11070 (FIG. 19). Long-term animal survival studies are ongoing.

To complement above results, a number of our previously described dual-delivery carriers can be used to combine CXCR4 antagonists with other therapeutic agents. This includes LB-coated carriers with remote loading of AMD11070 in addition to the encapsulation of IDO-1 or ICI prodrugs in the lipid membrane. CXCR4 antagonists can also be anchored to PEG that is incorporated in a LB of nanocarriers that are also used for loading ICD-inducing drugs or GSK3 inhibitors (Xue et al. (2020) Int. J. Nanomedicine, 15: 5701-5718; Zheng et al. (2019) J. Exp. Clin. Cancer Res. 38: 232). This topological arrangement also allows the conjugated drug to target CXCR4 expressing tumor or metastatic sites.204. An example of customized, nano-enabled combination therapy for TNBC, including use of CXCR4 antagonist appear in Table 8.

TABLE 8
Illustrative nano-enabled combination therapy for TNBC.

| TIME Category | Primary and complementary strategies |
|---|---|
| Ignored or | The primary strategy is to change the “cold” immune status |
| Immune Desert | by encapsulated delivery of ICD inducing chemo: Doxorubicin, |
| Landscape. | Mitoxantrone or Paclitaxel |
| | Depending on the level of success, the consolidation |
| | strategy would be to propagate the immune response by |
| | combination strategies that make use of antibody blockade or |
| | co-formulation of the nanocarrier with prodrugs or SMI that |
| | interfere in PD-1/PD-L1, GSK3, and A2AR and B7-H4* |
| | checkpoint pathways |
| | Key supplementary treatment to augment the outcome |
| | should address stromal fibrosis and presence of immune |
| | suppressive stromal cell types, using carriers that deliver |
| | AMD11070, with possible assistance by losartan or a CSF1R |
| | inhibitor, e.g., pexidartinib**, which interferes in M2 |
| | macrophage activity |
| | Additional consideration could be the inclusion WNT |
| | pathway blockers, e.g. WNT794 |
| Excluded or | The primary strategy is to change the “cold” immune status |
| Stroma- | by ICD therapy as described above |
| restricted | Consolidation strategy as described above, except that B7- |
| Landscape | H4 may not be upregulated in this setting |
| | Key supplementary treatment could target the highly |
| | expressed CXCL12/CXCR4 axis, as well as the stromal |
| | therapies listed above. |
| | Additional considerations could include inhibitors of |
| | VEGF receptor kinases |
| Inflamed | The primary strategy is single or combination ICI therapy, |
| or Fully | but should consider metronomic chemotherapy to maintain the |
| inflamed | activated immune status, |
| Landscape | The consolidation strategy, based on the role of the IFN-γ |
| | mediated JAK-STAT axis feedback loop, is to provide co- |
| | formulated drugs interfering in the PD-1/PD-L1, A2AR and |
| | IDO-1 pathways, possibly from the beginning. |
| | Key supplementary treatment includes the use of type I |
| | IFN stimuli to boost and maintain antigen presentation, as well |
| | as use of a CSF1R inhibitor to target M2 macrophages that are |
| | often also overexpressed in the TME. |
| | Additional consideration should also be given to |
| | intervening in the IFN-γ mediated JAK-STAT axis |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A drug delivery vehicle, said drug delivery vehicle comprising:
a silicasome comprising a mesoporous silica nanoparticle coated with a lipid bilayer and further comprising a CXCR4 antagonist; or
a liposome comprising a lipid bilayer comprising where said liposome further comprises a CXCR4 antagonist.

2. The drug delivery vehicle of claim 1, wherein said drug delivery vehicle comprises a silicasome comprising a mesoporous silica nanoparticle coated with a lipid bilayer and further comprising a CXCR4 antagonist.

3. The drug delivery vehicle of claim 1, wherein said drug delivery vehicle comprises a liposome comprising a lipid bilayer comprising where said liposome further comprises a CXCR4 antagonist.

4. The drug delivery vehicle according to any one of claims 1-3, wherein said CXCR4 antagonist comprises one or more CXCR4 antagonists selected from the group consisting of AMD3100, AMD3465, and AMD070.

5. The drug delivery vehicle according to any one of claims 1-4, wherein said CXCR4 antagonist is disposed within said silicasome or said liposome.

6. The drug delivery vehicle of claim 5, wherein the CXCR4 antagonist is remote loaded into said silicasome or said liposome using a protonating agent.

7. The drug delivery vehicle of claim 6, wherein said protonating agent before reaction with the CXCR4 antagonist is selected from the group consisting of triethylammonium sucrose octasulfate (TEA₈SOS), (NH₄)₂SO₄, an ammonium salt, a trimethylammonium salt, and a triethylammonium salt.

8. The drug delivery vehicle of claim 7, wherein said protonating agent is triethylammonium sucrose octasulfate (TEA₈SOS).

9. The drug delivery vehicle according to any one of claims 1-8, wherein said CXCR4 antagonist is conjugated to a component of the lipid bilayer comprising said silicasome or said liposome.

10. The drug delivery vehicle of claim 9, wherein said CXCR4 antagonist is conjugated to a component of the lipid bilayer selected from the group consisting of a phospholipid, cholesterol, a cholesterol derivative, and a pegylated lipid.

11. The drug delivery vehicle according to any one of claims 1-10, wherein said lipid bilayer comprises a phospholipid and/or a phospholipid prodrug.

12. The drug delivery vehicle of claim 11, wherein said lipid bilayer comprises a phospholipid, and cholesterol (CHOL) and/or a cholesterol derivative.

13. The drug delivery vehicle according to any one of claims 11-12, wherein said phospholipid comprises a saturated fatty acid with a C14-C20 carbon chain, and/or an unsaturated fatty acid with a C14-C20 carbon chain, and/or a natural lipid comprising a mixture of fatty acids with C12-C20 carbon chains.

14. The drug delivery vehicle of claim 13, wherein said phospholipid comprises a saturated fatty acid selected from the group consisting of phosphatidylcholine (DPPC), dimyristoylphosphatidylcholine (DMPC), distearoylphosphatidylcholine (DSPC), diactylphosphatidylcholine (DAPC), and 1,2-distearoyl-sn-glycero-3-phosphoethanolamine (DSPE).

15. The drug delivery vehicle of claim 13, wherein said phospholipid comprises an unsaturated fatty acid selected from the group consisting of 1,2-dimyristoleoyl-sn-glycero-3-phosphocholine, 1,2-dipalmitoleoyl-sn-glycero-3-phosphocholine, 1,2-dioleoyl-sn-glycero-3-phosphocholine (DOPC), and 1,2-dieicosenoyl-sn-glycero-3-phosphocholine.

16. The drug delivery vehicle according to any one of claims 11-15, wherein said lipid bilayer comprises an mPEG phospholipid with a phospholipid C14-C18 carbon chain, and a PEG molecular weight ranging from about 350 Da to 5000 Da.

17. The drug delivery vehicle of claim 16, wherein said lipid bilayer comprises 1,2-distearoyl-sn-glycero-3-phosphoethanolamine-PEG (DSPE-PEG).

18. The drug delivery vehicle of claim 17, wherein said lipid bilayer comprises DPSE-PEG_{2K}.

19. The drug delivery vehicle according to any one of claims 11-18, wherein said lipid bilayer comprises a cholesterol derivative.

20. The drug delivery vehicle of claim 19, wherein said lipid bilayer comprises a cholesterol derivative selected from the group consisting of cholesterol hemisuccinate (CHEMS), lysine-based cholesterol (CHLYS), and PEGylated cholesterol (Chol-PEG).

21. The drug delivery vehicle of claim 20, wherein said lipid bilayer comprises cholesterol hemisuccinate (CHEMS).

22. The drug delivery vehicle of claim 14, wherein said lipid bilayer comprises DSPC and cholesterol (Chol).

23. The drug delivery vehicle of claim 22, wherein said lipid bilayer comprises DSPC, cholesterol (Chol), and a pegylated lipid.

24. The drug delivery vehicle of claim 23, wherein the molar ratio of DSPC:Chol:Pegylated lipid is 3:2:0.15.

25. The drug delivery vehicle according to any one of claims 23-24, wherein said lipid bilayer comprises DSPC, Chol, and DSPE-PEG.

26. The drug delivery vehicle of claim 25, wherein said lipid bilayer comprises DSPC, Chol, and DSPE-PEG2000.

27. The drug delivery vehicle according to any one of claims 1-26, wherein said drug delivery vehicle is conjugated to a moiety selected from the group consisting of a targeting moiety, a fusogenic peptide, and a transport peptide.

28. The drug delivery vehicle according to any one of claims 1-27, wherein said drug delivery vehicle contains a second drug or said second drug is conjugated to a component of the lipid bilayer comprising said silicasome or said liposome.

29. The drug delivery vehicle of claim 28, wherein said drug delivery vehicle contains said second drug.

30. The drug delivery vehicle of claim 28, wherein said second drug is conjugated to a component of the lipid bilayer comprising said silicasome or said liposome.

31. The drug delivery vehicle of claim 30, wherein said second drug is conjugated to a component of the lipid bilayer selected from the group consisting of a phospholipid, cholesterol, a cholesterol derivative, and a pegylated lipid.

32. The drug delivery vehicle of claim 31, wherein second drug is conjugated directly to said component of the lipid bilayer.

33. The drug delivery vehicle of claim 31, wherein said second drug is conjugated to a phospholipid.

34. The drug delivery vehicle of claim 31, wherein said second drug is conjugated to cholesterol.

35. The drug delivery vehicle of claim 31, wherein said second drug is conjugated to a cholesterol derivative.

36. The drug delivery vehicle of claim 31, wherein said second drug is conjugated to a pegylated lipid.

37. The drug delivery vehicle of claim 36, wherein said second drug is conjugated to DSPE-PEG.

38. The drug delivery vehicle according to any one of claims 28-37, wherein said second drug comprises one or more drugs selected from the group consisting of an IDO inhibitor, an immunogenic cell death (ICD)-inducing drug.

39. The drug delivery vehicle of claim 38, wherein said second drug comprises one or more IDO inhibitor(s).

40. The drug delivery vehicle of claim 39, wherein said IDO-1 inhibitor comprises an agent selected from the group consisting of D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-β-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide, N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, and 4-phenylimidazole.

41. The drug delivery vehicle according to any one of claims 38-40, wherein said second drug comprises one or more ICD inducer(s).

42. The drug delivery vehicle of claim 41, wherein said ICD inducer comprises a chemotherapeutic agent selected from the group consisting of doxorubicin, oxaliplatin, anthracenedione, bleomycin, bortezomib, cisplatin, daunorubicin, docetaxel, epirubicin, idarubicin, mitoxanthrone, paclitaxel, R2016, and cyclophosphamide.

43. The drug delivery vehicle according to any one of claims 38-42, wherein said second drug comprises an immune checkpoint inhibitor (ICI).

44. The drug delivery vehicle of claim 43, wherein said checkpoint inhibitor comprises one or more checkpoint inhibitors selected from the group consisting of a PD-L1 inhibitor, a PD-1 inhibitor, a CTLA-4 inhibitor, a PD-L2inhibitor, a PD-L3inhibitor, a PD-L4inhibitor, a LAG3inhibitor, a B7-H3inhibitor, a B7-H4inhibitor, a KIR, and a TIM3 inhibitor.

45. A pharmaceutical formulation, said formulation comprising:
a drug delivery vehicle according to any one of claims 1-44; and
a pharmaceutically acceptable carrier.

46. A method of treating a cancer, said method comprising:
administering to a subject in need thereof an effective amount of a drug delivery vehicle according to any one of claims 1-44 or a pharmaceutical formulation of claim 45.

47. The method of claim 46, wherein said administering to a subject in need thereof an effective amount of a drug delivery vehicle comprises a primary therapy in a chemotherapeutic regimen.

48. The method of claim 46, wherein said administering to a subject in need thereof an effective amount of a drug delivery vehicle comprises an adjunct therapy in a treatment regime that additionally comprises chemotherapy using another chemotherapeutic agent, and/or surgical resection of a tumor mass, and/or radiotherapy.

49. The method of claim 46, wherein said drug delivery vehicle and/or said pharmaceutical formulation is a component in a multi-drug chemotherapeutic regimen.

50. The method according to any one of claims 46-49, wherein said cancer is a cancer selected from the group consisting of breast cancer, lung cancer, melanoma, pancreas cancer, liver cancer, acute lymphoblastic leukemia (ALL), acute myeloid leukemia (AML), adrenocortical carcinoma, AIDS-related cancers (e.g., Kaposi sarcoma, lymphoma), anal cancer, appendix cancer, astrocytomas, atypical teratoid/rhabdoid tumor, bile duct cancer, extrahepatic cancer, bladder cancer, bone cancer (e.g., Ewing sarcoma, osteosarcoma, malignant fibrous histiocytoma), brain stem glioma, brain tumors (e.g., astrocytomas, brain and spinal cord tumors, brain stem glioma, central nervous system atypical teratoid/rhabdoid tumor, central nervous system embryonal tumors, central nervous system germ cell tumors, craniopharyngioma, ependymoma, burkitt lymphoma, carcinoid tumors (e.g., childhood, gastrointestinal), cardiac tumors, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, duct cancers e.g. (bile, extrahepatic), ductal carcinoma in situ (DCIS), embryonal tumors, endometrial cancer, ependymoma, esophageal cancer, esthesioneuroblastoma, extracranial germ cell tumor, extragonadal germ cell tumor, extrahepatic bile duct cancer, eye cancer (e.g., intraocular melanoma, retinoblastoma), fibrous histiocytoma of bone, malignant, and osteosarcoma, gallbladder cancer, gastric (stomach) cancer, gastrointestinal carcinoid tumor, gastrointestinal stromal tumors (GIST), germ cell tumors (e.g., ovarian cancer, testicular cancer, extracranial cancers, extragonadal cancers, central nervous system), gestational trophoblastic tumor, brain stem cancer, hairy cell leukemia, head and neck cancer, heart cancer, hepatocellular (liver) cancer, histiocytosis, langerhans cell cancer, Hodgkin lymphoma, hypopharyngeal cancer, intraocular melanoma, islet cell tumors, pancreatic neuroendocrine tumors, kaposi sarcoma, kidney cancer (e.g., renal cell, Wilm's tumor, and other kidney tumors), langerhans cell histiocytosis, laryngeal cancer, leukemia, acute lymphoblastic (ALL), acute myeloid (AML), chronic lymphocytic (CLL), chronic myelogenous (CML), hairy cell, lip and oral cavity cancer, liver cancer (primary), lobular carcinoma in situ (LCIS), lung cancer (e.g., childhood, non-small cell, small cell), lymphoma (e.g., AIDS-related, Burkitt (e.g., non-Hodgkin lymphoma), cutaneous T-Cell (e.g., mycosis fungoides, Sézary syndrome), Hodgkin, non-Hodgkin, primary central nervous system (CNS)), macroglobulinemia, Waldenström, male breast cancer, malignant fibrous histiocytoma of bone and osteosarcoma, melanoma (e.g., childhood, intraocular (eye)), merkel cell carcinoma, mesothelioma, metastatic squamous neck cancer, midline tract carcinoma, mouth cancer, multiple endocrine neoplasia syndromes, multiple myeloma/plasma cell neoplasm, mycosis fungoides, myelodysplastic syndromes, Myelogenous Leukemia, Chronic (CML), multiple myeloma, nasal cavity and paranasal sinus cancer, nasopharyngeal cancer, neuroblastoma, oral cavity cancer, lip and oropharyngeal cancer, osteosarcoma, ovarian cancer, pancreatic cancer, pancreatic neuroendocrine tumors (islet cell tumors), papillomatosis, paraganglioma, paranasal sinus and nasal cavity cancer, parathyroid cancer, penile cancer, pharyngeal cancer, pheochromocytoma, pituitary tumor, plasma cell neoplasm, pleuropulmonary blastoma, primary central nervous system (CNS) lymphoma, prostate cancer, rectal cancer, renal cell (kidney) cancer, renal pelvis and ureter, transitional cell cancer, rhabdomyosarcoma, salivary gland cancer, sarcoma (e.g., Ewing, Kaposi, osteosarcoma, rhadomyosarcoma, soft tissue, uterine), Sézary syndrome, skin cancer (e.g., melanoma, merkel cell carcinoma, basal cell carcinoma, nonmelanoma), small intestine cancer, squamous cell carcinoma, squamous neck cancer with occult primary, stomach (gastric) cancer, testicular cancer, throat cancer, thymoma and thymic carcinoma, thyroid cancer, trophoblastic tumor, ureter and renal pelvis cancer, urethral cancer, uterine cancer, endometrial cancer, uterine sarcoma, vaginal cancer, vulvar cancer, Waldenström macroglobulinemia, and Wilm's tumor.

51. The method according to any one of claims 46-50, wherein said drug delivery vehicle is coadminstered with a second drug.

52. The method of claim 51, wherein said second drug comprises one or more drugs selected from the group consisting of an IDO-1 inhibitor, an immunogenic cell death (ICD)-inducing drug.

53. The method of claim 52, wherein said second drug comprises one or more IDO inhibitor(s).

54. The method of claim 53, wherein said IDO-1 inhibitor comprises an agent selected from the group consisting of D-1-methyl-tryptophan (indoximod, D-1MT), L-1-methyl-tryptophan (L-1MT), a mixture of D-1MT and L-1MT, 1-methyl-L-tryptophan (L-1MT), methylthiohydantoin-dl-tryptophan (MTH-Trp, Necrostatin), β-carbolines (e.g., 3-butyl-β-carboline), Naphthoquinone-based (e.g., annulin-B), S-allyl-brassinin, S-benzyl-brassinin, N-[2-(Indol-3-yl)ethyl]-S-methyl-dithiocarbamate, N-[2-(benzo[b]thiophen-3-yl)ethyl]-S-methyl-dithiocarbamate, N-[3-(Indol-3-yl)propyl]-S-methyl-dithiocarbamate, S-hexyl-brassinin, N-[2-(indol-3-yl)ethyl]-S-benzyl-dithiocarbamate, N-[2-(indol-3-yl)ethyl]-S[(naphth-2-yl)methyl]-dithiocarbamate, N-[2-(indol-3-yl)ethyl]-S-[(pyrid-3-yl)methyl]-dithiocarbamate, N-[2-(indol-3-yl)ethyl]-S-[(pyrid-4-yl)methyl]-dithiocarbamate, 5-bromo-brassinin, Phenylimidazole-based IDO inhibitors (e.g., 4-phenylimidazole), Exiguamine A, imidodicarbonimidic diamide, N-methyl-N′-9-phenanthrenyl-monohydrochloride (NSC401366), INCB024360 (epacadostat), 1-cyclohexyl-2-(5H-imidazo[5,1-a]isoindol-5-yl)ethanol (GDC-0919), IDO1-derived peptide, NLG919, Ebselen, Pyridoxal Isonicotinoyl Hydrazone, Norharmane, CAY10581, 2-Benzyl-2-thiopseudourea hydrochloride, and 4-phenylimidazole.

55. The method according to any one of claims 52-54, wherein said second drug comprises one or more ICD inducer(s).

56. The method of claim 55, wherein said ICD inducer comprises a chemotherapeutic agent selected from the group consisting of doxorubicin, oxaliplatin, anthracenedione, bleomycin, bortezomib, cisplatin, daunorubicin, docetaxel, epirubicin, idarubicin, mitoxanthrone, paclitaxel, R2016, and cyclophosphamide.

57. The method according to any one of claims 52-56, wherein said second drug comprises an immune checkpoint inhibitor (ICI).

58. The method of claim 57, wherein said checkpoint inhibitor comprises one or more checkpoint inhibitors selected from the group consisting of a PD-L1 inhibitor, a PD-1 inhibitor, a CTLA-4 inhibitor, a PD-L2inhibitor, a PD-L3inhibitor, a PD-L4inhibitor, a LAG3inhibitor, a B7-H3inhibitor, a B7-H4inhibitor, a KIR, and a TIM3 inhibitor.
